(19) United States
(12) Patent Application Publication
(10) Pub. No.: US 2024/0019353 A1
(43) Pub. Date: Jan. 18, 2024

(54) TRANSCRIPTOMICS WITH ELECTROPHYSIOLOGICAL RECORDING

(71) Applicants: The Broad Institute, Inc., Cambridge, MA (US); Massachusetts Institute of Technology, Cambridge, MA (US); President and Fellows of Harvard College, Cambridge, MA (US)

(72) Inventors: Xiao Wang, Cambridge, MA (US); Jia Liu, Cambridge, MA (US)

(73) Assignees: The Broad Institute, Inc., Cambridge, MA (US); Massachusetts Institute of Technology, Cambridge, MA (US); President and Fellows of Harvard College, Cambridge, MA (US)

(21) Appl. No.: 18/033,754

(22) PCT Filed: Oct. 27, 2021
(86) PCT No.: PCT/US2021/056821

§ 371 Date: Apr. 25, 2023

Related U.S. Application Data
(60) Provisional application No. 63/178,864, filed on Apr. 23, 2021.
(60) Provisional application No. 63/106,292, filed on Oct. 27, 2020.

(51) Int. Cl. G01N 15/10; G01N 27/327; C12Q 1/6869

(52) U.S. Cl. CPC G01N 15/1031; G01N 27/3278; C12Q 1/6869; G01N 2015/1006

(57) ABSTRACT

Disclosed herein are methods and systems for correlating continuous physiological processes (e.g., electrophysiological activity) and biomolecular processes (e.g., gene expression) in cells within a tissue. Also disclosed herein are methods for preparing a tissue for continuous electrophysiological recording. Further disclosed herein are systems comprising nanoelectronic devices within cells in a tissue, wherein each nanoelectronic device comprises a unique electronic barcode. The methods and systems described herein comprise any tissue with electrical activity (e.g., brain tissue, heart tissue, nervous system tissue, muscle tissue, pancreas tissue, or gastrointestinal tract tissue). Additionally disclosed herein are methods for disease modeling, methods for discovering a target for treating a disease, and methods for drug screening.

### RELATED APPLICATIONS

This application claims priority under 35 U.S.C. § 119(e) to U.S. Provisional Application Ser. No. 63/178,864, filed Apr. 23, 2021, and U.S. Provisional Application Ser. No. 63/106,292, filed Oct. 27, 2020, each of which is incorporated herein by reference.

### GOVERNMENT SUPPORT

This invention was made with government support under grant number 1RF1MH123948-01 awarded by the National Institutes of Health and grant number ECCS-2038603 awarded by the National Science Foundation. The government has certain rights in the invention.

### BACKGROUND OF THE INVENTION

Fully understanding the function of tissues and organs (e.g., the brain, the heart, etc.) relies on the interrogation of the spatiotemporally orchestrated communication of different types of cells distributed across the three-dimensional (3D) volume of a tissue over the time course related to their development, function, and maturation (Alivisatos, A. P. et al. Science. 2013, 339, 1284-1285; Wang, X. et al. Science. 2018, 361, 1-9). Bioelectronics has been advanced to allow probing the multimodal (e.g., electrical, mechanical, etc.) physiological activities of a large number of cells, both in vitro and in vivo, defining cellular functional states at millisecond and single-cell spatiotemporal resolution in a long-term, stable manner (Liu, J. et al. Proc. Natl. Acad. Sci. USA 2013, 110, 6694-6699; Liu, J. et al. Nature Nanotechnology. 2015, 10, 629; Li, Q. et al. Nano Lett. 2019, 19, 5781-5789; Fu, T. M. et al. Nature Methods. 2016, 13, 875). Single-cell RNA sequencing (scRNAseq) has demonstrated that cell type and transcriptional and transcriptomic states can be discovered and defined at single-cell resolution. The ability to combine state-of-the-art bioelectronics with scRNAseq will allow for the integration of continuous multimodal physiological interrogations with cell type and state mapping at single-cell resolution (Keller, P. J. & Ahrens, M. B. Neuron, 2015, 462-483; Rosenberg, A. B. et al. Science. 2018, 360, 176-182; Toga, A. W. et al. Nat Rev Neurosci. 2006, 7, 952-966).

Currently, the Patch-seq approach has been developed to probe cellular transient electrical states (i.e., electrophysiology) and transcriptional states from the same cell (Kodandaramaiah, S. B. et al. Nature Methods. 2012, 9, 585-587; Cadwell, C. R. et al. Nat Biotechnol. 2016, 34, 199-203; Fuzik, J. et al. Nat Biotechnol. 2016, 34, 175-183). However, Patch-seq has limited throughput, accessibility, and longevity in 3D tissue. Optical mapping combined with genetically-encoded fluorescence proteins and scRNAseq can offer high throughput interrogation of cellular functional and transcriptional states, yet cannot integrate the data at single-cell resolution. Furthermore, long-term tracing of single-cell activities across 3D tissue is a challenge for optical imaging. There is a need for methods capable of long-term tracing of single-cell electrophysiological activity and gene expression.

### SUMMARY OF THE INVENTION

“Tissue-like” electronics have recently been developed, in which high-performance nanoelectronic sensing units have been embedded into a tissue-level flexible, mesh-like network, capable of forming seamless integration with 3D tissue networks and tracing the multimodal activity of the same cell over months to years (Liu, J. et al. Proc. Natl. Acad. Sci. USA 2013, 110, 6694-6699; Tian, B. et al. Nat. Mater. 2012, 11, 986-994; Liu, J. et al. Nature Nanotechnology. 2015, 10, 629; Li, Q. et al. Nano Lett. 2019, 19, 5781-5789; Fu, T. M. et al. Nature Methods. 2016, 13, 875). Thousands to millions of nanoelectronic sensors may be embedded within a tissue composed of millions of cells. Moreover, an in situ scRNAseq technique (STARmap) has been developed, capable of spatially mapping 200 to 1000 gene markers in an intact tissue network at single-cell resolution through confocal fluorescence imaging (Wang, X. et al. Science. 2018, 361, 1-9).

Disclosed herein is a combination of these techniques to establish a scalable method capable of profiling multimodal physiological activity (e.g., electrophysiological activity) and biomolecular processes (e.g., gene expression) from the same cells in an intact tissue network through (i) unique electronic barcode techniques that label individual electronic sensors for optical imaging, including, but not limited to, lithographically defined shape, number, fluorescence-labeling, and field-effect transistor photocurrent (Liu, J. et al. Proc. Natl. Acad. Sci. USA 2013, 110, 6694-6699); and (ii) hydrogel-tissue-electronic chemistry that limits the local shifting of sensor position to the recording cells. This method displays high throughput, scalability, and longevity. Thousands to millions of nanoelectronic sensors can be embedded into a tissue composed of millions of cells. This powerful multimodal mapping is used to interrogate cellular functional and transcriptional states from tissues. This multimodal data integration will further open up the opportunity to build predictive models using continuous multifunctional recording for real-time prediction of gene expression and decision making to control the underlying transcriptional process.

The present disclosure provides methods for correlating a continuous physiological process (e.g., electrophysiological activity) and a biomolecular process (e.g., gene expression) in a cell in a tissue. Further disclosed herein are systems comprising nanoelectronic devices within cells in a tissue, wherein each nanoelectronic device comprises a unique electronic barcode. Also disclosed herein are methods for preparing a tissue for continuous electrophysiological recording. Additionally, any of the methods or systems described herein can be used for disease modeling, for drug screening, and/or for the discovery of new targets for the treatment of diseases (e.g., neurological diseases, cardiovascular diseases, diseases associated with organ development, etc.). These methods allow for long-term tracing and correlation of single-cell electrophysiological activity and gene expression, allowing the impacts of electrophysiology and gene expression on tissue development to be investigated in ways not previously possible.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1E Tissue-electronics integration: concept and design. FIG. 1A shows schematics that illustrate the stepwise integration of stretchable mesh nanoelectronics into a 3D developing tissue. FIG. 1B shows an exploded view of the stretchable mesh nanoelectronics design consisting of (from top to bottom) a 400 nm-thick top SU-8 encapsulation layer, a 50 nm-thick platinum (Pt) electrode layer coated with poly(3,4-ethylenedioxythiophene) (PEDOT), a 40 nm-thick gold (Au) interconnects layer, and a 400 nm-thick bottom SU-8 encapsulation layer. The inset shows the serpentine layout of the mesh. FIG. 1C is an optical image of stretchable mesh nanoelectronics before being released from the fabrication substrate. The inset shows the zoomed-in view of a single Pt electrode coated with PEDOT. FIGS. 1D, 1E are optical images of released stretchable mesh nanoelectronics in water, folded (FIG. 1D) and stretched (FIG. 1E) by tweezers.

FIGS. 2A-2G Chronic, multiplex, tissue-wide electrophysiological mapping of a cardiac organoid during organogenesis. FIG. 2A is a schematic that shows the setup that connects the electronics-integrated cardiac organoids (referred to as “cyborg cardiac organoids”) to external recording equipment for multiplexing electrophysiology. FIG. 2B shows optical images at 0, 24, and 48 h of the 3D organization of cardiac cyborg organoids in the culture chamber for electrophysiological recording. FIG. 2C shows 14-Channel voltage traces recorded from the cardiac cyborg organoid at day 35 of differentiation. FIG. 2D shows a zoomed-in view of the shadedbox-highlight in FIG. 2C showing a single-spiked field potential recording. FIG. 2E shows a zoom-in view of the dashed box from FIG. 2C on three different culturing days (day 26, 31, and 35 of differentiation). FIGS. 2F and 2G show an amplitude of fast peak (FIG. 2F) and field potential duration (FIG. 2G) defined in FIG. 2E as a function of differentiation time. Gray lines show individual channels. Black line shows the averaged results from 14 channels. (Value=mean±s.e.m., n=14)

FIGS. 3A-3J In situ electrode sequencing: concept and design. FIG. 3A provides schematics showing the in situ electrode sequencing design with the nanoelectronics embedded in the 3D tissue for electrophysiological recording before in situ RNA sequencing. Integration of three-dimensional (3D) in situ sequencing and single-cell electrophysiology in a tissue network is shown. The flexible macroporous electronics are embedded across a 3D tissue network for continuous electrical recording at single-cell resolution. After electrical recording, the tissue-electronics hybrid is fixed for in situ sequencing to read out the spatially-resolved gene expression information for each cell. Each electronic sensor is labeled by a fluorescent electronic barcode (E-barcode) defined by lithographic patterning (highlighted in dashed boxes) to spatially register the sensor-to-cell position during imaging, which integrates the electrical recording and gene expression at the single-cell level. The integrated electrical and gene expression mapping is used for multimodal analysis such as joint clustering and cross-modal prediction. FIG. 3B shows the design of the barcoded nanoelectronics. FIGS. 3C and 3E show electrophysiological features and gene expression profile spatially mapped with sensor position in the black dashed box. FIG. 3C provides schematics showing that in situ RNA sequencing of the tissue-nanoelectronics hybrid starts with the probe hybridizing to the mRNA followed by ligation, rolling circle amplification, gel embedding, and sequencing. The custom padlock probe and primer hybridize to the intracellular mRNA of the 3D tissue-electronics hybrid, followed by enzymatic ligation and rolling circle amplification (RCA) to construct the in situ cDNA amplicons. The amplicons are then copolymerized with acrylamide, forming the hydrogel network. A gene-specific identifier (indicated by an arrow in the hybridization panel) in the probe is amplified through this process, which can then be used for sequencing. After in situ sequencing of cDNA amplicons in the tissue-electronics-hydrogel network, the gene-specific identifier is decoded through multiple sequencing cycles. FIG. 3D shows a 3D reconstructed fluorescence image of cyborg cardiac tissue after in situ RNA sequencing. FIG. 3E shows five rounds of sequencing of a gene-specific identifier (black line on the bottom) in a cDNA amplicon. In each round, the reading probe (line on the left) increased one degenerative base N at 5′P as the start position; the fluorophore at the 3′ end of the decoding probe (line on the right with a star-symbol label) is 2-base labeled (shown in FIG. 3C). After imaging, the reading and decoding probe are stripped away with 60% formamide. Representative images of five rounds of sequencing and co-imaging of E-barcode are shown. In each sequencing cycle, the reading probe has one increasing number of degenerative base N, which sets off the starting position for sequencing at the 5′-phosphate; the decoding probes (line on the right with a star-symbol label) are labeled with a fluorophore at the 3′-end according to the 2-base encoding diagram. Both the reading probe and the decoding probe hybridize to the gene-specific identifier, followed by ligation and imaging. After imaging, both reading and decoding probes are stripped away with 60% formamide. X, unknown base; underline, decoded sequence; Ch1 to Ch4, fluorescence channels; E-barcodes labelled with Rhodamine 6G (R6G). FIG. 3F provides a representative photograph of the flexible mesh electronics for cardiac patch integration. Inset: a schematic illustrating the multilayer structure of the flexible mesh electronics is provided. FIG. 3G demonstrates that overlap of fluorescence and bright-field (BF) images shows a pair of binary E-barcodes that label one electrode channel from the box highlighted region in FIG. 3F. FIG. 3H shows impedance and phase from 0.1 to 10 kHz of one representative electrode. FIGS. 3I-3J show averaged electrochemical impedance of electrodes across five representative mesh electronics at 1 kHz and averaged impedance of electrodes at 1 kHz as a function of incubation time in PBS at 37° C. from one representative stretchable electronics (n=64 electrodes for each device, value=mean +/−s.e.m., two-tailed, unpaired, t-test).

FIGS. 4A-4J Spatial mapping between electrophysiology and gene expression profile. FIG. 4A provides schematics showing the experimental and computational pipeline for in situ electrode sequencing. FIG. 4B shows a 3D reconstructed fluorescence image with full view of interwoven nanoelectronics/cell cDNA amplicons after the first round of sequencing. FIG. 4C provides a 3D reconstructed image showing the sensor registering with cells. FIG. 4D shows 3D reconstructed fluorescence images of amplicons across five sequencing rounds and barcode imaging in the sensor neighborhood. FIG. 4E shows 12-Channel voltage traces (out of 64 channels) recorded from the cardiac cyborg organoids on day 21 (left) and day 31 (right) of differentiation. FIG. 4F shows the correspondence matrix between cardiomyocytes defined by electrophysiological features (E1/E2) and gene expression (CM1/CM2). FIG. 4G shows the top 20 enriched genes in cyborg cardiac tissues. FIG. 4H is a uniform manifold approximation and projection (UMAP) plot showing two (2) electrophysiological clusters, E1 and E2, with the corresponding spike waveform. FIG. 4I shows spatial mapping between electrophysiology in (FIG. 4H) and gene expression in (FIG. 4J) using the registered sensor position (FIG. 4C). FIG. 4J is a UMAP plot (left) and heatmap plot (right) show 3 cell clusters and their corresponding gene expression profile.

FIG. 5 shows a schematic for in situ single cell RNA sequencing.

FIG. 6 shows electrophysiological recording data based on cell type.

FIG. 7 shows gene expression in cells based on cell type.

FIGS. 8A-8B show spatial mapping of cell sequencing data at different time points (days 21, 31, and 46).

FIG. 9 shows a schematic for correlating electrophysiology and gene expression in living cells.

FIG. 10 shows a computational pipeline for correlating electrophysiology data with gene expression data based on cell type.

FIG. 11 shows temporal evolution of electrophysiology and gene expression based on cell type.

FIGS. 12A-12J show that in situ electro-seq integrates single-cell transcriptional and electrophysiological states of human cardiac patch. FIG. 12A provides schematics of the experiment: flexible mesh electronics with a 64-channel subcellular size electrode array are embedded with human induced pluripotent stem cell-derived cardiomyocyte (hiPSC-CM) patch for continuous electrical recording (top left). In situ electro-seq records the extracellular action potential (top middle), profiles spatially-resolved RNA expression (bottom row), and integrates multimodal data at the single-cell level (top right). FIG. 12B shows representative 16-channel voltage traces recorded from cardiac patch at Day 46 of differentiation. FIG. 12C shows the representative 16-channel averaged single spike waveform of action potential detected from 1-min recording from FIG. 12B. The inset shows the uniform manifold approximation and projection (UMAP) visualization of the spike waveforms from 64 channels. Black dots highlight the distribution of the averaged spike waveform for each channel. FIG. 12D shows raw fluorescence imaging of in-process in situ electro-seq for 201 genes with the full view of the entire tissue-electronics hybrid. Arrows highlight positions of 64 electrodes. FIG. 12E shows that a zoomed-in view of the raw fluorescent signals illustrates the representative electrode embedded area from the dashed box highlighted regions in FIG. 12D. FIG. 12F shows that the 3D cell segmentation map generated by ClusterMap labels cells. FIG. 12G shows that the UMAP visualization represents major cell types across 32,429 cells in the entire cardiac patch clustered by Leiden clustering. FIG. 12H shows that the 3D cell-type map labels each cell by its cell type as in FIG. 12G. The electrically recorded cell is highlighted in black, contrasted with the nanoelectronic device shown in white. FIG. 12I is a heatmap showing the extracted features from the waveform of averaged spikes and the corresponding 24 top differentially expressed genes expressed in the electrically recorded cells. Normalized electrophysiological feature value (left) and gene expression value (right) are shown. FIG. 12J shows integration of electrophysiological recording with gene expression features for each cell in UMAP visualization by identifying electrode-to-cell positions through imaging of E-barcodes.

FIGS. 13A-13F show that in situ electro-seq enables joint clustering of cell states in 3D human cardiac tissue maturation. FIG. 13A provides overview schematics illustrating the application of in situ electro-seq to 3D hiPSC-CM patches at different stages. hiPSC-CM patches are integrated with flexible mesh electronics and fixed for in situ electro-seq at Day 12, Day 21, Day 46, and Day 64 of differentiation. Integrated heatmap plots electrophysiological features from 162 cells across four (4) samples and their corresponding 20 top differentially expressed gene expression profiles. FIG. 13B shows hiPSC-CM transcriptional states (t-states, TS) defined by gene expression. UMAP visualization of gene expression of electrically recorded CMs that are shaded by differentiation days (i) and t-states defined by Leiden clustering (ii). Comparison of t-states and differentiation days by river plot (iii) and dot plot (iv). Size of dots represents the percentages of cells from given differentiation days that match to the corresponding t-states. Each row sums to 100%. FIGS. 13C-13D show hiPSC-CM electrophysiological states (e-states, ES) and transcriptional/electrophysiological joint states (j-states, JS) defined by electrophysiology (FIG. 13C) and WNN-integrated representations from gene expression and electrophysiology, respectively, are analyzed as in FIG. 13B. FIG. 13E provides distribution plots showing pseudotime distributions of all the electrically recorded CMs that are learned from Monocle3 using gene expression. The pseudotime are normalized to 0-1. Gene expression (i), electrophysiology (ii), and Weighted Nearest Neighbor (WNN)-integrated representations of gene expression and electrophysiology (iii). FIG. 13F shows electrically recorded cells highlighted in the UMAP visualization of gene expression with shading showing joint pseudotime in (FIG. 13E, iii). All 130,162 cells sequenced from samples across four stages are shown as gray embedding. All 112,892 CMs sequenced from samples across four stages are shown with shading showing pseudotime. Insets show representative single-spike waveforms from Day 12, Day 21, Day 46, and Day 64 of differentiation.

FIGS. 14A-14I show that in situ electro-seq enables cross-modal visualization, correlation, prediction, and mapping. FIG. 14A shows that 62 representative electrophysiological features are extracted through down sampling of each spike waveform on Day 12, Day 21, Day 46, and Day 64 of differentiation, respectively. 1.6-second waveforms are sampled to 20 bins. Inset: 0.15-second fast spikes are sampled to 42 bins. FIG. 14B shows a sparse reduced-rank regression (RRR) model to visualize and align t-states and e-states. Components 1 and 2 of the rank-5 model are shown, n=162. The model selects 40 and 12 ion channel-related genes to train the model for visualization. FIG. 14C provides schematics showing physiological functions of genes that are mostly correlated to electrophysiological feature changes during differentiation identified by the RRR model in (FIGS. 14A-14B) and FIGS. 22E-22F. FIG. 14D provides schematics showing the structure of a coupled autoencoder for electrophysiology-to-transcripts (E-to-T) prediction. Encoders (ε) compress input data (X) from E and T modality into low-dimensional representations (Z), whereas decoders (D) reconstruct data (x̅) from representations. FIG. 14E is a heatmap showing single-cell electrophysiological features continuously recorded from the same cardiac patch over the time course of maturation. FIG. 14F is a heatmap showing the single-cell gene expression profiles predicted by coupled autoencoder (FIG. 14D) using data in FIG. 14E. The 13 electrophysiology-related genes shown here are selected from the differentiation-related gene sets identified by RRR models. The values are normalized to 0 to 1 for each feature (FIG. 14E) and gene (FIG. 14F), respectively. FIG. 14G shows that in situ electro-seq enables the multimodal spatial mapping of the cardiac tissue with heterogeneous cell populations. FIG. 14H provides a zoomed-in image of the white box highlighted region in (FIG. 14G) showing the representative region that contains multiple spatially arranged cell populations with distinct electrophysiological activities (inset). FIG. 14I shows that 25 representative regions mapped by in situ electro-seq highlight spatially-solved cell types and electrophysiological waveforms from the arrow-labeled regions in FIG. 14G. Different t-states and e-states for cells are labeled according to the legend provided in the top left. Numbers indicate E-barcoded electrode channels.

FIGS. 15A-15G show the design and fabrication of stretchable mesh electronics. FIG. 15A provides a top-view schematic showing the structure of stretchable mesh electronics. FIG. 15B provides an exploded-view schematic showing functional layers: Bottom SU-8 passivation layer; Au interconnects; Pt black-coated electrodes; top SU-8 passivation; and Electronic barcodes (E-barcodes). FIG. 15C provides schematics showing the key steps of fabrication flow of stretchable mesh electronics: (i) Deposition of 100-nm-thick Ni as the sacrificial layer on the glass substrate; (ii) Pattern 400-nm-thick SU-8 as the bottom passivation layer; (iii) Pattern 40-nm-thick Au as the interconnects; (iv) Pattern 50-nm-thick Pt as the electrodes; (v) Pattern 400-nm-thick SU-8 as the top passivation layer; and (vi) Pattern 400-nm-thick SU-8 doped with Rhodamine 6G as E-barcodes. FIG. 15D shows a photograph of 9 64-channel stretchable mesh electronics fabricated on a 4-inch soda lime glass wafer. FIG. 15E shows a bright-field (BF) optical image of an electrode array in stretchable mesh electronics on the substrate. FIG. 15F shows a BF optical image of a representative electrode with the paired E-barcodes and interconnects. FIG. 15G shows a representative design of a binary barcode for labeling 64 electrodes.

FIGS. 16A-16E show a 3D cardiac tissue-electronics hybrid. FIG. 16A provides schematics showing the protocol for cardiac differentiation from human induced pluripotent stem cells (hiPSCs, IMR90-1) by canonical Wnt pathway signaling modulation with CHIR99021 and IWR1. The hiPSC-CMs will be dissociated into single cells and integrated with stretchable mesh electronics to form electronics-embedded cardiac patches. FIG. 16B provides bright-field (BF) phase images show the cell morphology of Day 0, Day 1, Day 3, Day 5, and Day 7 of differentiation. FIG. 16C provides photographs showing the side view (top) and top view (bottom) of the cell culture chamber bonded on the glass substrate with stretchable mesh electronics. FIG. 16D provides photographs showing a hiPSC-CM tissue-electronics hybrid in the cell culture chamber. The dashed line highlights the boundary of the hiPSC-CM tissue-electronics hybrid (left). Zoom-in BF image shows the cardiac tissue and stretchable mesh electronics from the box (right). FIG. 16E is a BF phase image showing the hiPSC-CM tissue-electronics hybrid (left). Zoom-in image highlights the interaction between hiPSC-CM tissue and stretchable mesh electronics (right).

FIGS. 17A-17B show 3D in situ electro-seq of hiPSC-CM tissues and locating electrically recorded CMs. FIG. 17A shows six representative raw fluorescence images of five cycles sequencing and one cycle E-barcode imaging at Day 46. Ch, code for the four fluorescence channels as shown in the top left; Electrodes were imaged using reflection mode and highlighted as grey; E-barcodes were labeled with the fluorescent dye Rhodamine G (R6G); DAPI, cell nuclei staining. FIG. 17B provides schematics showing the process of finding electrically recorded CM. A bright field image was projected to the x-y plane and transferred to a gray-scale image, which was thresholded, gaussian filtered, and dilated to find the electrode mask in the x-y plane. The electrode mask in 3D space was fitted as a linear 2D surface. The electrically recorded cell was identified as the cell having the most intersection with the electrode mask in 3D space.

FIGS. 18A-18C show electrophysiological mapping of hiPSC-CM tissues over the time course of in vitro maturation. FIG. 18A provides photographs showing the multiplexing recording setup that measures hiPSC-CM tissues maturation. FIG. 18B shows the representative 64-channel voltage traces recorded from the hiPSC-CM tissues at Day 12, Day 21, Day 46, and Day 64 of differentiation, respectively. FIG. 18C shows the representative single-spike action potential recordings at Day 12, Day 21, Day 46, and Day 64 of differentiation, respectively.

FIGS. 19A-19B provide a comparison of cell segmentation performance by the ClusterMap and StarDist methods. FIG. 19A provides box plots showing the total RNA counts per cell in each position of cardiac tissues at Day 12, Day 21, Day 46, and Day 64 of differentiation, respectively, using the ClusterMap and StarDist cell segmentation methods. 72 positions were imaged and analyzed for each sample. FIG. 19B provides violin plots showing the distribution of total RNA counts and gene counts per cell at Day 12, Day 21, Day 46, and Day 64 of differentiation, respectively, using ClusterMap and StarDist cell segmentation methods. The results show that ClusterMap can identify more RNA counts per cell from the samples compared with the StarDist method.

FIGS. 20A-20P provide a comparison of cell typing analysis by ClusterMap and StarDist methods. Cell typing information in FIGS. 20A-20H and FIGS. 20I-20P was from ClusterMap- and StarDist-based cell segmentations, respectively. FIG. 20A and FIG. 20I show that UMAP visualizations of all the cells from electronics-embedded cardiac tissues and control cardiac tissues (without electronics embedding) show a similar distribution, suggesting negligible effects from electronics embedding on gene expression. FIG. 20B and FIG. 20J show that UMAP visualizations of all the cells from cardiac electronics-embedded cardiac tissues and control cardiac tissues (without electronics embedding) labeled by the different days of differentiation show similar cluster distributions, suggesting negligible effects from electronics embedding on gene expression over maturation. Symbols correspond to days of differentiation. FIG. 20C and FIG. 20K show that UMAP visualizations highlight the cell types clustered by Leiden clustering and their distributions at Day 12, Day 21, Day 46, and Day 64 of differentiation, respectively. Shading corresponds to different cell types. FIG. 20D and FIG. 20L show heatmaps of the top 42 differentially expressed genes aligned with each cell type. Normalized gene expression value is labeled as shown in the legend on the bottom right. The values are normalized to 0 to 1 for each gene. FIG. 20E and 20M show dot plots of selected marker gene expression in cardiac tissues. The size of the dot corresponds to the percentage of cells within a cell type, and its shading corresponds to the average expression level. FIG. 20F and FIG. 20N provide UMAP visualizations showing the trajectory of the cardiac tissue maturation using only CM cells (top). Days of differentiation are labeled as shown in the legend on the right. The line corresponds to the principal graph learned by Monocle 3. A stacked bar plot is also provided showing the percentage of cells across inferred pseudotime of cardiac tissue development (middle). Days of differentiation are labeled as shown in the legend on the right. UMAP visualizations showing the trajectory of cardiac tissue maturation are also provided (bottom). Inferred pseudotime is labeled as shown in the legend on the right. The line corresponds to the principal graph learned by Monocle 3. The trajectory starting anchor was manually chosen on the graph position of Day 12 as the start of the pseudotime. FIG. 20G and FIG. 20O are kinetics plots showing the relative expression of cardiac lineage marker genes across maturation pseudotime. Days of differentiation are labeled as shown in the legend at the top. FIG. 20H and FIG. 20P are gene ontology analyses showing significant terms related to cardiac muscle contraction, conduction, and development. Bar plot displays the top 10 significant (FDR<0.05) gene ontology (GO) terms enriched in electrophysiological related genes, mostly involved in cardiac muscle contraction, conduction, and development.

FIGS. 21A-21C show the process of locating and clustering results of electrically recorded CMs by in situ electro-seq. FIG. 21A provides a heatmap for the top 34 differentially expressed genes aligned with each cell cluster. The values are normalized to 0 to 1 for each gene. FIG. 21B shows UMAP visualizations highlighting the cell types clustered by Leiden clustering. Symbols correspond to different cell types. FIG. 21C shows UMAP visualization showing CM cell type-related gene expression across all the electrically recorded CMs. Shading corresponds to z-scored expression level.

FIGS. 22A-22F show cross-modal visualization and correlation of in situ electro-seq results of hiPSC-CM maturation by reduced-rank regression (RRR) analysis. FIG. 22A shows test R² of ‘relaxed’ and ‘naive’ sparse RRR for ion channel related genes with α=0.25, 0.5, 0.75, and 1, respectively. FIG. 22B shows cross-validated correlations of ‘relaxed’ and ‘naive’ sparse RRR for ion channel related genes with α=0.25, 0.5, 0.75, and 1, respectively. FIG. 22C shows test R² of ‘relaxed’ and ‘naive’ sparse RRR for 97 CM-related top differentially expressed genes with α=0.25, 0.5, 0.75, and 1, respectively. FIG. 22D shows cross-validated correlations of ‘relaxed’ and ‘naive’ sparse RRR for 97 CM-related differentially expressed genes with α=0.25, 0.5, 0.75, and 1, respectively. FIG. 22E shows that 62 representative electrophysiological features are extracted through down sampling operations for each spike waveform on Day 12, Day 21, Day 46, and Day 64 of differentiation, respectively. 1.6-second waveforms are sampled to 20 bins. Inset: 0.15-second fast spikes are sampled to 42 bins. FIG. 22F shows a sparse RRR model to visualize and align t-states and e-states. Components 1 and 2 of the rank-5 model are shown, n=162. The model selects the 97 and 32 most related genes to train the model and visualize, respectively.

FIGS. 23A-23L show cross-modal prediction of in situ electro-seq results of hiPSC-CM maturation by coupled autoencoder. FIGS. 23A-23B show UMAP visualization of the extracted features of single spike waveforms from the continuous electrical recording of one hiPSC-CM patch shaded by days of differentiation (FIG. 23A) and pseudotime by Monocle3 normalized to 0-1 (FIG. 23B). FIGS. 23C-23D provide distribution plots showing pseudotime distributions by Monocle3 using electrophysiology, shaded by days of differentiation. FIG. 23E provides coupled autoencoder-encoded 2D representations trained by in situ electro-seq data showing the distribution of transcriptional (Zt) and electrophysiological (Ze) data. Shading corresponds to days of differentiation. FIG. 23F provides a coupled autoencoder-encoded 2D representation showing cross-modal predicted gene expressions (Ze-t) from continuous electrical recording using the trained coupled autoencoder. FIGS. 23G-23H provide distribution plots showing pseudotime distributions by Monocle3 using electrophysiology shaded by days of differentiation for the 3D cardiac organoid. FIG. 23I provides a coupled autoencoder-encoded 2D representation showing continuous electrical recording (Ze) using the trained coupled autoencoder for the 3D cardiac organoid. FIG. 23J provides a coupled autoencoder-encoded 2D representation showing cross-modal predicted gene expressions (Ze-t) for the 3D cardiac organoid. FIG. 23K provides a heatmap showing single-cell electrophysiological features predicted for the 3D cardiac organoid. The values are normalized to 0 to 1 for each feature. FIG. 23L provides a heatmap showing single cell resolution gene expression predicted for the 3D cardiac organoid. Thirteen (13) genes were selected according to the RRR plot results. The values are normalized to 0 to 1 for each gene.

FIGS. 24A-24C show in situ electro-seq of cardiac tissue with heterogeneous cardiomyocyte CM types. FIG. 24A shows raw fluorescence images of five cycles of in situ sequencing and one cycle of barcode imaging. Ch, shaded for the four fluorescence channels as shown in the legend in the top left. Electrodes were imaged using reflection mode and shaded as grey. DAPI, cell nuclei staining. FIG. 24B provides UMAP visualizations of all the cells from the heterogeneous cardiac tissue showing three types of cells: fibroblast (Fib), cardiomyocytes 1 (CM1), and cardiomyocytes 2 (CM 2). FIG. 24C provides a heatmap of the 28 top differentially expressed genes aligned with each cell type. Shading corresponds to normalized expression value as shown in the legend in the bottom right.

FIGS. 25A-25C show application of the in situ electro-seq method to the neural system. FIG. 25A shows raw fluorescence imaging of in-process in situ electro-seq for >1000 genes with a full view of the entire neural tissue-electronics hybrid. FIG. 25B shows 3D neuron identification by spike detection from a multiple electrode array. The identified neurons and corresponding electrodes are shown with recorded single-unit action potential overlapped on the image. FIG. 25C provides a heatmap showing the extracted features from the waveform of averaged spikes and corresponding highest differentially expressed genes expressed in the electrically recorded neurons. Normalized electrophysiological feature value (left) and gene expression value (right) are shown.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology(2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, “electrophysiological activity” refers to the electrical properties of a biological system. In some embodiments, the biological system is a cell, or multiple cells. In some embodiments, the biological system is a tissue. In some embodiments, the biological system is an organ. In certain embodiments, the biological system is a subject. In some embodiments, the subject is a mammal (e.g., a human). The electrophysiological activity of a biological system involves measurements of voltage changes or electric current, and in particular the flow of ions. This includes the electrical activity of neurons (action potential activity), the heart (e.g., cardiomyocytes), and the brain.

As used herein, a “tissue” is a group of cells and their extracellular matrix from the same origin. Together, the cells carry out a specific function. The association of multiple tissue types together forms an organ. The cells may be of different types. In some embodiments, a tissue is an epithelial tissue. Epithelial tissues are formed by cells that cover organ surface (e.g., the surface of the skin, airways, soft organs, reproductive tract, and inner lining of the digestive tract). Epithelial tissues perform protective functions and are also involved in secretion, excretion, and absorption. Examples of epithelial tissues include, but are not limited to, simple squamous epithelium, stratified squamous epithelium, simple cuboidal epithelium, transitional epithelium, pseudostratified epithelium, columnar epithelium, and glandular epithelium. In some embodiments, a tissue is a connective tissue. Connective tissues are fibrous tissues made up of cells separated by non-living material (e.g., an extracellular matrix). Connective tissues provide shape to organs and hold organs in place. Connective tissues include fibrous connective tissue, skeletal connective tissue, and fluid connective tissue. Examples of connective tissues include, but are not limited to, blood, bone, tendon, ligament, adipose, and areolar tissues. In some embodiments, a tissue is a muscular tissue. Muscular tissue is an active contractile tissue formed from muscle cells. Muscle tissue functions to produce force and cause motion. Muscle tissue includes smooth muscle (e.g., as found in the inner linings of organs), skeletal muscle (e.g., as typically attached to bones), and cardiac muscle (e.g., as found in the heart, where it contracts to pump blood throughout an organism). In some embodiments, a tissue is a nervous tissue. Nervous tissue includes cells comprising the central nervous system and peripheral nervous system. Nervous tissue forms the brain, spinal cord, cranial nerves, and spinal nerves (e.g., motor neurons). In certain embodiments, a tissue is brain tissue. In certain embodiments, a tissue is heart tissue. In certain embodiments, a tissue is pancreas tissue. In certain embodiments, a tissue is developing tissue. In some embodiments, a tissue is any tissue with a physiological signal that can be detected by an electrical sensor. In some embodiments, a tissue is any tissue that generates or responds to an electrical signal. In some embodiments a tissue comprises skin, muscle, cardiac muscle, GI tract tissue, smooth muscle, skeletal muscle, pancreatic tissue, central nervous system tissue, nerves, glands, breast tissue, uterine tissue, or bladder tissue.

A tissue may also be a “diseased tissue.” A diseased tissue is a tissue sample taken from a subject who has been diagnosed with or is thought to have a disease (e.g., any of the diseases disclosed herein). Diseased tissue samples may be obtained from subjects diagnosed with or thought to have a neuropsychiatric disease (e.g., autism spectrum disorder, bipolar disorders, etc.), a cardiac disease, such as cardiac arrhythmia (e.g., atrial fibrillation, ventricular tachycardia, etc.), or any other disease described herein. In some embodiments, a diseased tissue comprises skin (e.g., from a subject with a dermatologic disease). In some embodiments, a diseased tissue comprises a tumor (e.g., from a subject with cancer).

As used herein, a “nanoelectronic device” is a nanoscale wire or other device small enough as to be injectable or insertable into a biological tissue. In some cases, the device may comprise one or more nanoscale wires. Other components, such as fluids or cells, may also be injected or inserted. In addition, in some cases, the nanoelectronic device, after insertion or injection, may be connected to an external electrical circuit, e.g., to a computer. The nanoelectronic device may be used to determine a property of the tissue or other matter, and/or provide an electrical signal to the tissue or other matter. This may be achieved using one or more nanoscale wires on the nanoelectronic device. In some cases, at least one of the nanoscale wires is a silicon nanowire. In certain embodiments, a nanoelectronic device comprising nanoscale wires may be inserted into an electrically-active tissue, such as the heart or the brain, and the nanoscale wires may be used to determine electrical properties of the tissue, e.g., action potentials or other electrical activity. In some embodiments, the nanoelectronic device is relatively porous to allow cells, etc. to grow or migrate into the nanoelectronic device. For example, neurons may grow into the nanoelectronic device. This may be useful, for example, for long-term applications (e.g., where the nanoelectronic device is to be inserted and used for days, weeks, months, or years within the tissue). For example, neurons or cardiac cells may be able to grow around and/or into the nanoelectronic device while it is inserted into, for example, the brain or the heart, e.g., over extended periods of time.

In some embodiments, a nanoelectronic device may be formed from one or more polymeric constructs and/or metal leads. In some embodiments, the nanoelectronic device is relatively small and may include components, such as nanoscale wires. The device may also be flexible and/or have a relatively open structure, e.g., an open porosity of at least about 30%, or other porosities. For instance, the nanoelectronic device may be formed from a plurality of nanoscale wires, connected by polymeric constructs and/or metal leads, forming a relatively large or open network, which can then be rolled to form a cylindrical or other 3-dimensional structure that is to be inserted into a subject. In some embodiments, the nanoscale wires may be distributed about the nanoelectronic device, e.g., in three dimensions, thereby allowing determination of properties and/or stimulation of a tissue, etc. in three-dimensions. The nanoelectronic device can also be connected to an external electrical system, e.g., to facilitate use of the nanoelectronic device.

In certain aspects, the nanoelectronic device may comprise one or more electrical networks comprising nanoscale wires and conductive pathways in electrical communication with the nanoscale wires. In some embodiments, at least some of the conductive pathways may also provide mechanical strength to the nanoelectronic device, and/or there may be polymeric or metal constructs that are used to provide mechanical strength to the nanoelectronic device. The nanoelectronic device may be planar or substantially define a plane, or the device may be non-planar or curved (i.e., a surface that can be characterized as having a finite radius of curvature). The nanoelectronic device may also be flexible in some cases, e.g., the device may be able to bend or flex. For example, a device may be bent or distorted by a volumetric displacement of at least about 5%, about 10%, or about 20% (relative to the undisturbed volume), without causing cracks and/or breakage within the nanoelectronic device. For example, in some cases, the nanoelectronic device can be distorted such that about 5%, about 10%, or about 20% of the mass of the nanoelectronic device has been moved outside the original surface perimeter of the device, without causing failure of the device (e.g., by breaking or cracking of the device, disconnection of portions of the electrical network, etc.). In some embodiments, the nanoelectronic device may be bent or flexed as described above by an ordinary human being without the use of tools, machines, mechanical device, excessive force, or the like. A flexible nanoelectronic device may be more biocompatible due to its flexibility.

The nanoelectronic device may comprise a biocompatible material. As used herein, a biocompatible material is one that does not illicit an immune response, or elicits a relatively low immune response, e.g., one that does not impair the device or the cells therein from continuing to function for its intended use. In some embodiments, the biocompatible material is able to perform its desired function without eliciting any undesirable local or systemic effects in the subject. In some cases, the material can be incorporated into tissues within the subject, e.g., without eliciting any undesirable local or systemic effects, or such that any biological response by the subject does not substantially affect the ability of the material from continuing to function for its intended use. For example, in a device, the device may be able to determine cellular or tissue activity after insertion, e.g., without substantially eliciting undesirable effects in those cells, or undesirable local or systemic responses, or without eliciting a response that causes the device to cease functioning for its intended use. Examples of techniques for determining biocompatibility include, but are not limited to, the ISO 10993 series for evaluating the biocompatibility of medical devices. As another example, a biocompatible material may be implanted in a subject for an extended period of time, e.g., at least about a month, at least about 6 months, or at least about a year, and the integrity of the material, or the immune response to the material, may be determined. For example, a suitably biocompatible material may be one in which the immune response is minimal, e.g., one that does not substantially harm the health of the subject. Example of biocompatible materials include, but are not limited to, poly(methyl methacrylate), polyvinylchloride, polyethylene, polypropylene, polystyrene, polytetrafluoroethylene, polyurethane, polyamide, polyethylenterephthalate, and polyethersulfone. In some embodiments, a biocompatible material may be used to cover or shield a non-biocompatible material (or a poorly biocompatible material) from the cells or tissue, for example, by covering the material.

In certain embodiments, the nanoelectronic device comprises a unique identification tag. In some embodiments, the tag is an electronic barcode. In some embodiments, the electronic barcode is a fluorescence electronic barcode. The fluorescence electronic barcode on each nanoelectronic device is unique to that particular device. The barcode allows identification of the location of a particular nanoelectronic device within a system (e.g., within a particular cell within a tissue). In some embodiments, the location of the nanoelectronic device is determined by microscopy (e.g., confocal microscopy).

In some embodiments, a nanoelectronic device comprises an actuator. As used herein, an “actuator” is a component of a machine that moves and controls the system to perform an operation or task. Actuators include, but are not limited to, hydraulic actuators, pneumatic actuators, electric actuators, thermal actuators, magnetic actuators, and mechanical actuators.

As used herein, “electrophysiological recording” refers to methods that enable measurement of electrophysiological activity, as described above. In some instances, electrophysiological recording involves placing electrodes (e.g., simple solid conductors, tracings on printed circuit board or flexible polymers, or hollow tubes filled with an electrolyte) into various preparations of biological tissue (e.g., living organisms, excised tissue, dissociated cells from excised tissue, or artificially grown cells or tissues). In some embodiments, electrophysiological recording comprises intracellular electrophysiological recording. Intracellular electrophysiological recording comprises measuring voltage or current across the membrane of a cell. Methods for performing intracellular recording include, but are not limited to, methods utilizing a voltage clamp (Hernandez-Ochoa, E. O.; Schneider, M. F. Prog. Biophys. Mol. Biol. 2012, 108(3), 98-118), methods utilizing a current clamp (Bolanos-Burgos, I. V. et al. Cerebellum. 2020), patch-clamp recording (Schlegel, A. M., et al. Channels. 2020, 14(1), 310-325), and sharp electrode recording (Gao, L., Wang, X., Nature Protoc. 2020). In some embodiments, electrophysiological recording is performed in a tissue (e.g., any of the tissues described herein).

As used herein, “gene expression” refers to the process by which information from a gene is used in the synthesis of a gene product. Gene products include proteins and RNA (e.g., messenger RNA, transfer RNA, or small nuclear RNA). Gene expression includes transcription and translation. Transcription is the process by which a segment of DNA is transcribed into RNA by an RNA polymerase. Translation is the process by which an RNA is translated into a peptide or protein by a ribosome.

As used herein, “in situ single-cell transcriptome sequencing” refers to methods for analyzing the gene expression of single cells within a large population of cells. In certain embodiments, in situ single-cell transcriptome sequencing is in situ single-cell RNA sequencing. Such methods provide the expression profiles of individual cells, allowing patterns of gene expression to be identified through gene clustering analyses. Methods for in situ single-cell transcriptome sequencing or in situ single-cell RNA sequencing include isolating single cells and their RNA, followed by reverse transcription, amplification, library generation, and sequencing. In some embodiments, individual cells are separated into separate wells. In some embodiments, individual cells are encapsulated in droplets in a microfluidic device, wherein each droplet carries a unique gene-specific identifier sequence, allowing nucleic acids from various cells to be mixed together for sequencing and transcripts from individual cells identified afterward.

In some embodiments, in situ single-cell transcriptome sequencing comprises spatially-resolved transcript amplicon readout mapping (STAR-map) (Wang, X., et al. 2018, 361, 180; WO 2019/199579). The STAR-map method is a method for in situ gene sequencing of a target nucleic acid in a cell in an intact tissue comprising: (a) contacting a fixed and permeabilized intact tissue with at least a pair of oligonucleotide primers under conditions to allow for specific hybridization; (b) adding ligase to ligate the second oligonucleotide and generate a closed nucleic acid circle; (c) performing rolling circle amplification; (d) embedding one or more amplicons in the presence of hydrogel subunits to form one or more hydrogel-embedded amplicons; (e) contacting the one or more hydrogel-embedded amplicons with a pair of primers under conditions to allow for ligation; (f) reiterating step (e); and (g) imaging the one or more hydrogel-embedded amplicons to determine in situ gene sequencing of the target nucleic acid in the cell in the intact tissue. In certain embodiments, the primers used in STAR-map comprise gene-specific identifier sequences, allowing for transcriptomic mapping of gene expression within a cell.

As used herein, an “organoid” refers to a miniaturized and simplified version of an organ produced in vitro in three dimensions. In certain embodiments, organoids are derived from one or a few cells from a tissue. In certain embodiments, organoids are derived from embryonic stem cells. In certain embodiments, organoids are derived from induced pluripotent stem cells. Organoids include, but are not limited to, cerebral organoids (e.g., organoids resembling the brain), gut organoids (e.g., organoids resembling structures of the gastrointestinal tract), thyroid organoids, thymic organoids, testicular organoids, hepatic organoids, pancreatic organoids, epithelial organoids, lung organoids, kidney organoids, embryonic organoids, cardiac organoids, and retinal organoids.

The term “neurological disease” refers to any disease of the nervous system, including diseases that involve the central nervous system (brain, brainstem, and cerebellum), the peripheral nervous system (including cranial nerves), and the autonomic nervous system (parts of which are located in both central and peripheral nervous system). Also included are any diseases affecting the nervous tissue of any organ, including the eye or the retina of the eye. Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The term “cardiovascular disease” or “cardiovascular disorder” refers to any disease or disorder involving the heart or blood vessels. Diseases and disorders including, but not limited to, stroke, heart failure, hypertension, cardiomyopathy, arrhythmias (e.g., atrial fibrillation, ventricular tachycardia, junctional arrhythmia, heart blocks, sudden arrhythmic death syndrome, fetal arrhythmia, etc.)., and coronary artery diseases (e.g., angina and myocardial infarction) are all encompassed by this term.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

The present disclosure provides methods for correlating a continuous physiological process (e.g., electrophysiological activity) and a biomolecular process (e.g., gene expression) in multiple cells within a tissue. Further disclosed herein are systems comprising nanoelectronic devices in cells in a tissue, wherein each nanoelectronic device comprises a unique electronic barcode. Also, disclosed herein are methods for preparing a tissue for continuous electrophysiological recording. Additionally disclosed herein are methods for discovering a target for treating a disease and methods for drug screening.

### Methods for Correlating A Physiological Process and A Biomolecular Process in a Cell

The present disclosure provides methods for correlating a continuous physiological process and a biomolecular process in cells in a tissue, the method comprising steps of:
(a) embedding one or more nanoelectronic devices in the tissue to form a nanoelectronics-tissue hybrid, wherein each nanoelectronic device comprises at least one sensor with a unique electronic barcode;
(b) performing a continuous physiological measurement on the cells;
(c) fixing the nanoelectronics-tissue hybrid;
(d) performing in situ analysis of the biomolecular process on the cells;
(e) performing mapping of the biomolecular process on the cells;
(f) identifying the position of the electronic barcode within the nanoelectronics-tissue hybrid; and
(g) performing cell segmentation to correlate the mapping of the in situ analysis of the biomolecular process with the continuous physiological measurement.

Any continuous physiological process can be studied using the methods disclosed herein. In some embodiments, the continuous physiological process is an electrical process, a mechanical process, or a chemical process. In certain embodiments, the continuous physiological process comprises electrophysiological activity (e.g., the electrophysiological activity of cells in a tissue). In some embodiments, the step of performing a continuous physiological measurement comprises performing continuous electrophysiological recording.

The present disclosure also contemplates studying any biomolecular process using the methods disclosed herein. In some embodiments, the biomolecular process is DNA replication, DNA translation, RNA transcription, gene expression, or protein expression. In certain embodiments, the biomolecular process comprises gene expression (e.g., gene expression in cells in a tissue). In some embodiments, the step of performing in situ analysis of the biomolecular process comprises performing in situ single-cell transcriptome sequencing. In certain embodiments, the step of performing mapping of the biomolecular process comprises performing transcriptomic mapping.

The present disclosure also provides methods for correlating electrophysiological activity and gene expression in cells in a tissue. In some embodiments, the method comprises the steps of:
(a) embedding one or more nanoelectronic devices in the tissue to form a nanoelectronics-tissue hybrid, wherein each nanoelectronic device comprises at least one sensor with a unique electronic barcode;
(b) performing continuous electrophysiological recording on the cells;
(c) fixing the nanoelectronics-tissue hybrid;
(d) performing in situ single-cell transcriptome sequencing on the cells;
(e) performing transcriptomic mapping on the cells;
(f) identifying the position of the electronic barcode within the nanoelectronics-tissue hybrid; and
(g) performing cell segmentation to correlate the single-cell transcriptome sequencing data with the electrophysiological recording data.

In the methods described herein, the step of continuous electrophysiological recording may be performed for various lengths of time. In some embodiments, the step of continuous electrophysiological recording is performed for an extended period of time. In certain embodiments, the step of continuous electrophysiological recording is performed for at least 1 day. In certain embodiments, the step of continuous electrophysiological recording is performed for at least 10 days. In certain embodiments, the step of continuous electrophysiological recording is performed for at least 1 month, at least 2 months, at least 3 months, at least 4 months, at least 5 months, at least 6 months, at least 7 months, at least 8 months, at least 9 months, at least 10 months, or at least 11 months. In certain embodiments, the step of continuous electrophysiological recording is performed for more than 1 year. The step of continuous electrophysiological recording may also be performed for shorter periods of time (e.g., hours, minutes, or seconds). In some embodiments, the step of continuous electrophysiological recording is performed for at least 1 minute, at least 10 minutes, at least 20 minutes, at least 30 minutes, at least 40 minutes, at least 50 minutes, or at least 60 minutes. In certain embodiments, the step of continuous electrophysiological recording is performed for at least 1 hour, at least 2 hours, at least 3 hours, at least 4 hours, at least 5 hours, at least 6 hours, at least 12 hours, at least 18 hours, or at least 24 hours.

Varying numbers of nanoelectronic devices and sensors may be used in the methods disclosed herein. Factors that may affect the number of nanoelectronic devices and sensors used include, but are not limited to, the size of the tissue being studied, the type of tissue being studied, and the number of cells in the tissue being studied. In certain embodiments, the nanoelectronic devices embedded within the tissue comprise over 100, over 200, over 300, over 400, over 500, over 600, over 700, over 800, over 900, or over 1000 sensors. In certain embodiments, the nanoelectronic devices embedded within the tissue comprise over 2000, over 3000, over 4000, over 5000, over 6000, over 7000, over 8000, over 9000, over 10,000, over 20,000, over 30,000, over 40,000, over 50,000, over 60,000, over 70,000, over 80,000, over 90,000, over 100,000, over 200,000, over 300,000, over 400,000, over 500,000, over 600,000, over 700,000, over 800,000, over 900,000, or over 1,000,000 sensors. In some embodiments, the tissue comprises over 1,000,000 cells. In some embodiments, the tissue comprises over 1,000,000,000 cells.

Each nanoelectronic device used in the methods described herein comprises at least one sensor comprising a unique electronic barcode. In some embodiments, the electronic barcode is a fluorescence electronic barcode. In some embodiments, the electronic barcode is a photodiode barcode. In some embodiments, the electronic barcode is a transistor barcode. In certain embodiments, each electronic barcode comprises a unique binary code. The electronic barcodes utilized in the presently described methods may be read out using various methods known in the art including, but not limited to, any type of fluorescence imaging (e.g., microscopy). In some embodiments, the electronic barcodes are read out by confocal microscopy. In certain embodiments, multiple rounds of fluorescence imaging are performed to read out all of the electronic barcodes present in a sample. In certain embodiments, the step of transcriptomic mapping is performed through fluorescence imaging simultaneously with the step of identifying the position of the electronic barcode within the tissue sample.

The present disclosure contemplates the use of various nanoelectronic devices in the methods described herein. In some embodiments, the nanoelectronic devices are tissue-like (e.g., nanoelectronic sensing units are embedded into a flexible, mesh-like network capable of forming seamless integration with 3D tissue networks). In some embodiments, the nanoelectronic devices comprise a polymeric network. In certain embodiments, the nanoelectronic devices comprise a stretchable mesh. In some embodiments, the stretchable mesh comprises an overall filling ratio of less than 100%, less than 50%, less than 20%, less than 15%, less than 10%, or less than 5%. In certain embodiments, the stretchable mesh comprises an overall filling ratio of less than 11%.

In some embodiments, the nanoelectronic device is embedded in a serpentine layout throughout the tissue (e.g., the devices snake back-and-forth, parallel to one another, throughout the tissue). In some embodiments, the nanoelectronic device is embedded in a hexagonal layout, a triangular layout, or a straight layout. In certain embodiments, the nanoelectronic devices comprise a mass of less than 50 μg, less than 40 μg, less than 30 μg, less than 20 μg, or less than less than 10 μg. In certain embodiments, the nanoelectronic device comprises a mass of less than 15 μg. In some embodiments, the nanoelectronic devices comprise a top encapsulation layer. The top encapsulation layer of each nanoelectronic device may comprise an epoxy-based material. In some embodiments, the epoxy-based material may be a photoresist (e.g., SU-8, S1805, LOR 3A, poly(methylmethacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, and Shipley 1400-37). Many other photoresists are commercially available and well-known in the art. In some embodiments, the top encapsulation layer is an SU-8 encapsulation layer (i.e., the top encapsulation layer comprises the epoxy-based material SU-8). The nanoelectronic devices used in the methods described herein may further comprise an electrode layer. In some embodiments, the electrode layer is a platinum, graphite, copper, titanium, silver, palladium, or mixed metal oxide electrode layer (e.g., comprising RuO₂, IrO₂, PtO₂, and/or TiO₂). In certain embodiments, the electrode layer comprises a coating (e.g., a coating comprising a metal, organic material, mineral, and/or binder). In some embodiments, the coating is a poly(3,4-ethylenedioxythiophene) coating, a polyaniline coating, or a polypyrrole coating. In certain embodiments, the coating is a poly(3,4-ethylenedioxythiophene) coating. In some embodiments, the nanoelectronic devices comprise a gold, silver, copper, or other metal interconnecting layer. In some embodiments, the nanoelectronic devices comprise a bottom encapsulation layer. The bottom encapsulation layer of each nanoelectronic device may comprise an epoxy-based material. In some embodiments, the epoxy-based material may be a photoresist (e.g., SU-8, S1805, LOR 3A, poly(methylmethacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, and Shipley 1400-37). Many other photoresists are commercially available and well-known in the art. In certain embodiments, the bottom encapsulation layer is an SU-8 encapsulation layer. In certain embodiments, the nanoelectronic devices comprise input and output lines. In some embodiments, the nanoelectronic devices comprise an electrical device. In some embodiments, the nanoelectronic devices comprise an optical device. In some embodiments, the nanoelectronic devices comprise a mechanical sensor. In some embodiments, the nanoelectronic devices comprise a stimulator. In some embodiments, the nanoelectronic devices comprise an actuator.

In certain embodiments, the step of embedding the nanoelectronic devices comprises transferring the nanoelectronic devices onto a two-dimensional sheet of cells and allowing the cells to aggregate, associate, proliferate, differentiate, and/or migrate. In some embodiments, allowing the cells to aggregate, associate, proliferate, and/or migrate compresses the nanoelectronic devices and embeds them within the cells in the tissue.

Various methods for the step of performing in situ single cell transcriptome sequencing are contemplated by the present disclosure, including, but not limited to, those described in Wang, X., et al. 2018, 361, 180 and International Patent Application Publication No. WO 2019/199579, which are incorporate herein by reference. In some embodiments, the step of performing in situ single cell transcriptome sequencing comprises constructing cDNA amplicons in situ by probe hybridization. In some embodiments, the step of performing in situ single cell transcriptome sequencing comprises enzymatic amplification of the cDNA amplicons. In some embodiments, the step of performing in situ single cell transcriptome sequencing comprises immobilization of the amplified cDNA in a hydrogel network. In certain embodiments, the cDNA amplicons comprise a gene-specific identifier sequence. In some embodiments, the gene-specific identifier sequence is read out through imaging. In certain embodiments, the imaging is fluorescent imaging. In some embodiments, the gene-specific identifier sequence is read out by microscopy (e.g., confocal microscopy). The gene-specific identifier sequence may be read out simultaneously with the step of identifying the position of the electronic barcodes within the tissue sample.

In some embodiments, the step of performing in situ single cell transcriptome sequencing comprises performing single cell RNA sequencing. In certain embodiments, the step of performing in situ single cell transcriptome sequencing comprises performing spatially-resolved transcript amplicon readout mapping (STARmap). In some embodiments, the step of transcriptomic mapping comprises mapping over 25, over 50, over 100, over 200, over 300, over 400, over 500, over 600, over 700, over 800, over 900, or over 1000 genes simultaneously. In some embodiments, the step of identifying the position of the fluorescence electronic barcode comprises performing confocal microscopy.

The use of various tissues in the methods described herein is contemplated by the present disclosure. In some embodiments, a tissue is any tissue with a physiological signal that can be detected by an electrical sensor. The tissues may comprise various cells and cell types. In some embodiments, the cell is living. In certain embodiments, the cell is in vivo. In some embodiments, the tissue is living. In some embodiments, the tissue is in vivo. In certain embodiments, the tissue is three-dimensional. In certain embodiments, the tissue is any tissue that has electrical activity. In certain embodiments, the tissue is brain tissue. In certain embodiments, the tissue is heart tissue. In certain embodiments, the tissue is pancreas tissue. In certain embodiments, the tissue is nervous system tissue. In certain embodiments, the tissue is muscle tissue. In certain embodiments, the tissue is gastrointestinal tract tissue. In certain embodiments, the tissue is eye or ear tissue. In certain embodiments, the tissue is adrenal gland, breast, or salivary gland tissue. In certain embodiments, the tissue is developing tissue. In some embodiments, the tissue is an organoid. In some embodiments, the tissue is human induced pluripotent stem cell-derived. In certain embodiments, the cell is a stem cell. In some embodiments, the cell is a progenitor cell. In some embodiments, a tissue comprises multiple types of cells. In some embodiments, the method comprises observing multiple types of cells in a tissue at once. The present disclosure also contemplates the use of diseased tissue in any of the methods disclosed herein (e.g., tissue samples obtained from a subject who has been diagnosed with or is otherwise thought to have a disease). In some embodiments, the diseased tissue comprises cardiac tissue (e.g., from a subject with a cardiac disease). In some embodiments, the diseased tissue comprises neurological tissue (e.g., from a subject with a neurological disease). In some embodiments, the diseased tissue comprises skin (e.g., from a subject with a dermatologic disease). In some embodiments, the diseased tissue comprises a tumor sample (e.g., from a subject with cancer).

A tissue may comprise multiple types of cells. In some embodiments, a tissue comprises epithelial cells, nerve cells, muscle cells, and/or connective tissue cells, or any combination thereof. In certain embodiments, a tissue comprises only one of these types of cells. Tissues comprising varying numbers of cells are also contemplated by the present disclosure. In some embodiments, a tissue has more than 1 million, more than 5 million, more than 10 million, more than 20 million, more than 30 million, more than 40 million, or more than 50 million cells. In certain embodiments, a tissue has more than one billion cells.

### System comprising a Nanoelectronic Device and a Cell in a Tissue

The present disclosure provides systems comprising one or more nanoelectronic devices in a cell in a tissue, wherein each nanoelectronic device comprises at least one sensor with a unique electronic barcode. The present disclosure also provides systems for correlating a continuous physiological process (e.g., electrophysiological activity) and a biomolecular process (e.g., gene expression(in cells in a tissue, wherein the system is prepared by embedding nanoelectronic devices in the tissue to form a nanoelectronics-tissue hybrid, wherein each nanoelectronic device comprises a unique electronic barcode. In some embodiments, the electronic barcode is a fluorescence electronic barcode. In some embodiments, the electronic barcode is a photodiode barcode or a transistor barcode.

Varying numbers of nanoelectronic devices and sensors may be used in the systems disclosed herein. Factors that may affect the number of nanoelectronic devices and sensors used include, but are not limited to, the size of the tissue used in the system, the type of tissue used in the system, and the number of cells in the tissue used in the system. In certain embodiments, the nanoelectronic devices embedded within the tissue comprise over 100, over 200, over 300, over 400, over 500, over 600, over 700, over 800, over 900, or over 1000 sensors. In certain embodiments, the nanoelectronic devices embedded within the tissue comprise over 2000, over 3000, over 4000, over 5000, over 6000, over 7000, over 8000, over 9000, over 10,000, over 20,000, over 30,000, over 40,000, over 50,000, over 60,000, over 70,000, over 80,000, over 90,000, over 100,000, over 200,000, over 300,000, over 400,000, over 500,000, over 600,000, over 700,000, over 800,000, over 900,000, or over 1,000,000 sensors. In some embodiments, the tissue comprises over 1,000,000 cells. In some embodiments, the tissue comprises over 1,000,000,000 cells.

The present disclosure contemplates the use of various nanoelectronic devices in the systems described herein. In some embodiments, the nanoelectronic devices are tissue-like. In some embodiments, the nanoelectronic devices comprise a polymeric network. In certain embodiments, the nanoelectronic devices comprise a stretchable mesh. In some embodiments, the stretchable mesh comprises an overall filling ratio of less than 100%, less than 50%, less than 20%, less than 15%, less than 10%, or less than 5%. In certain embodiments, the stretchable mesh comprises an overall filling ratio of less than 11%.

In some embodiments, the nanoelectronic device is embedded in a serpentine layout throughout the tissue (e.g., the devices snake back-and-forth, parallel to one another, throughout the tissue). In some embodiments, the nanoelectronic device is embedded in a hexagonal layout, a triangular layout, or a straight layout. In certain embodiments, the nanoelectronic devices comprise a mass of less than 50 μg, less than 40 μg, less than 30 μg, less than 20 μg, or less than less than 10 μg. In certain embodiments, the nanoelectronic device comprises a mass of less than 15 μg. In some embodiments, the nanoelectronic devices comprise a top encapsulation layer. The top encapsulation layer of each nanoelectronic device may comprise an epoxy-based material. In some embodiments, the epoxy-based material may be a photoresist (e.g., SU-8, S1805, LOR 3A, poly(methylmethacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, and Shipley 1400-37). Many other photoresists are commercially available and well-known in the art. In some embodiments, the top encapsulation layer is an SU-8 encapsulation layer (i.e., the top encapsulation layer comprises the epoxy-based material SU-8). The nanoelectronic devices used in the systems described herein may further comprise an electrode layer. In some embodiments, the electrode layer is a platinum, graphite, copper, titanium, silver, palladium, or mixed metal oxide electrode layer (e.g., comprising RuO₂, IrO₂, PtO₂, and/or TiO₂). In certain embodiments, the electrode layer comprises a coating (e.g., a coating comprising a metal, organic material, mineral, and/or binder). In some embodiments, the coating is a poly(3,4-ethylenedioxythiophene) coating, a polyaniline coating, or a polypyrrole coating. In certain embodiments, the coating is a poly(3,4-ethylenedioxythiophene) coating. In some embodiments, the nanoelectronic devices comprise a gold, silver, copper, or other metal interconnecting layer. In some embodiments, the nanoelectronic devices comprise a bottom encapsulation layer. The bottom encapsulation layer of each nanoelectronic device may comprise an epoxy-based material. In certain embodiments, the bottom encapsulation layer is an SU-8 encapsulation layer. In certain embodiments, the nanoelectronic devices comprise input and output lines. In some embodiments, the nanoelectronic devices comprise an electrical device. In some embodiments, the nanoelectronic devices comprise an optical device. In some embodiments, the nanoelectronic devices comprise a mechanical sensor. In some embodiments, the nanoelectronic devices comprise a stimulator. In some embodiments, the nanoelectronic devices comprise an actuator.

In certain embodiments, the nanoelectronic devices are embedded in the cells in the tissue by transferring the nanoelectronic devices onto a two-dimensional sheet of cells and allowing the cells to aggregate, associate, proliferate, differentiate and/or migrate. In some embodiments, allowing the cells to aggregate, associate, proliferate, and migrate compresses the nanoelectronic device and embeds it within the cells in the tissue.

In some embodiments, the cells are living. In certain embodiments, the cells are in vivo. In some embodiments, the tissue is living. In some embodiments, the tissue is in vivo. In certain embodiments, the tissue is three-dimensional. In some embodiments, the tissue is any tissue with electrical activity. In some embodiments, a tissue is any tissue with a physiological signal that can be detected by an electrical sensor. In some embodiments, a tissue is any tissue that generates or responds to an electrical signal. In certain embodiments, the tissue is brain tissue. In certain embodiments, the tissue is heart tissue. In certain embodiments, the tissue is pancreatic tissue. In certain embodiments, the tissue is nervous system tissue. In certain embodiments, the tissue is muscle tissue. In certain embodiments, the tissue is gastrointestinal tract tissue. In certain embodiments, the tissue is eye or ear tissue. In certain embodiments, the tissue is adrenal gland, breast, or salivary gland tissue. In certain embodiments, the tissue is developing tissue. In some embodiments, the tissue is an organoid. In some embodiments, the tissue is human induced pluripotent stem cell-derived. In certain embodiments, the cells are stem cells. In some embodiments, the cells are progenitor cells. The present disclosure also contemplates the use of diseased tissue in any of the systems disclosed herein (e.g., tissue samples obtained from a subject who has been diagnosed with or is otherwise thought to have a disease). In some embodiments, the diseased tissue comprises cardiac tissue (e.g., from a subject with a cardiac disease). In some embodiments, the diseased tissue comprises neurological tissue (e.g., from a subject with a neurological disease). In some embodiments, the diseased tissue comprises skin (e.g., from a subject with a dermatologic disease). In some embodiments, the diseased tissue comprises a tumor sample (e.g., from a subject with cancer).

A tissue may comprise multiple types of cells. In some embodiments, a tissue comprises epithelial cells, nerve cells, muscle cells, and/or connective tissue cells, or any combination thereof. In certain embodiments, a tissue comprises only one of these types of cells. Tissues comprising varying numbers of cells are also contemplated by the present disclosure. In some embodiments, a tissue has more than 1 million, more than 5 million, more than 10 million, more than 20 million, more than 30 million, more than 40 million, or more than 50 million cells. In certain embodiments, a tissue has more than one billion cells.

### Methods for Preparing a Tissue for Continuous Electrophysiological Recording

The present disclosure provides methods for preparing a tissue for continuous electrophysiological recording, the method comprising embedding one or more nanoelectronic devices in cells in the tissue to form a nanoelectronics-tissue hybrid, wherein each nanoelectronic device comprises at least one sensor with a unique electronic barcode. In some embodiments, the electronic barcode is a fluorescence electronic barcode. In some embodiments, the electronic barcode is a photodiode barcode or a transistor barcode.

In certain embodiments, the nanoelectronic devices embedded within the tissue comprise over 1000, over 10,000, over 100,000, or over 1,000,000 sensors. In some embodiments, the tissue comprises over 1,000,000 cells. In some embodiments, the tissue comprises over 1,000,000,000 cells.

In some embodiments, the nanoelectronic devices are tissue-like. In some embodiments, the nanoelectronic devices comprise a polymeric network. In certain embodiments, the nanoelectronic devices comprise stretchable mesh. In some embodiments, the stretchable mesh comprises an overall filling ratio of less 100%, less than 50%, less than 20%, less than 15%, less than 10%, or less than 5%. In certain embodiments, the stretchable mesh comprises an overall filling ratio of less than 11%.

In some embodiments, the nanoelectronic device is embedded in a serpentine layout throughout the tissue (e.g., the devices snake back-and-forth, parallel to one another, throughout the tissue). In some embodiments, the nanoelectronic device is embedded in a hexagonal layout, a triangular layout, or a straight layout. In certain embodiments, the nanoelectronic devices comprise a mass of less than 50 μg, less than 40 μg, less than 30 μg, less than 20 μg, or less than less than 10 μg. In certain embodiments, the nanoelectronic device comprises a mass of less than 15 μg. In some embodiments, the nanoelectronic devices comprise a top encapsulation layer. The top encapsulation layer of each nanoelectronic device may comprise an epoxy-based material. In some embodiments, the epoxy-based material may be a photoresist (e.g., SU-8, S1805, LOR 3A, poly(methylmethacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, and Shipley 1400-37). Many other photoresists are commercially available and well-known in the art. In some embodiments, the top encapsulation layer is an SU-8 encapsulation layer. In some embodiments, the nanoelectronic devices comprise an electrode layer. In some embodiments, the electrode layer is a platinum, graphite, copper, titanium, silver, palladium, or mixed metal oxide electrode layer (e.g., comprising RuO₂, IrO₂, PtO₂, and/or TiO₂). In certain embodiments, the electrode layer comprises a coating (e.g., a coating comprising a metal, organic material, mineral, and/or binder). In some embodiments, the coating is a poly(3,4-ethylenedioxythiophene) coating, a polyaniline coating, or a polypyrrole coating. In certain embodiments, the coating is a poly(3,4-ethylenedioxythiophene) coating. In some embodiments, the nanoelectronic devices comprise a gold, silver, copper, or other metal interconnecting layer. In some embodiments, the nanoelectronic devices comprise a bottom encapsulation layer. The bottom encapsulation layer of each nanoelectronic device may comprise an epoxy-based material. In some embodiments, the epoxy-based material may be a photoresist (e.g., SU-8, S1805, LOR 3A, poly(methylmethacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, and Shipley 1400-37). Many other photoresists are commercially available and well-known in the art. In certain embodiments, the bottom encapsulation layer is an SU-8 encapsulation layer. In certain embodiments, the nanoelectronic devices comprise input and output lines. In some embodiments, the nanoelectronic devices comprise an electrical device. In some embodiments, the nanoelectronic devices comprise an optical device. In some embodiments, the nanoelectronic devices comprise a mechanical sensor. In some embodiments, the nanoelectronic devices comprise a stimulator. In some embodiments, the nanoelectronic devices comprise an actuator.

In certain embodiments, the step of embedding the nanoelectronic devices comprises transferring the nanoelectronic devices onto a two-dimensional sheet of cells and allowing the cells to aggregate, proliferate, and migrate. In some embodiments, allowing the cells to aggregate, proliferate, and migrate compresses the nanoelectronic devices and embeds them within the cells in the tissue.

In some embodiments, the cells are living. In certain embodiments, the cell are in vivo. In some embodiments, the tissue is living. In some embodiments, the tissue is in vivo. In certain embodiments, the tissue is three-dimensional. In some embodiments, the tissue is any tissue with electrical activity. In some embodiments, a tissue is any tissue with a physiological signal that can be detected by an electrical sensor. In some embodiments, a tissue is any tissue that generates or responds to an electrical signal. In certain embodiments, the tissue is brain tissue. In certain embodiments, the tissue is heart tissue. In certain embodiments, the tissue is pancreas tissue. In certain embodiments, the tissue is nervous system tissue. In certain embodiments, the tissue is muscle tissue. In certain embodiments, the tissue is gastrointestinal tract tissue. In certain embodiments, the tissue is eye or ear tissue. In certain embodiments, the tissue is adrenal gland, breast, or salivary gland tissue. In certain embodiments, the tissue is developing tissue. In some embodiments, the tissue is an organoid. In some embodiments, the tissue is human induced pluripotent stem cell-derived. In certain embodiments, the cells are stem cells. In some embodiments, the cells are progenitor cells. The present disclosure also contemplates the use of diseased tissue in any of the systems disclosed herein (e.g., tissue samples obtained from a subject who has been diagnosed with or is otherwise thought to have a disease). In some embodiments, the diseased tissue comprises cardiac tissue (e.g., from a subject with a cardiac disease). In some embodiments, the diseased tissue comprises neurological tissue (e.g., from a subject with a neurological disease). In some embodiments, the diseased tissue comprises skin (e.g., from a subject with a dermatologic disease). In some embodiments, the diseased tissue comprises a tumor sample (e.g., from a subject with cancer).

### Kits

Further disclosed herein are kits. The kits provided may comprise one or more components needed for correlating electrophysiological activity and gene expression in cells in a tissue as described herein. In some embodiments, a kit described herein further includes instructions for using the kit.

In one aspect, kits comprising one or more nanoelectronic devices are provided. In some embodiments, each nanoelectronic device comprises one or more sensors with a unique electronic barcode. In some embodiments, the electronic barcode is a fluorescence electronic barcode. In some embodiments, the electronic barcode is a photodiode barcode or a transistor barcode. In another aspect, the present disclosure provides kits comprising any of the systems disclosed herein.

The kits contemplated by the present disclosure may be used for correlating electrophysiological activity and gene expression in a cell, for preparing a tissue for continuous electrophysiological recording, for discovering disease targets, for drug screening, or for any other suitable use, as one of ordinary skill in the art will readily appreciate.

### Methods for Discovering a Target for Treating a Disease and for Drug Screening

Also disclosed herein are methods for discovering a target for treating a disease, the method comprising correlating electrophysiological activity and gene expression in cells in a tissue by the steps of:
(a) embedding nanoelectronic devices in a first tissue to form a nanoelectronics-tissue hybrid, wherein each nanoelectronic device comprises a unique electronic barcode;
(b) performing continuous electrophysiological recording on the cells;
(c) fixing the nanoelectronics-tissue hybrid;
(d) performing in situ single-cell transcriptome sequencing on the cells;
(e) performing transcriptomic mapping on the cells;
(f) identifying the position of the electronic barcode within the nanoelectronics-tissue hybrid;
(g) performing cell segmentation to correlate the single-cell transcriptome sequencing data with the electrophysiological recording data; and
(h) repeating steps (a)-(g) on a second tissue, wherein the second tissue is engineered as a disease model, and comparing the single-cell transcriptome data and electrophysiological recording data from the first tissue and the second tissue.

In some embodiments, a disease is a neurological disease. In some embodiments, a disease is a cardiovascular disease. In some embodiments, a disease is a muscle disease. In some embodiments, the disease is a dermatologic disease. In some embodiments, the disease is cancer. In some embodiments, a disease is a disease related to any of the tissues discussed herein. In certain embodiments, a disease is any disease related to a tissue with electrical activity.

Also disclosed herein are methods for screening for a drug to treat a disease, the method comprising correlating electrophysiological activity and gene expression in cells in a tissue, wherein the tissue is engineered as a disease model, by the steps of:
(a) embedding nanoelectronic devices in the tissue to form a nanoelectronics-tissue hybrid, wherein each nanoelectronic device comprises a unique electronic barcode;
(b) performing continuous electrophysiological recording on the cells;
(c) fixing the nanoelectronics-tissue hybrid;
(d) performing in situ single-cell transcriptome sequencing on the cells;
(e) performing transcriptomic mapping on the cells;
(f) identifying the position of the electronic barcode within the nanoelectronics-tissue hybrid;
(g) performing cell segmentation to correlate the single-cell transcriptome sequencing data with the electrophysiological recording data; and
(h) repeating steps (a)-(g) in the presence of a drug and comparing the single-cell transcriptome sequencing data and the electrophysiological recording data with the data obtained in the absence of the drug.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Nanoelectronic Devices

Also disclosed herein are nanoelectronic devices comprising one or more sensors, wherein each sensor comprises a unique electronic barcode. In some embodiments, the barcode is a fluorescence electronic barcode. In some embodiments, the electronic barcode is a photodiode barcode. In some embodiments, the electronic barcode is a transistor barcode. In certain embodiments, the barcode comprises a unique binary code. The nanoelectronic devices contemplated herein may comprise varying numbers of sensors.

### EXAMPLES

### Example 1

Human induced pluripotent stem cell-derived cardiomyocytes (hiPSC-CMs) are promising cell sources for cardiac transplantation¹. Building reliable hiPSC-CM tissue resources requires determination of the functional maturation (e.g., electrical, mechanical and gene expression activities) of three-dimensional tissues and organoids over the time course of development, which remains a major challenge. If met, it will provide critical insights into in vitro cardiac development and variations of patient-specific hiPSC-derived tissue samples for patient-specific applications².

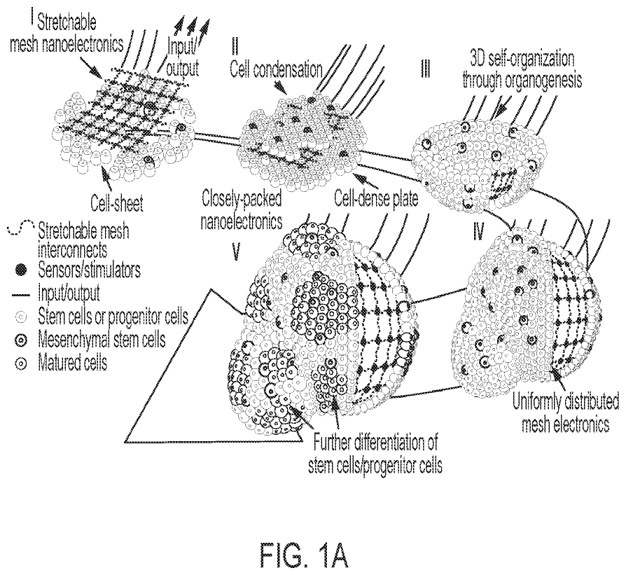

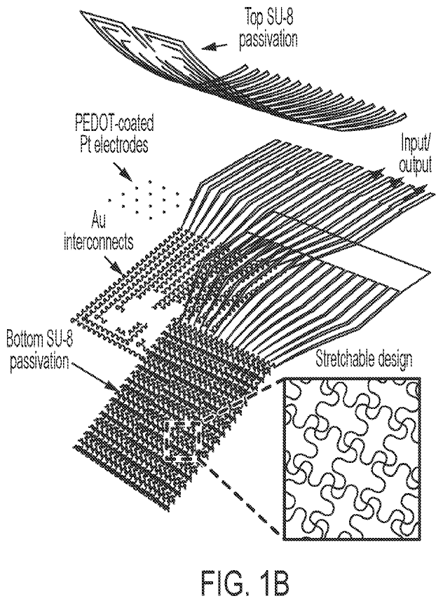

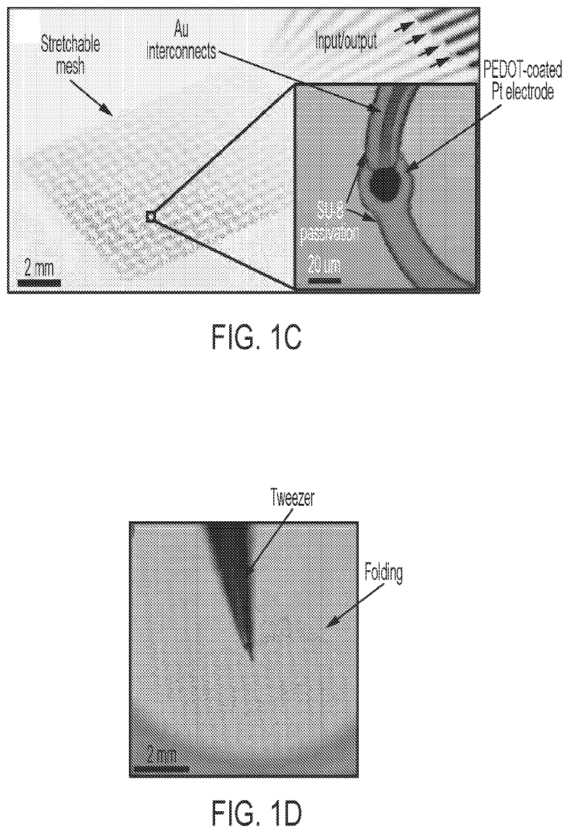

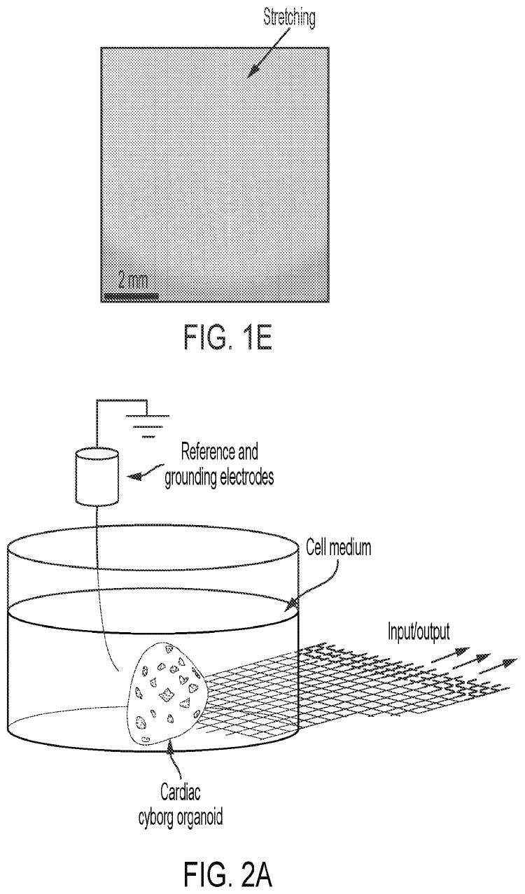

To address this challenge, a cyborg organoid technology was recently developed³ that implanted and distributed stretchable mesh nanoelectronics across hiPSC-derived cardiac organoids through tissue development, enabling continuous three-dimensional (3D) electrophysiological interrogation of developing cardiac organoids (FIG. 1A). The first step consists of transferring and laminating a mesh-like plane of nanoelectronics along with its input/output (I/O) lines onto a 2D sheet of stem cells or progenitor cells (Stage I). Attraction forces between cells during cell aggregation, proliferation, and migration gradually shrink the cell sheet into a cell-dense plate, which simultaneously compresses the nanoelectronics into a closely packed architecture and embeds them within the cells (Stage II). This interwoven cell—nanoelectronics structure then contracts and curls as a result of organogenesis-induced self-folding, first into a bowl geometry (Stage III), and then into a 3D spherical morphology (Stage IV). During this process, the mesh nanoelectronics seamlessly reconfigure with the cell plate due to their soft mechanics, while maintaining uniform spatial distributions throughout the tissue, leading to a fully grown 3D organoid with an embedded sensor/stimulator array in a minimally invasive and globally distributed manner (Stage IV), hence the name “cyborg organoid.” Finally, the stem cells in the as-formed cyborg organoid can further differentiate into targeted types of functional cells, such as cardiomyocytes, while their electrophysiological activities can be chronically monitored using the embedded nanoelectronics (Stage V). Several important design characteristics of the nanoelectronics are underlined, which enable the as-described seamless, tissue-wide integrations (FIG. 1B). First, the mesh design exploits a serpentine layout with an overall filling ratio of less than 11%, leading to a significantly improved in-plane stretchability of up to 30% and out-of-plane compressibility several times smaller than its initial volume due to buckling of the mesh network. This design enables the accommodation of drastic volumetric changes (mostly compressive) during organogenesis^{4,5}. Second, both sets of device dimensions implemented (ribbon width/thickness=20/0.8 μm and 10/2.8 μm) result in minimal device mass (less than 15 μg) and effective bending stiffness of 0.090 nN·m and 1.9 nN·m, respectively, that are essentially imperceptible to the surrounding tissues. FIG. 1C shows the mesh nanoelectronics immediately before release from the fabrication substrate, while the inset shows an individual platinum electrode electrochemically deposited with poly(3,4-ethylenedioxythiophene) (PEDOT) to further lower the interfacial impedance. Folding (up to 180°) and stretching (up to 30% biaxially) the released device in a chamber filled with water for 100 cycles reveals neither is visualizably damaged (FIGS. 1D and 1E).

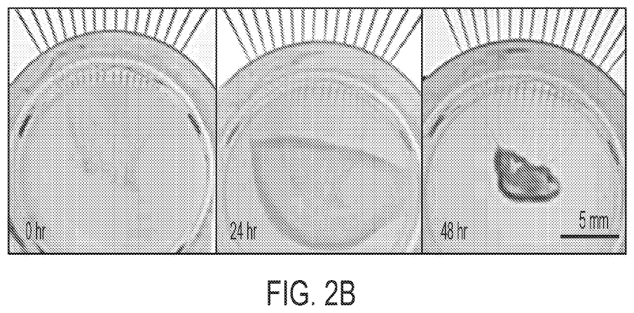

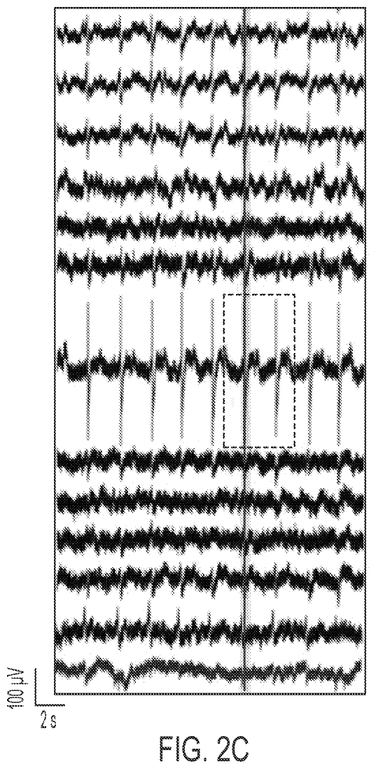

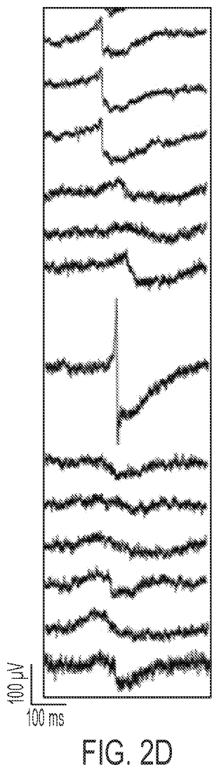

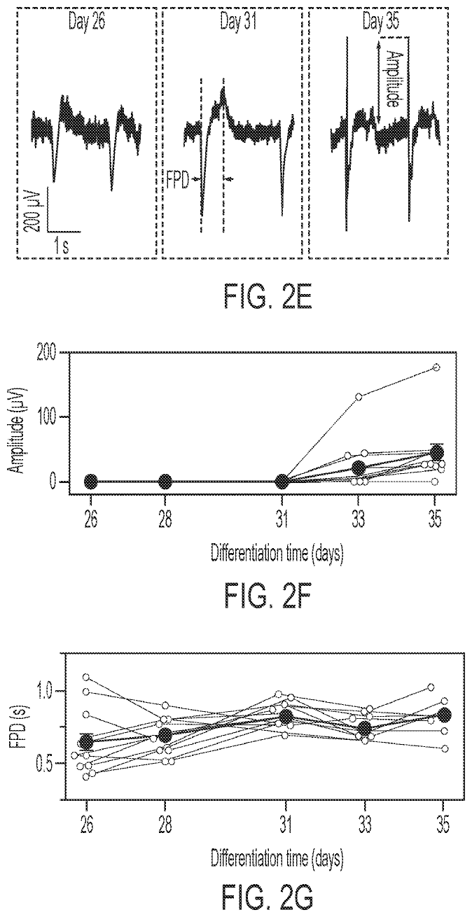

The strong coupling between stem/progenitor cells and the nanoelectronics offers a unique opportunity to map the evolution of cellular electrophysiology throughout organogenesis. As a proof-of-concept, electrophysiological recordings were performed on cardiac cyborg organoids, which typically form in ˜48 h (FIGS. 2A and 2B). FIG. 2C shows the voltage trace of a 14-channel (out of 16 channels) electrophysiological recording of a cardiac cyborg organoid at day 35 of differentiation. The zoom-in plot of a single spike (FIG. 2D) shows nonuniform electrophysiological behaviors of the cells distributed across the organoid, as well as a clear time latency revealing tissue-wide propagation of local field potentials (LFP). Chronic tracing of LFP at millisecond temporal resolution during organogenesis (FIG. 2E) reveals changes in the spike dynamics from an initially slow waveform through the emergence of repolarization to fast depolarization⁶. The averaged amplitude of the fast component associated with depolarization remains undetectable until day 31 of differentiation and then increases monotonically (FIG. 2F). The field potential duration (FPD) is found to remain relatively steady between 0.7 and 0.8 s (FIG. 2G) with a slight increase from day 26 to 35.

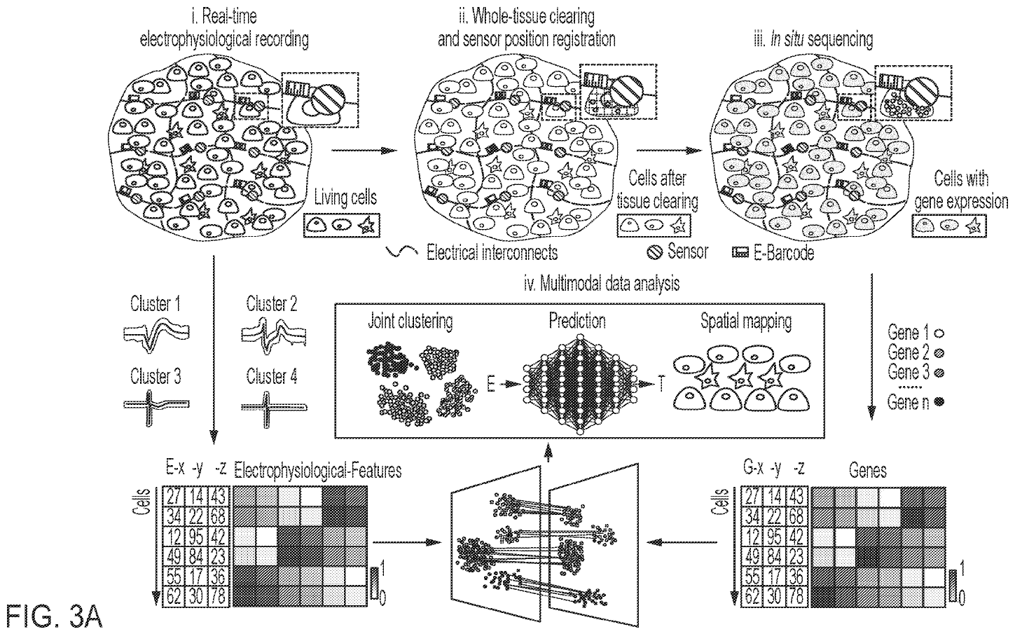

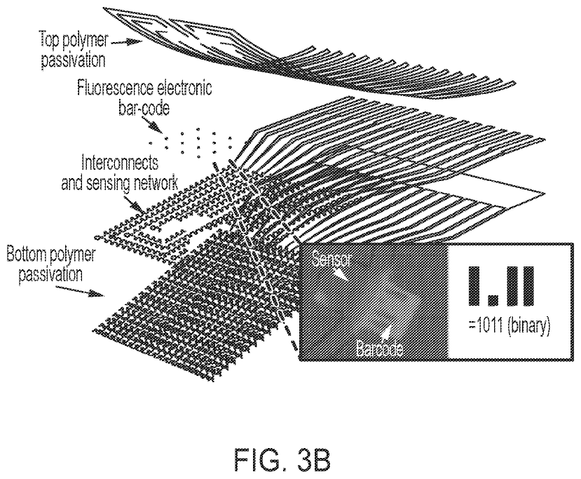

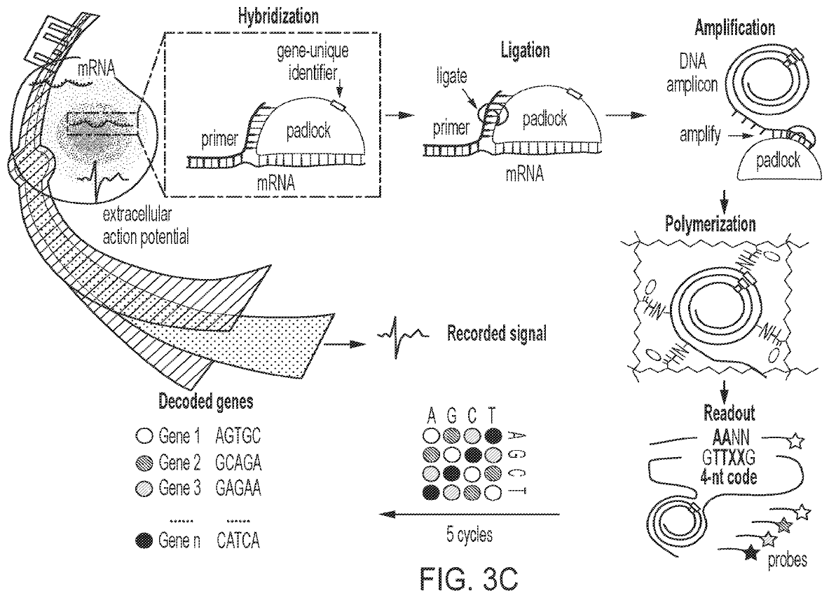

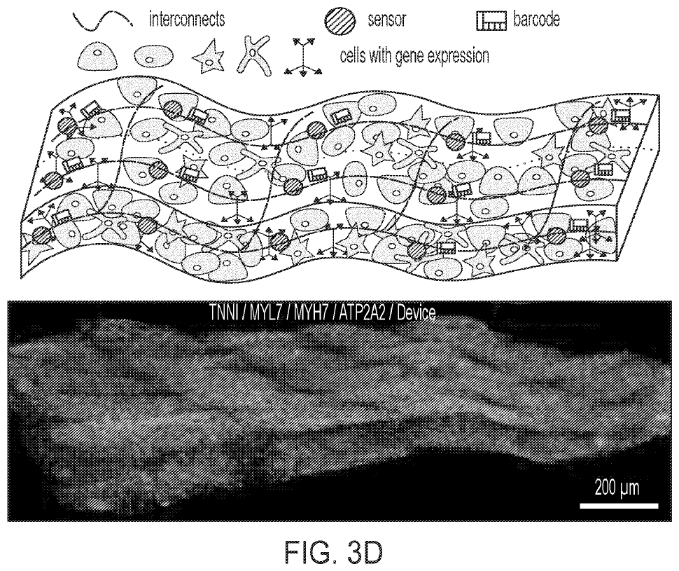

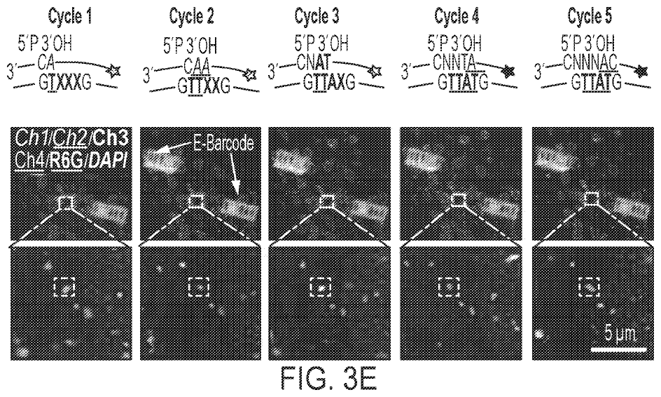

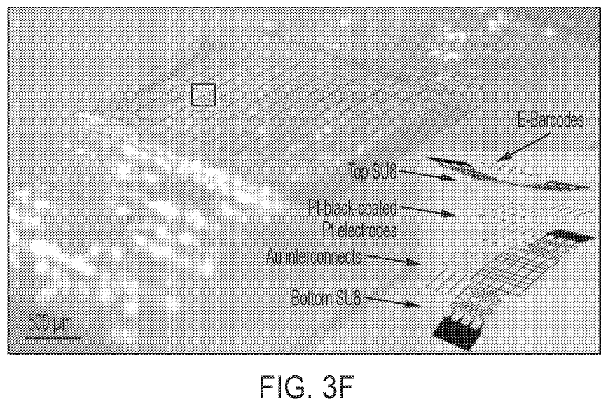

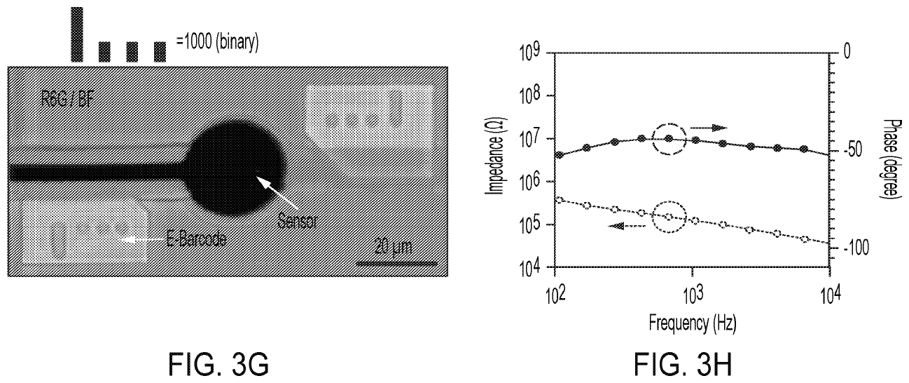

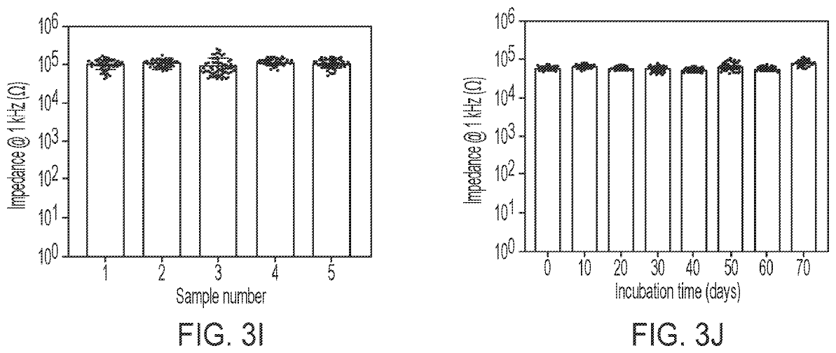

Cyborg organoid technology has offered the opportunity to study the evolution of electrophysiological patterns during organogenesis, revealing the heterogeneity of electrophysiological behaviors of hiPSC-CM over the time course of development. To fully understand the developmental process and heterogeneity in the electrophysiological behaviors, tools are needed that are capable of tracing both electrophysiological behavior and molecular phenotyping (e.g., gene expression) from the same cells over time, which are still not available. To bridge this gap, an in situ single-cell RNA sequencing (scRNA-seq) platform (STARmap)⁸ is further combined with this cyborg organoid technology to simultaneously profile the electrophysiological and transcriptional states of hiPSC-CMs in 3D developing organoids at cellular resolution, which was termed “in situ electrode sequencing”. Specifically, STARmap is a powerful tool to spatially resolve gene expression in tissues at subcellular spatial resolution through confocal fluorescence imaging. By introducing the photolithographically defined fluorescence electronic barcodes to the stretchable electronics, the simultaneous measurement of tissue electrophysiology and gene expression has been successfully demonstrated at single-cell resolution in a high throughput and spatially resolved manner. In situ electrode sequencing consists of the following three key steps (FIG. 3A): (i) The soft nanoelectronics with fluorescence barcoded sensors are embedded in the 3D intact tissue for single-cell electrophysiological recording as described in cyborg organoid technology (FIGS. 3A and B). (ii) After electrophysiological recording, the whole cyborg tissue is fixed, embedded in hydrogel, and cleared for in situ RNA sequencing. DNA amplicons with a pre-designed gene-specific identifier sequence are constructed in situ by probe hybridization, enzymatic amplification, and immobilized in the tissue cleared hydrogel network (FIG. 3C). (iii) A pre-designed gene-specific identifier sequence in a DNA amplicon is read out by multiple rounds of fluorescent imaging (FIG. 3E). Each sensor unit in the nanoelectronic device is uniquely paired with a binary fluorescence barcode, thus enabling registration of the sensor position with cell position. After the in situ electrode sequencing, the fluorescence spatially imaged sensor and cell positions are used to integrate the electrophysiological and gene expression information for the cells.

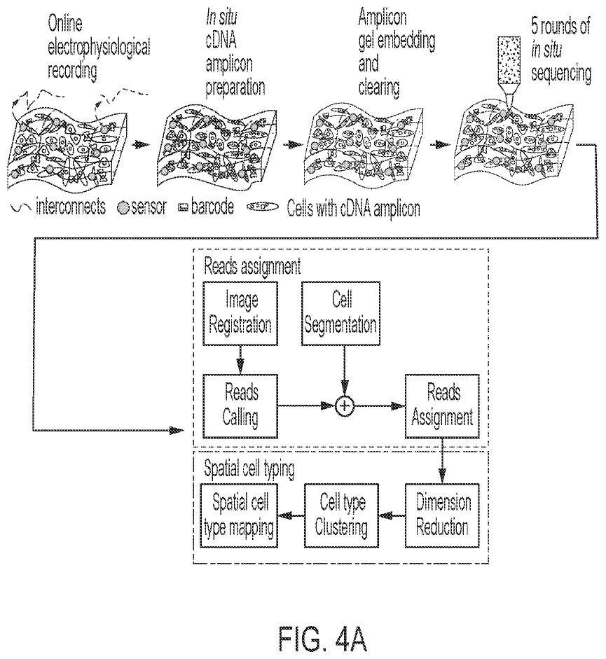

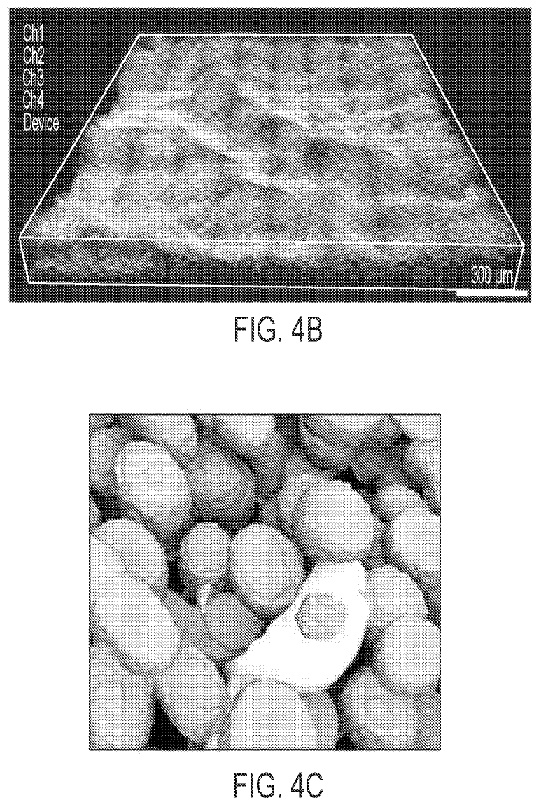

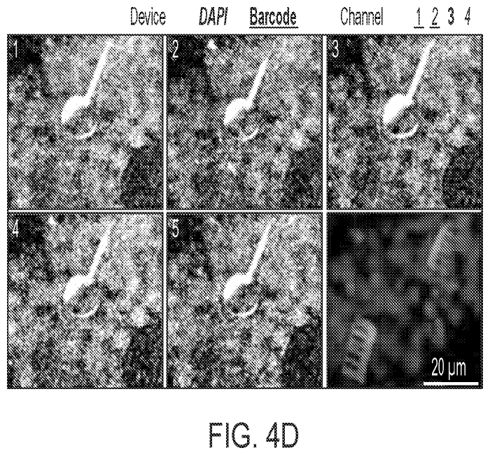

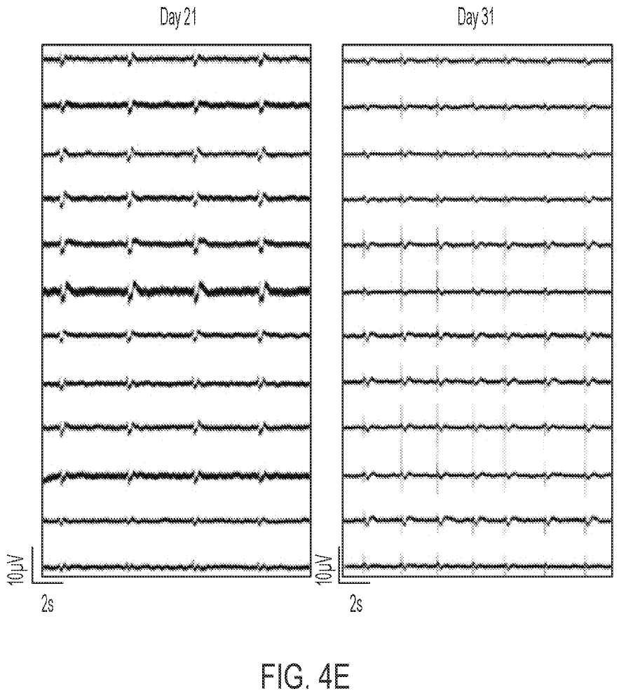

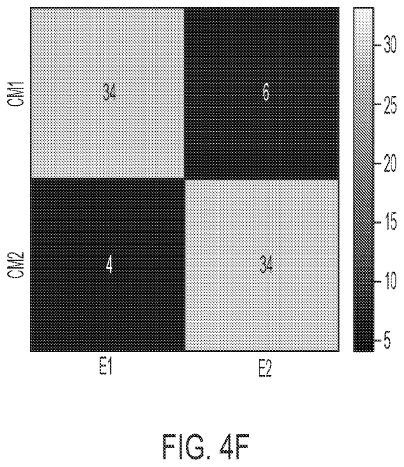

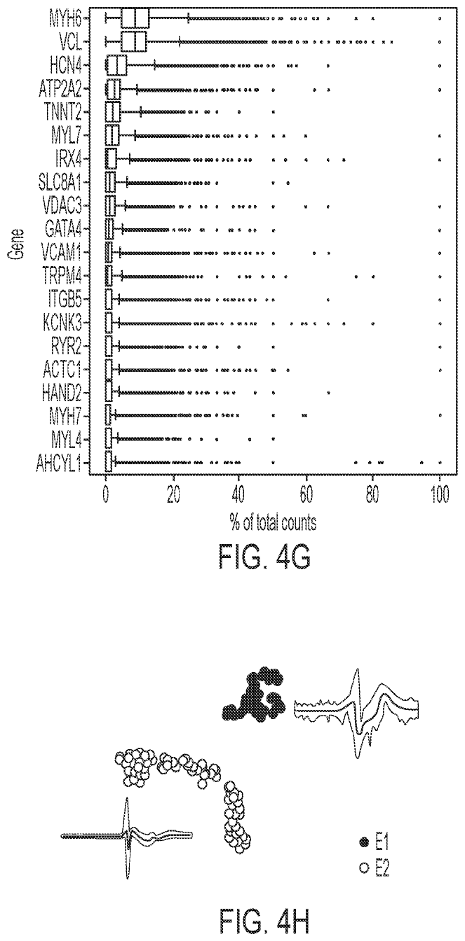

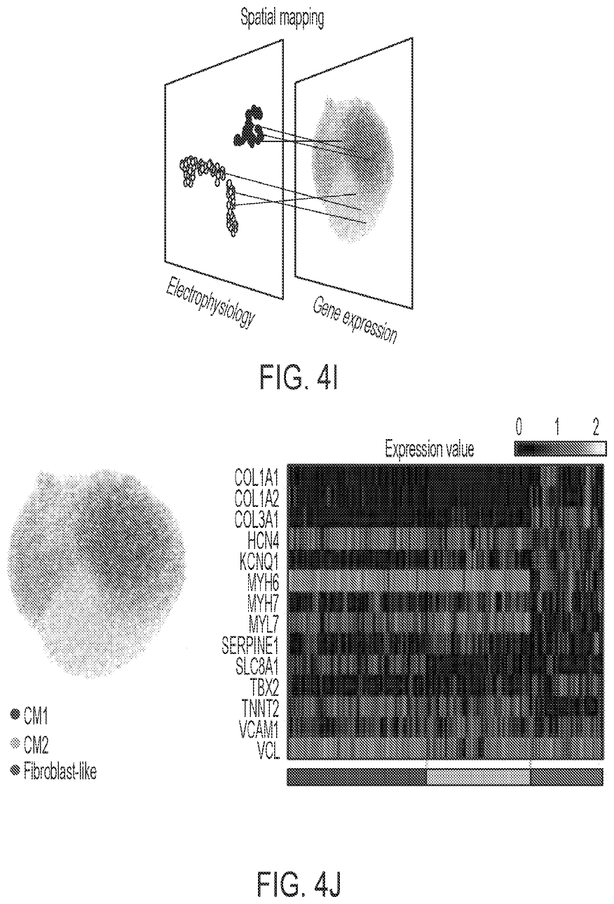

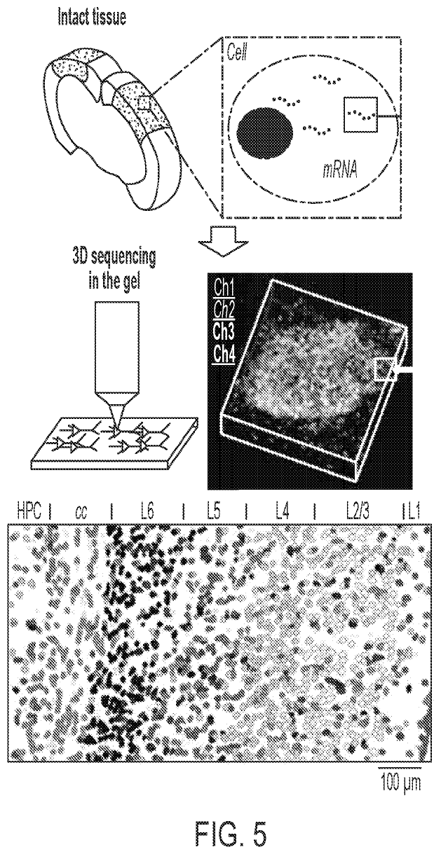

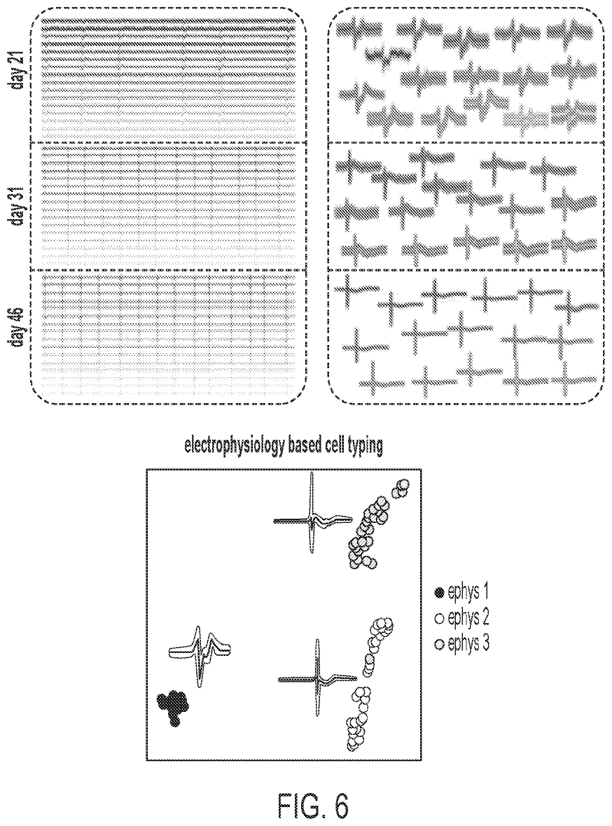

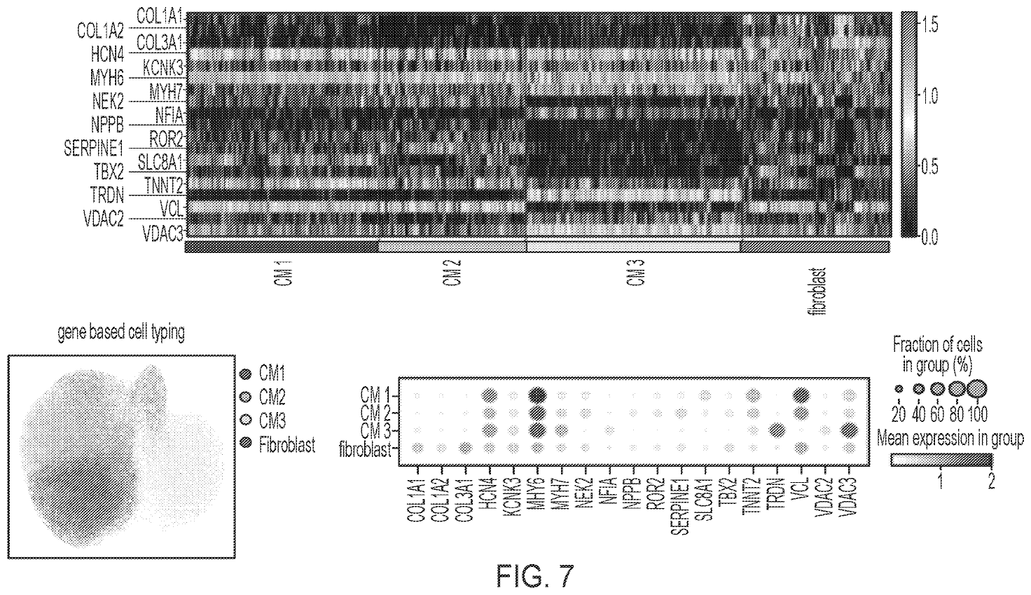

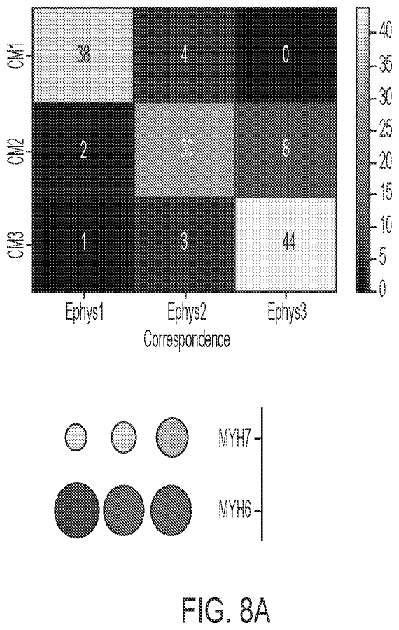

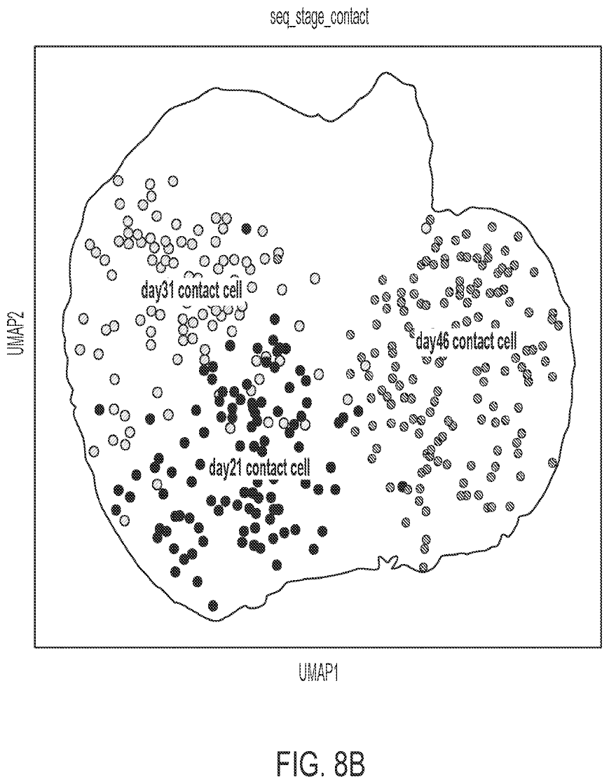

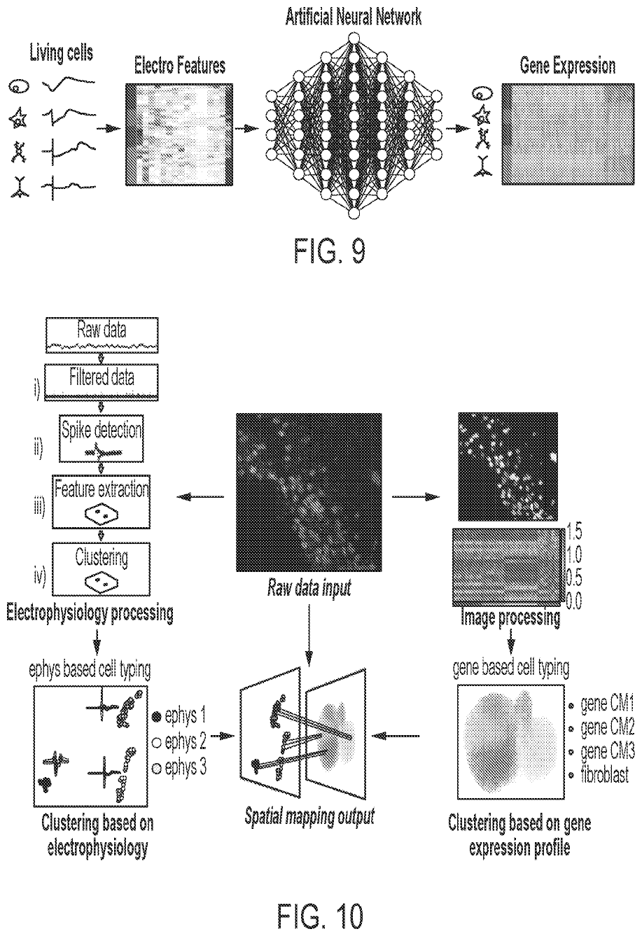

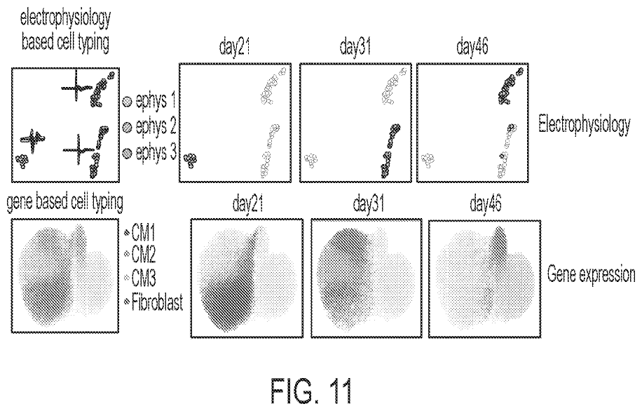

As a validation of the method, the electrophysiological recordings were first performed on cyborg cardiac tissue at day 21 and day 31 of differentiation (FIG. 4A). The representative voltage traces (FIG. 4E) of 12 channels out of 64 channels of electrophysiological recording reveal distinct electrophysiological features between day 21 and day 31 cyborg cardiac tissue. Principle component analysis (PCA) was performed to extract the 15 most variant principle components as feature vectors for each electrophysiological channel and use Gaussian mixture model (GMM) to unsupervised cluster these feature vectors. After projecting the principle components into two-dimensional uniform manifold approximation plot (UMAP)⁹, two distinct clusters (named as E1 and E2) can be seen, representing electrophysiological states of cells from day 21 and 31,respectively.

Next, day 21 and day 31 cyborg cardiac tissues were fixed for in situ scRNA-seq (FIGS. 4A and 4D). 3D reconstructed fluorescence imaging after the first round of sequencing showed the interwoven 3D nanoelectronics/cellular structure across the entire cyborg cardiac tissue (FIG. 4B). 200 genes from single-cell RNA sequencing of the hiPSC-CM dataset are selected for sequencing (mainly including differentially expressed genes across the in vitro cardiac developmental process)¹⁰. Five rounds of sequencing and one round of imaging to register the device barcodes with cell positions were performed (FIG. 4D). A customized computational pipeline was built to decode gene identity and assign gene reads to each cell nuclei (FIG. 4A): (i) fluorescence images of cDNA amplicon dots in five sequencing rounds were registered with a phase correlation algorithm followed by local distortion registration¹¹; (ii) DNA amplicons/dots were identified in each individual color channel using the centroid of 3D regional maximum from the first sequencing round. Then the dominant color for the identified dot in each following round was determined. Color sequence for each dot was compared to the codebook, and the gene identity for each dot was decoded; and (iii) cell segmentation was performed by using STARdist neural network¹². Each RNA read was then assigned to the cell. Using the pipeline, the top 20 enriched genes (FIG. 4G) were analyzed, confirming that most genes are related to cardiac structural and regulation transcription factors (e.g., Tnnt2, Myh6, Gata4, Hand2). Unsupervised cell type clustering was then performed, and the gene expression profile was projected into a UMAP plot. Cells in the hiPSC-derived cardiac tissues can be generally separated into two major groups (FIG. 4J): fibroblast like cells and cardiomyocytes (CM1 and CM2). Fibroblast like cells are mainly responsible for collagen production, thus not contributing to the tissue electrical behaviors¹³. Cardiomyocytes highly expressed Tnnt2, Myh6, Myh7 and My17 genes (FIG. 4J), which are sarcomere related genes responsible for muscle contraction regulation¹⁰. The cardiomyocytes can be further separated into two subpopulations (CM1 and CM2) with different gene expression pattern.

Finally, fluorescence imaging is used to integrate the fluorescence barcodes for sensor positions and DAPI signals for cell positions to integrate the electrophysiological states and transcriptional states of cells (FIGS. 4C and 4I). FIG. 4F shows the correspondence between cardiomyocytes defined by electrophysiology features (E1 and E2) and gene expression (CM1 and CM2). The majority of E1 cells (34 out of 38 E1 cells that passed electrophysiology quality control) matched to CM1 cells, while the majority of E2 cells (out of 40 E2 cells) matched to CM2 cells. Comparing to CM1 cells, CM2 cells show higher structural maturation with increased gene expression in sarcomere related genes such as Myh7 and My17 (FIG. 4J). From the integrated data, it can be concluded that the increased sarcomere gene expression could be potentially correlated with the change from slow waveform in E1 to fast depolarization waveform in E2 (FIG. 4H).

In summary, the first human cardiac cyborg organoids have been created via organogenetic 2D-to-3D tissue reconfiguration, tracing and mapping the evolution of electrophysiological patterns during organogenesis. The in situ electrode sequencing technology was further developed, which integrates electrophysiology measurement and gene expression measurement at single cell resolution, revealing the correlation between the changes of electrophysiological and transcriptional states of iPSC-derived CMs over development. The in situ electrode sequencing can be applied to reveal cell type diversity during complex developmental process by integrating electrophysiological and transcriptomic data for common cell type reference. In addition, in situ electrode sequencing is scalable for integrating a larger number of sensors as well as increasing the number of genes.

The in situ electrode sequencing platform will enable exploring the causal genetic foundation of cellular functional activity (e.g., electrical, mechanical) in a high throughput manner. The methods discussed herein will make contributions to determining the dynamic processes of tissue development and functional and genetic alterations of tissue in disease. In addition, in situ electrode sequencing will serve as a platform for spatially resolved, high-fidelity, joint profiling tool for many other types of tissues and organoids.

### Example 2

Simultaneously charting single-cell gene expression and electrophysiology in intact three-dimensional (3D) tissues across time and space is crucial to understanding the gene-to-function relationship in fields ranging from developmental biology to cardiology and neuroscience¹⁴⁻¹⁸. Such multimodal methods require stable and continuous recording of single-cell electrical activity with high spatiotemporal resolution across 3D tissue, multiplexed profiling of a large number of genes in electrically recorded cells, and cross-modal computational analysis¹⁹.

Large-scale single-cell electrical recording²⁰⁻²² and high-throughput single-cell sequencing²³⁻²⁵ have enabled system-level investigation of single-cell electrophysiology and gene expression, respectively. However, existing multimodal methods either lack high spatiotemporal resolution across the 3D tissue or cannot simultaneously measure tissue-wide electrical activities in a long-term stable manner. For example, combining calcium imaging with RNA hybridization²⁶ can reveal the correlation of calcium activity and molecularly defined cell types. It, however, can only record the cell activity at second temporal resolution and profile a limited number of genes. Patch-seq^{15,16,27} quantifies cell activity at millisecond temporal resolution and profiles the whole transcriptomes of the recorded cells but assays cells one at a time and requires membrane disruption during the electrical measurement, which is a challenge to tissue-wide and long-term stable electrical activity mapping.

Recent developments in thin-film flexible bioelectronics have enabled soft “tissue-like” electronics, capable of seamlessly integrating with tissue networks for long-term stable, millisecond temporal resolution single-cell electrical mapping^{3,28-31}. Meanwhile, current imaging-based in situ sequencing methods⁸ can achieve end-point spatial analysis of thousands of genes at subcellular resolution across intact 3D tissues.

Here, soft bioelectronics were integrated with in situ sequencing as one method termed “in situ electro-seq” to enable a scalable and simultaneous profiling of single-cell electrophysiology and gene expression in intact 3D tissues. In situ electro-seq was applied to 3D human induced pluripotent stem cell-derived cardiomyocyte (hiPSC-CM) patches, and precisely registered the CM gene expression with electrophysiology at single-cell level, enabling joint cell clustering and pseudotime analysis. Such multimodal integration substantially improved the reconstruction of developmental trajectory and the dissection of cell types from spatially heterogeneous tissues. Through cross-modal analysis, in situ electro-seq identified the gene-to-electrophysiology relationship over the time course of cardiac maturation. Such a relationship was further leveraged to train a coupled autoencoder and predict single-cell expression profile evolution using long-term electrical measurement from the same cardiac patch or 3D millimeter scale cardiac organoids. As exemplified by cardiac tissue maturation, in situ electro-seq is broadly applicable to create spatiotemporal multimodal maps and predictive models in electrogenic organs, allowing discovery of cell types and gene programs responsible for electrophysiological function and dysfunction.

### In Situ Electro-Seq Platform

Mesh electronics were applied for in situ electro-seq to seamlessly integrate with tissue for continuous recording and to prevent potential cell-to-sensor dislocation from tissue development, sample preparation, or multiple cycles of in situ sequencing (FIG. 3A). To do so, a tissue-electronics-hydrogel double network, capable of co-deformation during volume change was formed. Meanwhile, to precisely identify the electrically recorded cells in 3D tissues, photolithography was used to pattern the thin-film microscale polymeric structures with unique fluorescent electronic barcodes (E-barcodes), paired with each individual sensor to label their recording channel during fluorescence imaging cycles of in situ sequencing.

In situ electro-seq consists of the following four key steps (FIG. 3A): (i) the mesh electronics with E-barcoded sensors are embedded in tissues for continuous single-cell electrical recording; (ii) the entire tissue-electronics hybrid is fixed, embedded in hydrogels, and cleared for in situ sequencing; (iii) gene identities and E-barcodes are simultaneously read out by multiple cycles of fluorescence imaging, integrating electrical recording with gene expression profiling at single-cell resolution; and (iv) the multimodal data are analyzed using joint clustering and cross-modal visualization, correlation, and prediction to illustrate the spatiotemporal gene-to-function relationship.

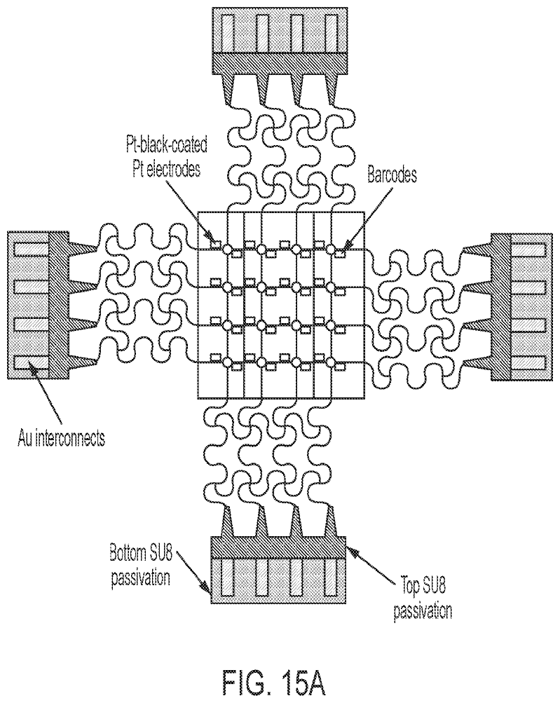

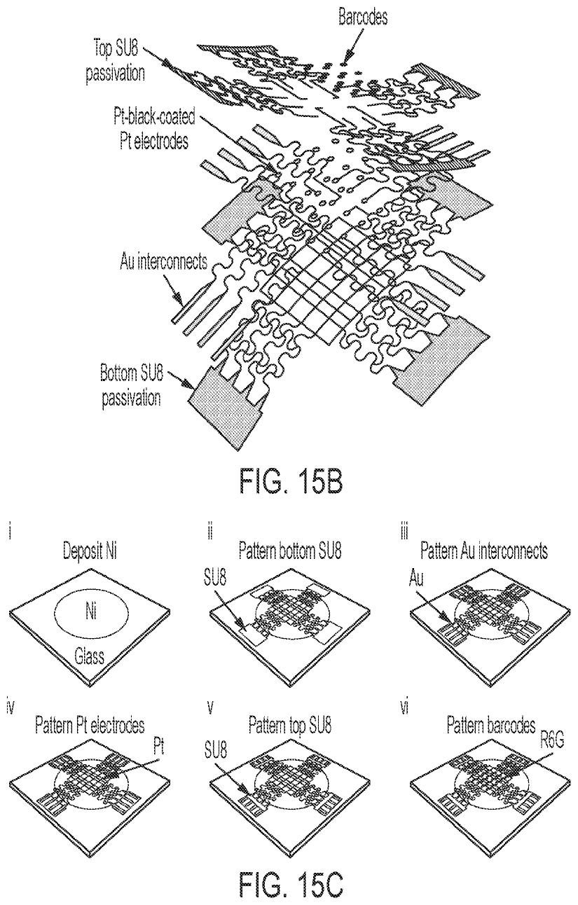

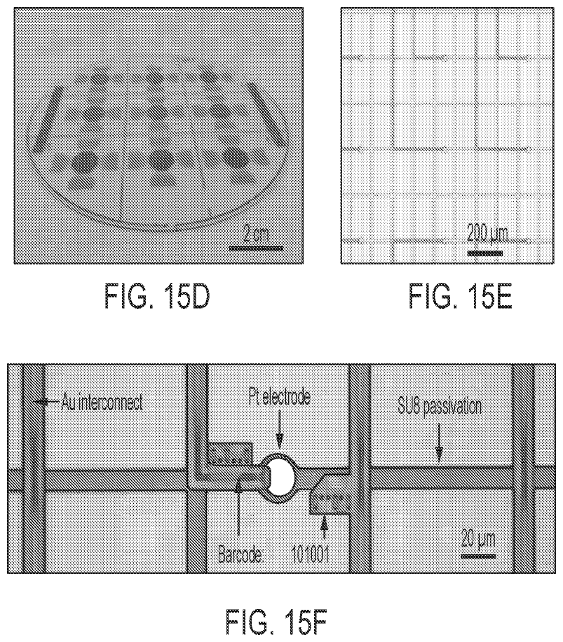

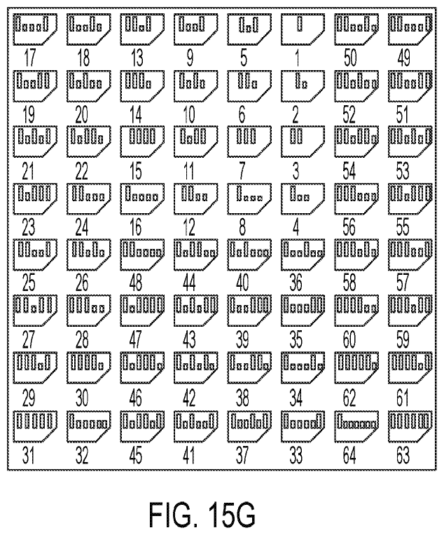

In situ electro-seq was applied to a human induced pluripotent stem cell-derived cardiomyocyte (hiPSC-CM) patch, simultaneously mapping its electrophysiology and gene expression. Representative mesh electronics with 64 electrodes with a 25-μm diameter (FIG. 3F and FIGS. 15A-15E), which approaches the typical size of an individual cell³², enable single-cell electrophysiological recordings. A pair of center-symmetric fluorescent E-barcodes with unique binary codes were patterned with each electrode as center (FIG. 3G and FIGS. 15F-15G). Characterization of electrode impedances (FIG. 3H) showed stable performance across different samples (FIG. 3I) and over >2 months incubation in the physiological solution (FIG. 3J) for long-term electrical recording.

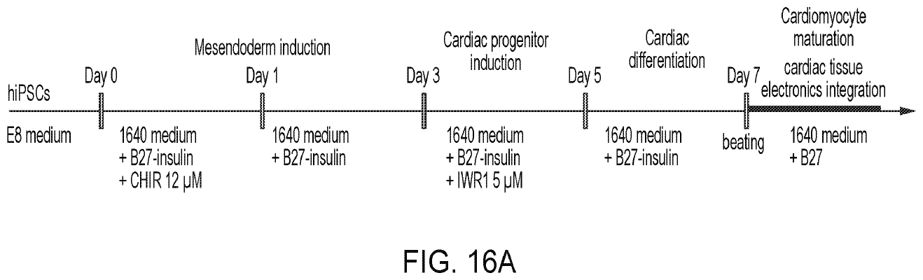

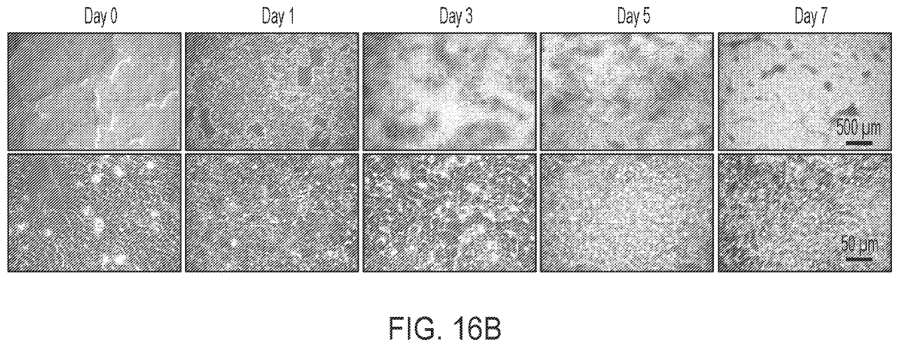

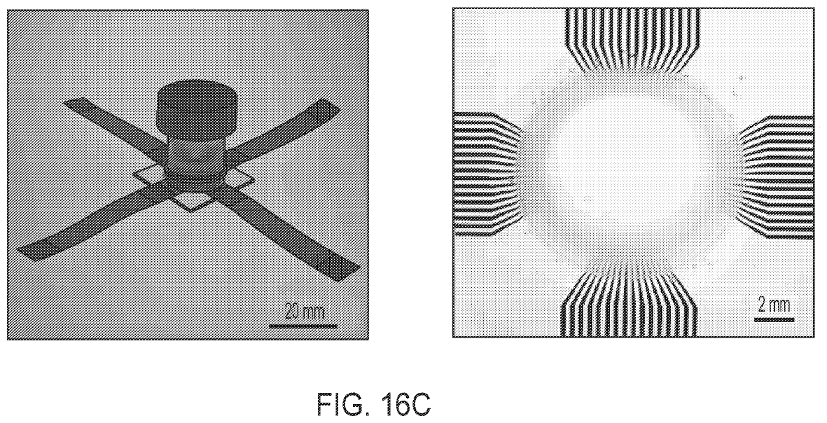

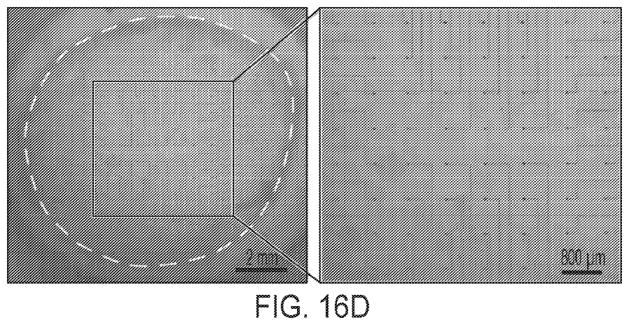

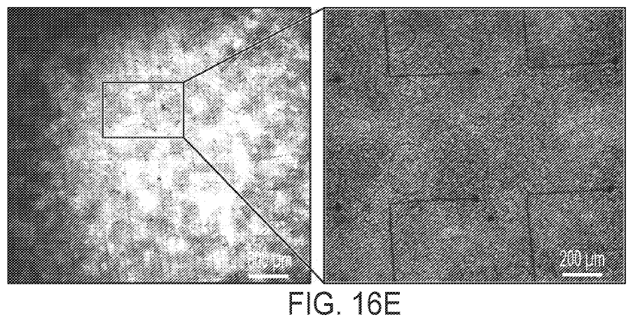

hiPSC-CMs were cultured with mesh electronics on a Matrigel layer to form a 3D cardiac patch as described in previously reported methods^{3, 31} (FIGS. 16A-16E, see Methods). After co-culturing cells with mesh electronics, the tissue-electronics hybrid was fixed, and STARmap in situ sequencing protocols were applied to in situ profile a targeted set of cardiac genes, including the 201 most differentially expressed genes selected during cardiac maturation from published single-cell RNA sequencing (scRNA-seq) data^{10, 33}. RNA-derived DNA amplicons with pre-designed gene-specific identifiers were synthesized in situ by probe hybridization, enzymatic amplification, and immobilization in the cleared tissue-electronics-hydrogel network (FIG. 3C). Then, the gene-specific identifier was decoded through five sequencing cycles. Notably, the microscale distances among E-barcodes, cells, and amplicons remained during the multiple cycle imaging (FIG. 3E).

### In Situ Electro-Seq Enabled Correlation of Electrophysiological and Transcriptional Data

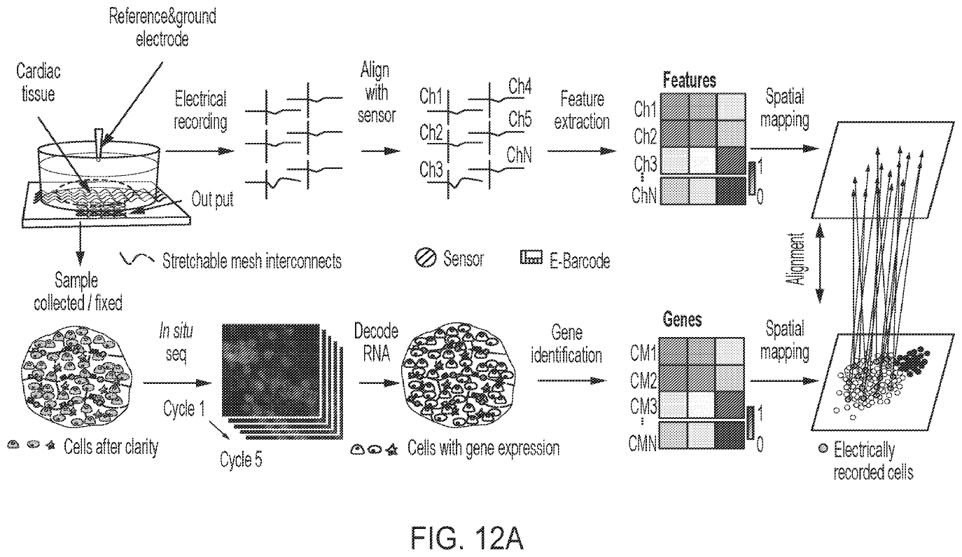

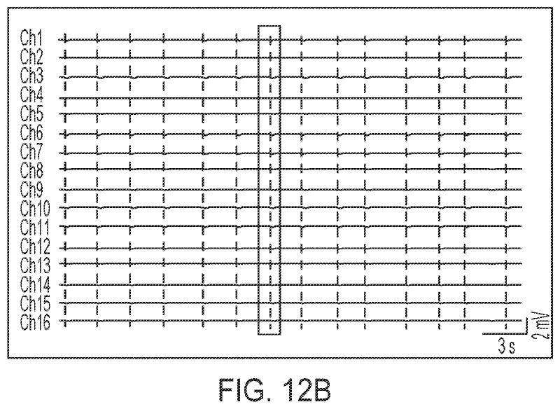

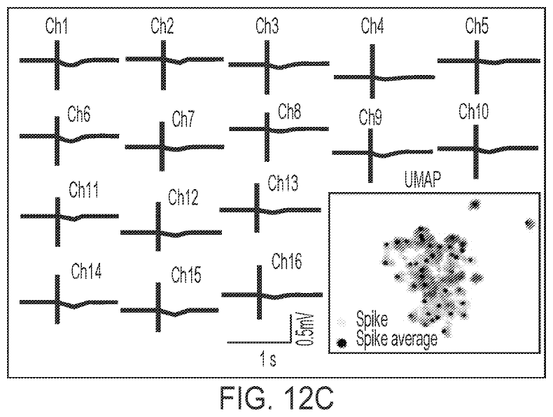

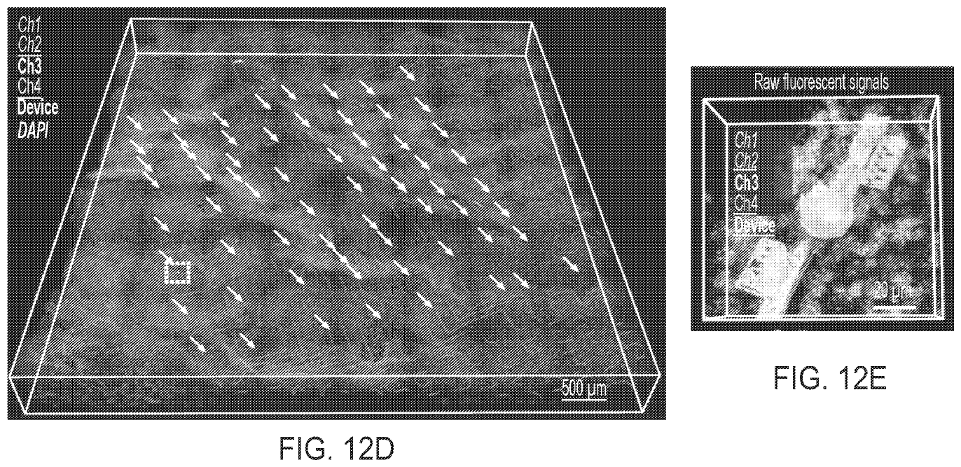

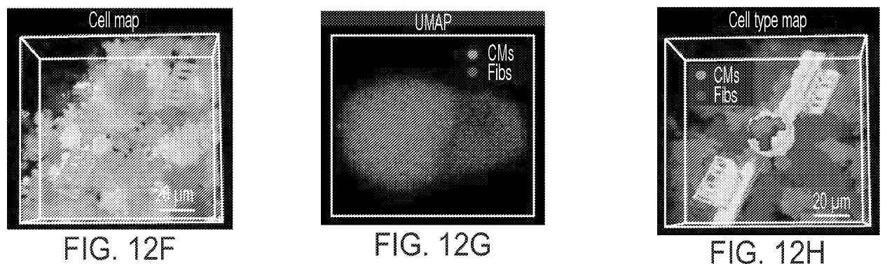

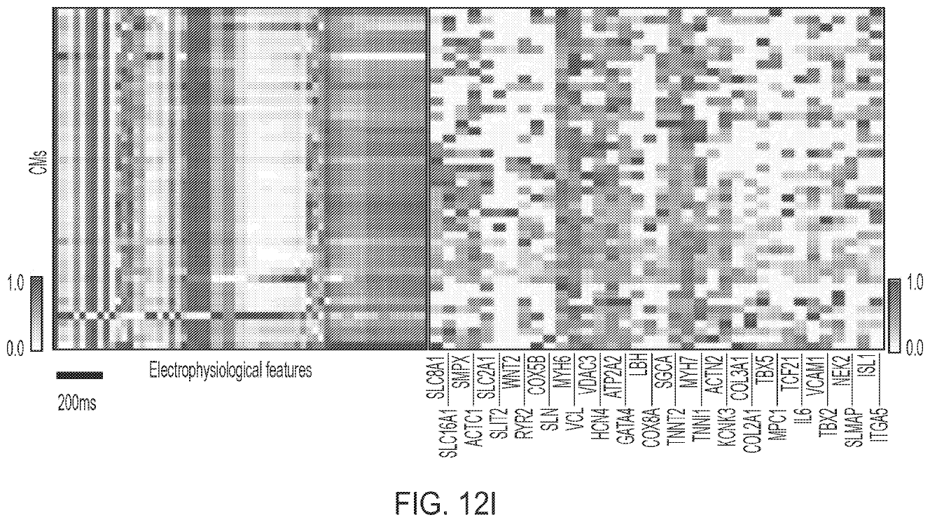

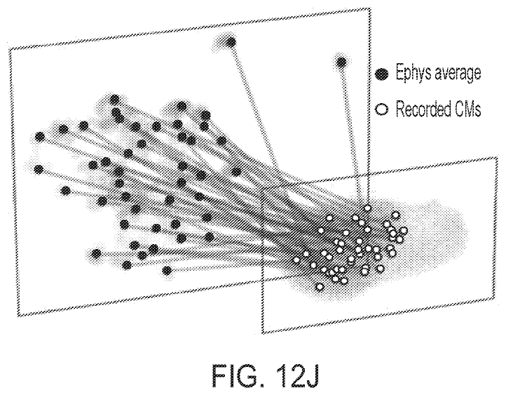

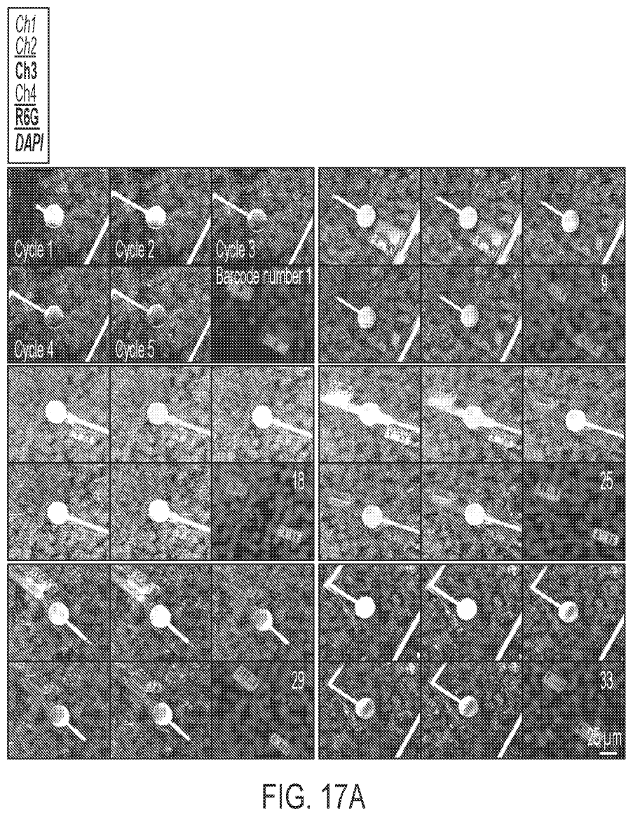

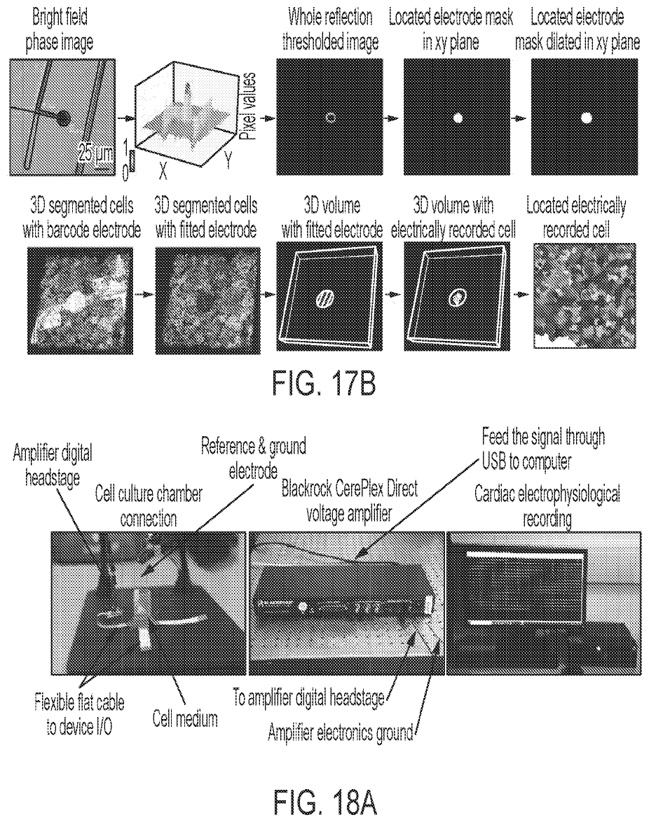

In situ electro-seq was first tested on the hiPSC-CM cardiac patch at Day 46 of differentiation (FIG. 12A). 64-channel recording was applied, and single-spike action potential waveforms were identified, pre-processed, averaged, and down-sampled to extract 62 features. FIGS. 12B and 12C show representative 16-channel voltage traces and single-spiked waveforms, respectively. The uniform manifold approximation and projection (UMAP)²⁷ visualization (FIG. 12C, inset) of extracted features from 64 channels shows the heterogeneity of hiPSC-CM electrophysiology. In situ sequencing was applied immediately after electrical recording (FIG. 12D, FIG. 17A). After 3D cell segmentation (ClusterMap³⁴, FIGS. 12E-12F), cell clustering was performed using 201 genes from 32,429 cells across all the imaged positions. Leiden clustering³⁵ showed two major cell types (FIG. 12G), CMs and fibroblasts (Fibs), which were spatially mapped back to E-barcoded electrodes (FIG. 12H). A computational pipeline was built to automatically identify CMs that formed in direct contact with electrodes as electrically recorded cells (FIG. 12H and FIG. 17B). Next, identification of E-barcodes registered the electrophysiological features with gene expression of the electrically recorded cells. Heatmap (FIG. 12I) and joint UMAP (FIG. 12J) visualization showed the integrative z-scored electrophysiological features and 24 top differentially expressed CM-related genes, and their multimodal distributions at single-cell level, respectively. Together, these data demonstrate that in situ electro-seq can identify the gene-to-electrophysiology relationship at single-cell resolution.

### In Situ Electro-Seq of Cardiac Maturation

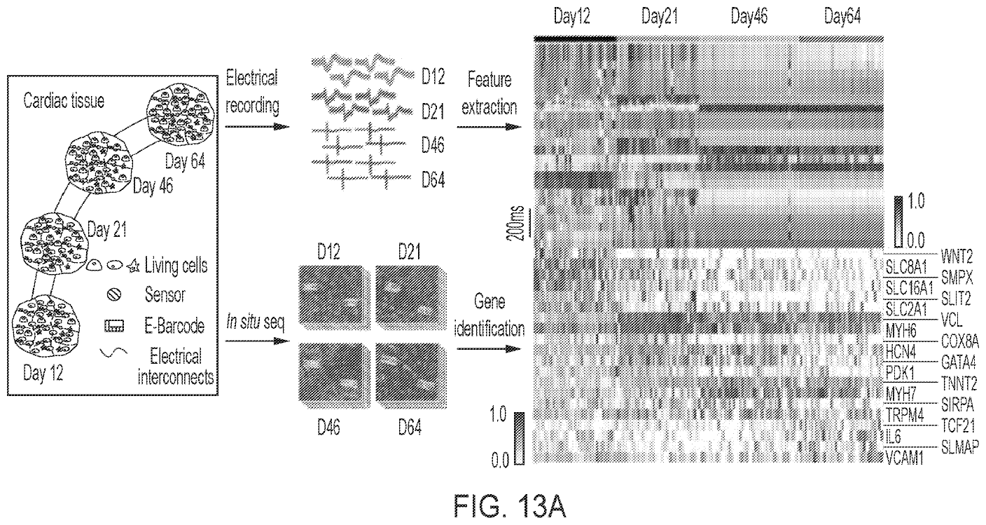

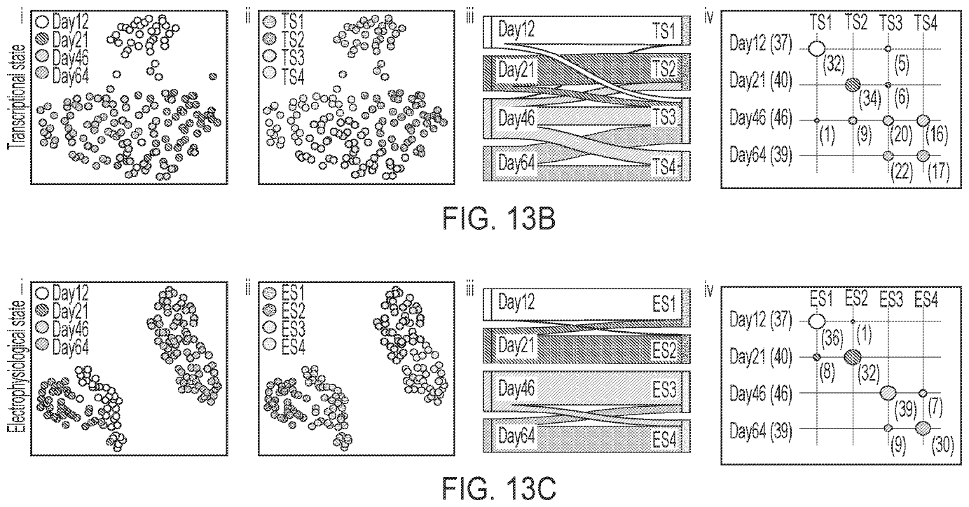

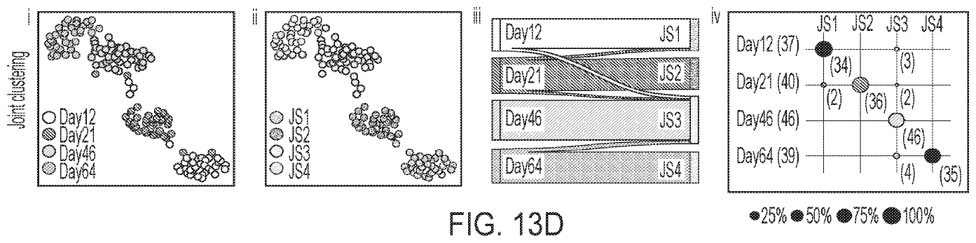

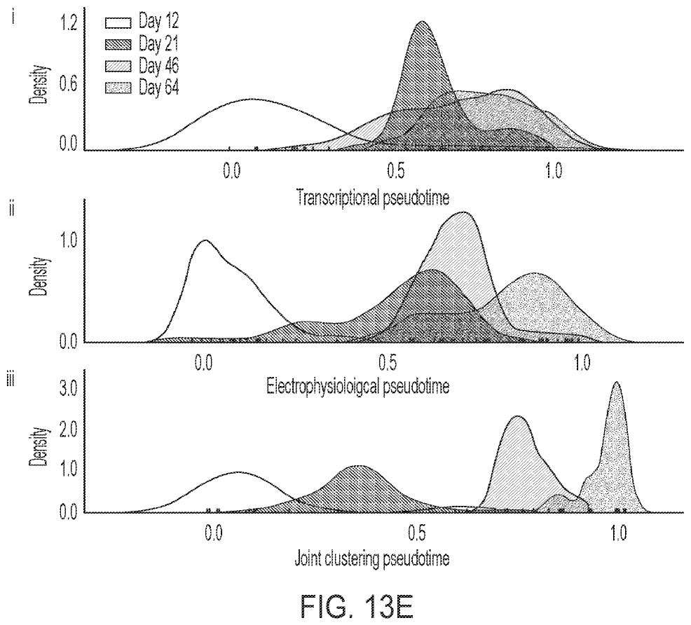

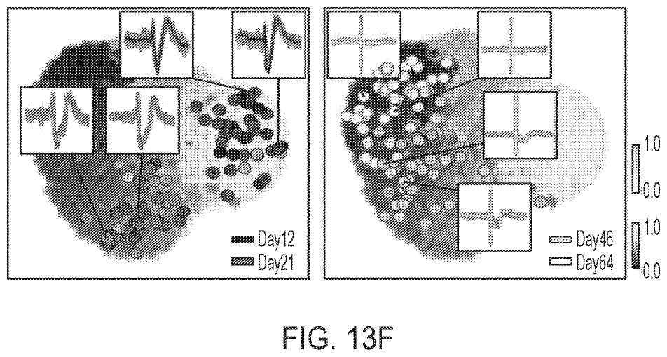

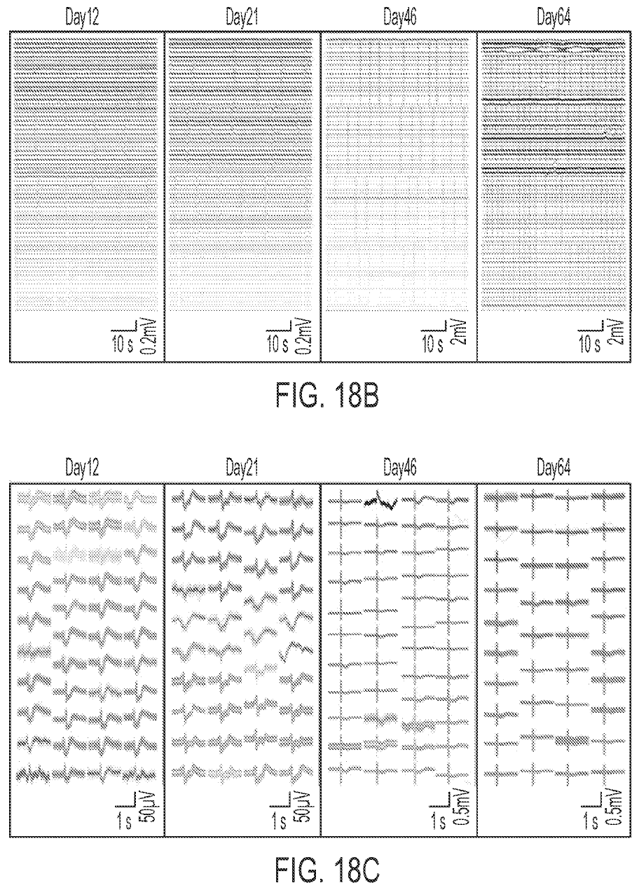

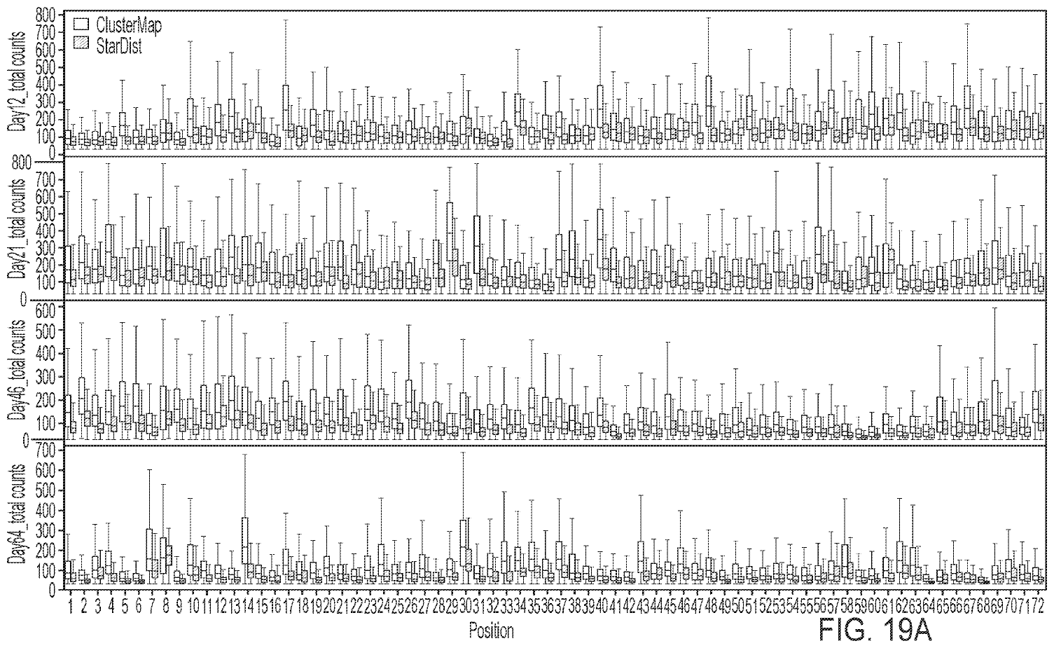

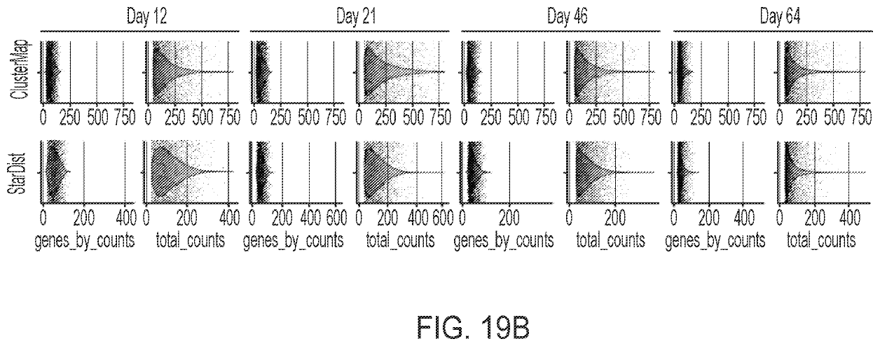

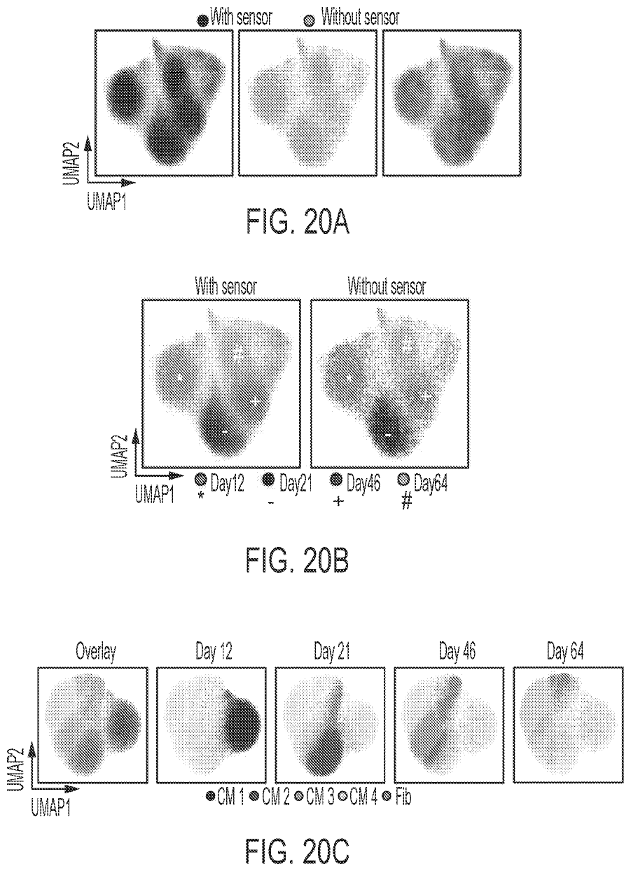

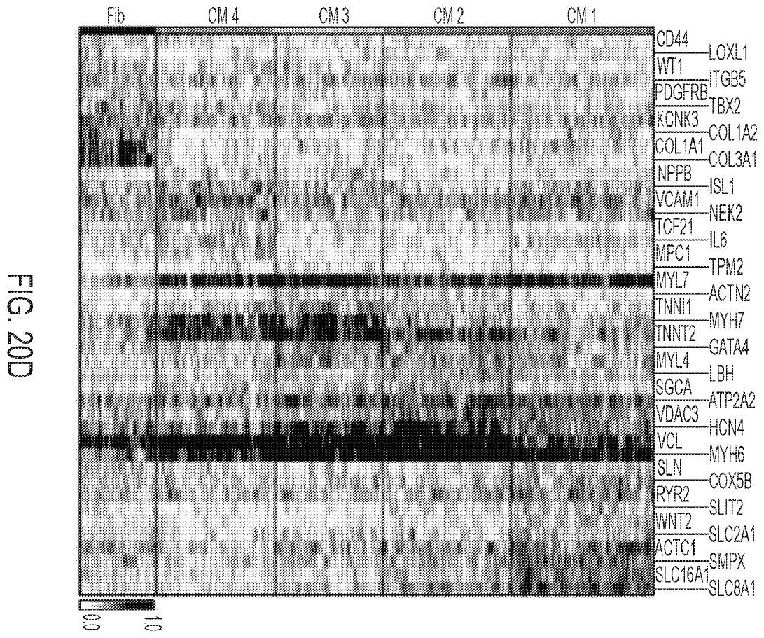

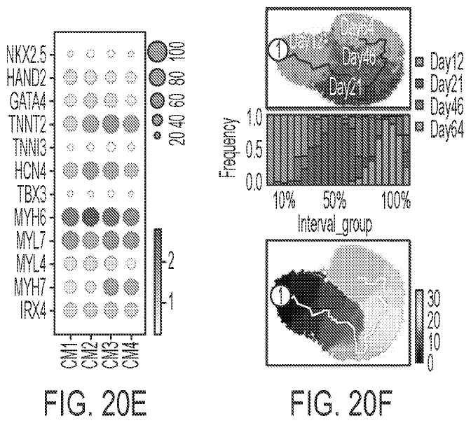

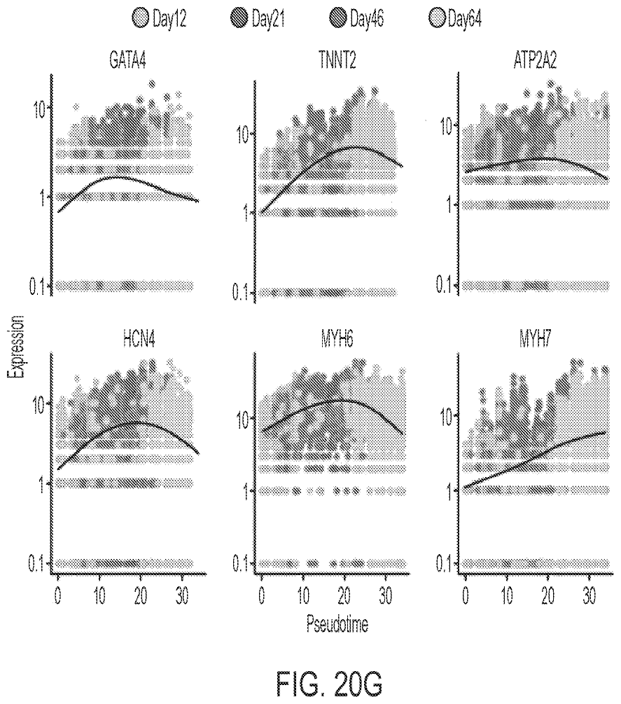

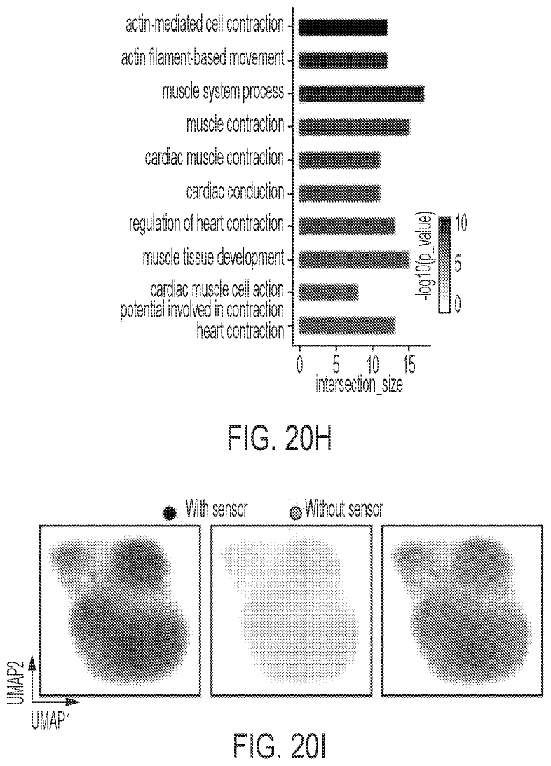

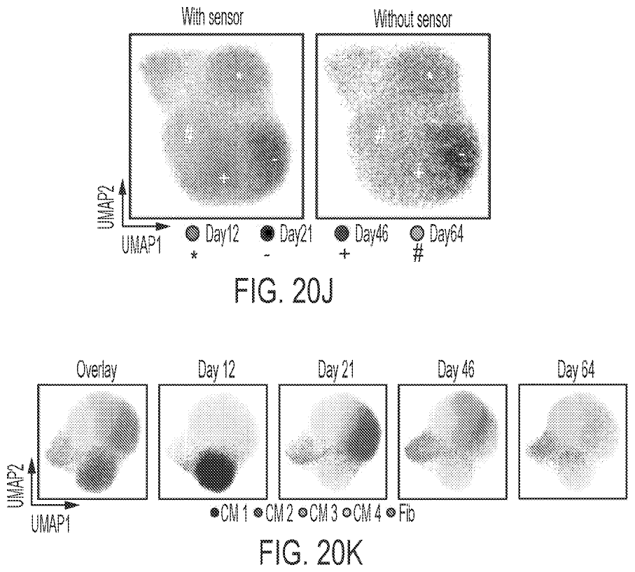

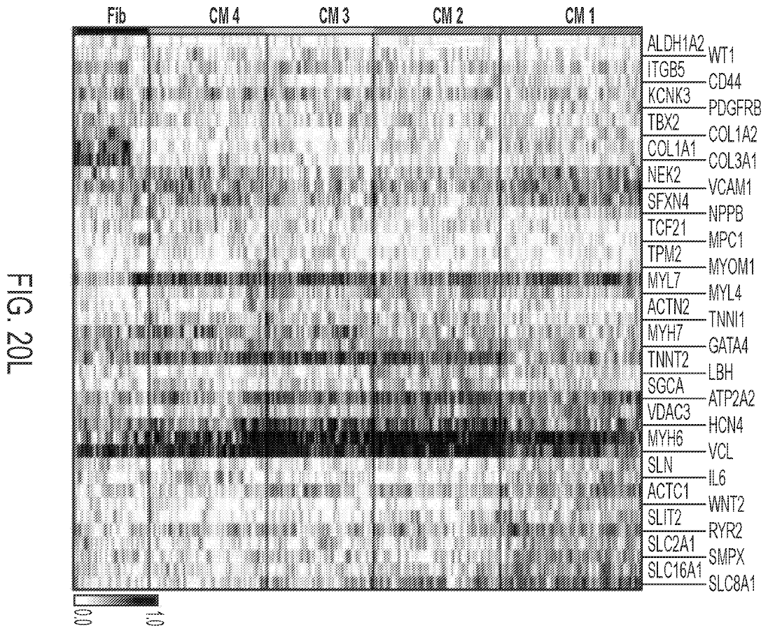

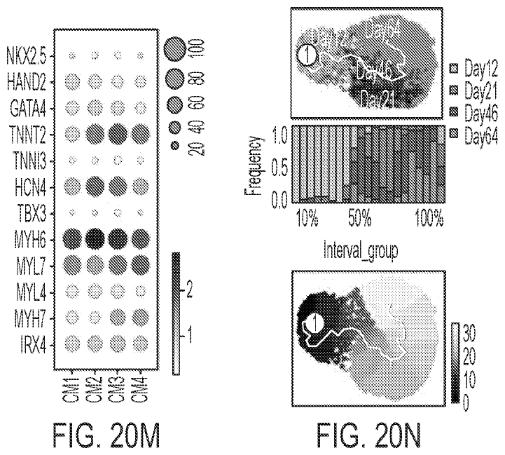

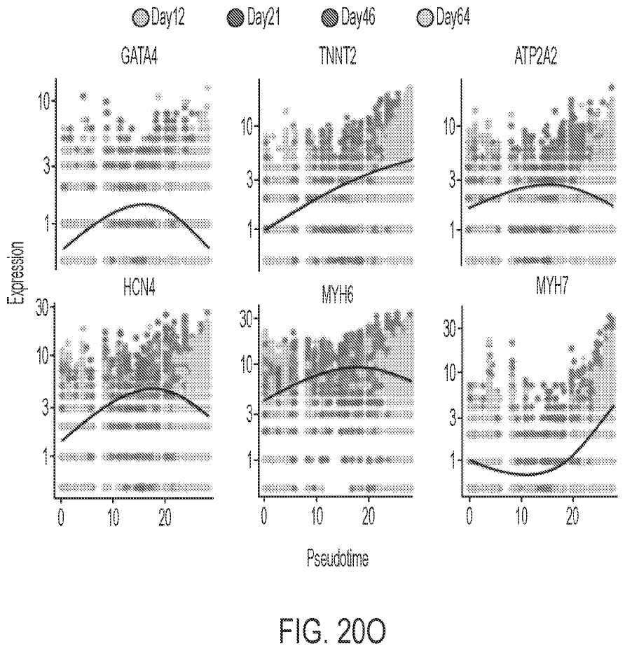

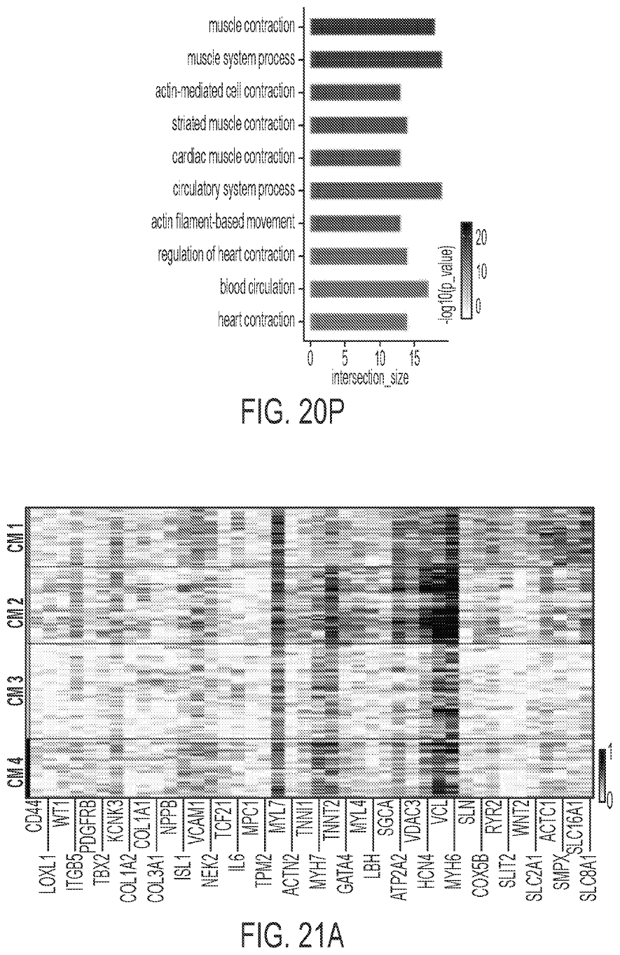

In situ electro-seq was used to trace the maturation of the hiPSC-CM patch (FIG. 13A). The cell electrophysiological signals from Day 12, Day 21, Day 46, and Day 64 of differentiation (FIGS. 18A-18C) showed distinct features at these four stages (FIG. 13A). ClusterMap was chosen to segment cells and compared with StarDist³⁶. ClusterMap can identify higher and stabler RNA counts-per-cell across samples robustly at different differentiation days (FIGS. 19A-19B) and show similar cell-typing results as StarDist (FIGS. 20A-20P). Comparing UMAP visualization of cell types across four differentiation stages showed the same embedding distributions between electronics-embedded and control samples, which suggests negligible effects of mesh electronics on cardiac maturation and performance of in situ sequencing (FIGS. 20A-20B). Unsupervised Leiden clustering analysis was performed on all of the in situ sequenced cells (130,162 cells) across 4 stages, which identified 5 cell clusters (FIG. 20C). Based on the expression levels of the marker genes, four subpopulations can be attributed to CMs and one to fibroblasts. Compared with previous reports³⁷⁻³⁸, the expression level of marker genes (e.g., HCN4, MYH6, MYH7, MYL7, MYL4, etc.) in these four types of CMs indicates the transition of cardiac states from nodal-like through atrial-like CMs to ventricular-like CMs (FIGS. 20D-20E). This can be further confirmed by the gene expression trajectory and pseudotime through the decreasing expression of nodal marker gene (HCN4) and atrial marker gene (MYH6) at the later stage and increasing expression of the ventricular maker gene (MYH7) (FIGS. 20F-20G). Gene ontology (GO) analysis of the top differentially expressed genes revealed enrichment of genes involved in cardiac muscle contraction, conduction, and development (FIG. 20H). Collectively, these data demonstrate that the in situ electro-seq protocol can identify the evolution of electrophysiological and transcriptional profiles over the time course of hiPSC-CM maturation.

### In Situ Electro-Seq Enabled Multimodal Joint Clustering

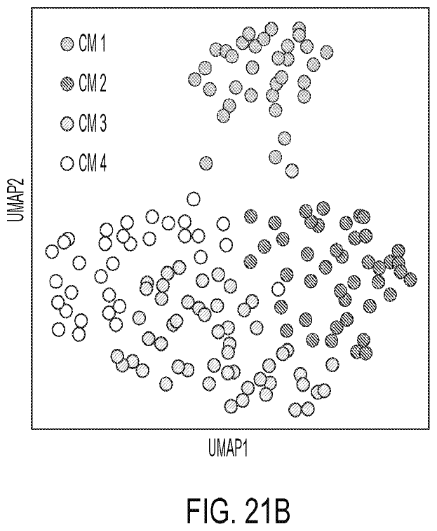

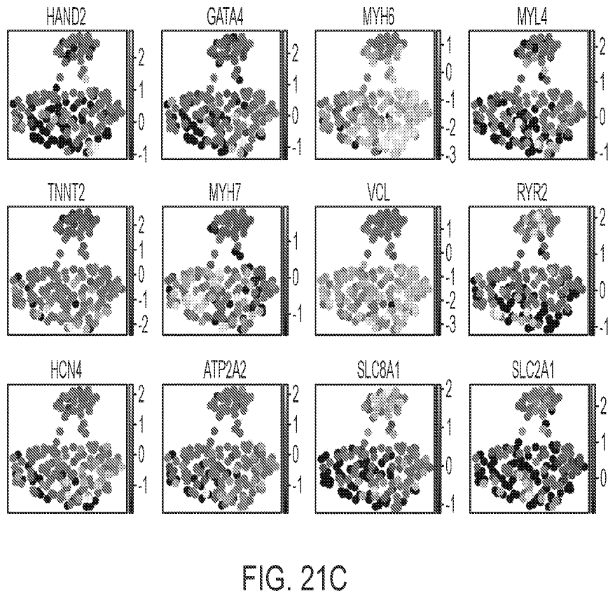

While gene expression clustering can separate CMs into four transcriptional states (t-states), roughly corresponding to samples collected at the four differentiation days, it was observed that the t-states of CMs at Day 46 and 64 of differentiation are less separable (FIG. 20C). The separability of t-states of 162 electrically recorded CMs is worse (FIG. 13B, FIGS. 21A-21C), especially at Day 21, Day 46, and Day 64 of differentiation, which agrees with previously reported scRNA-seq clustering of hiPSC-CMs t-states during maturation³⁸. Using electrophysiological features to cluster the electrically recorded cells, two major electrophysiological states (e-states) can be separated for CMs, one for Day 12 and 21, and the other for Day 46 and 64, respectively. However, the separability within the two major groups is low (FIG. 13C). Previous patch-seq results suggest that integration of gene expression with electrophysiology can improve the classification of cell types³⁹. Here, the weight nearest neighbor (WNN)⁴⁰ algorithm from Seurat v4 was used to integrate the single-cell electrophysiological and gene expression data from in situ electro-seq for joint representation. Using the joint features, CMs can be clustered into four joint states (j-states) that well represent the different differentiation days (FIG. 13D). In addition, Monocle3⁴¹, an unsupervised method, was applied to calculate the pseudotime distributions of t-states, e-states, and j-states. The results also show that the integrated gene expression and electrophysiology data provide a better separation of pseudotime distributions for cells at different differentiation stages (FIG. 13E). Highlighting the 162 electrically recorded CM with their j-state pseudotimes in the UMAP visualization of total 112,892 CMs t-states (FIG. 13F) shows that, with as few as 162 cells, the j-states clearly recapitulated the continuous maturation trajectory of 112,892 CMs (FIG. 20F).

### In Situ Electro-Seq Enabled Cross-Modal Correlation, Prediction and Mapping

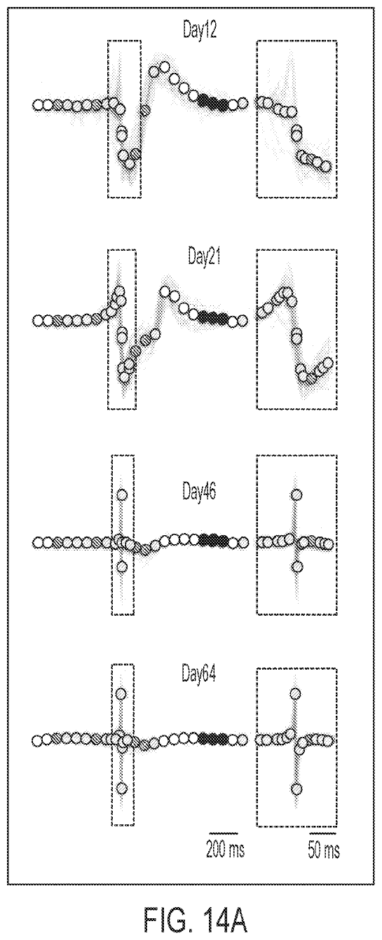

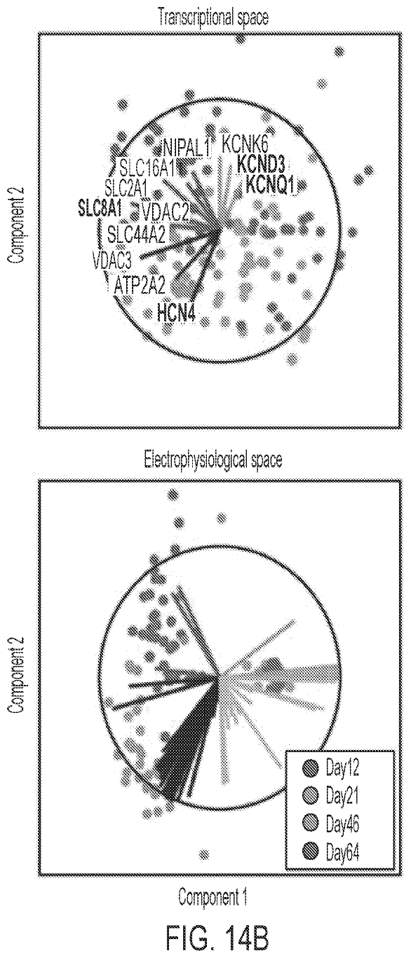

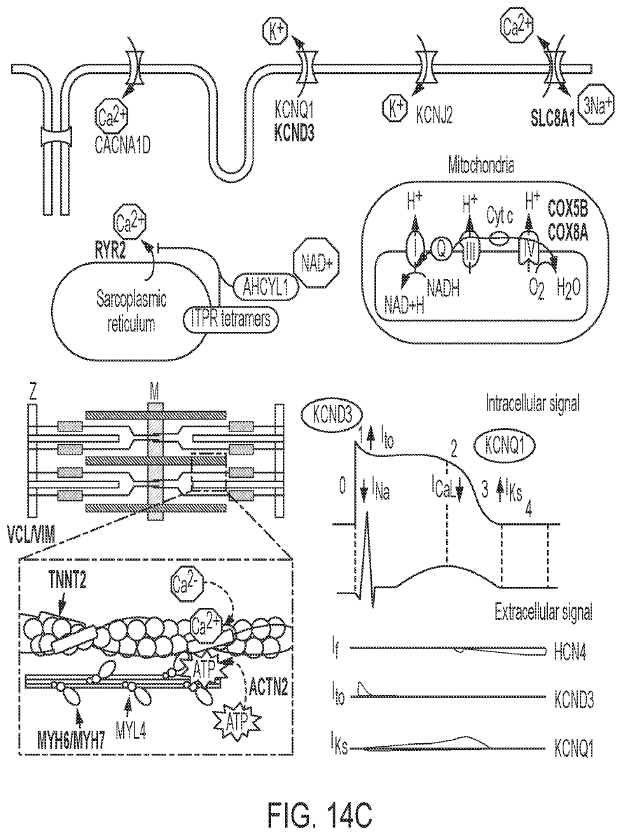

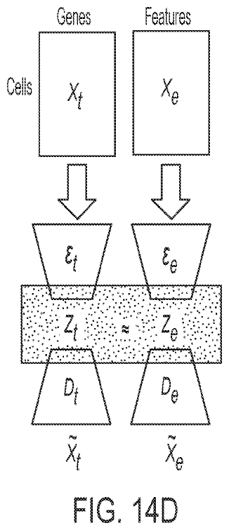

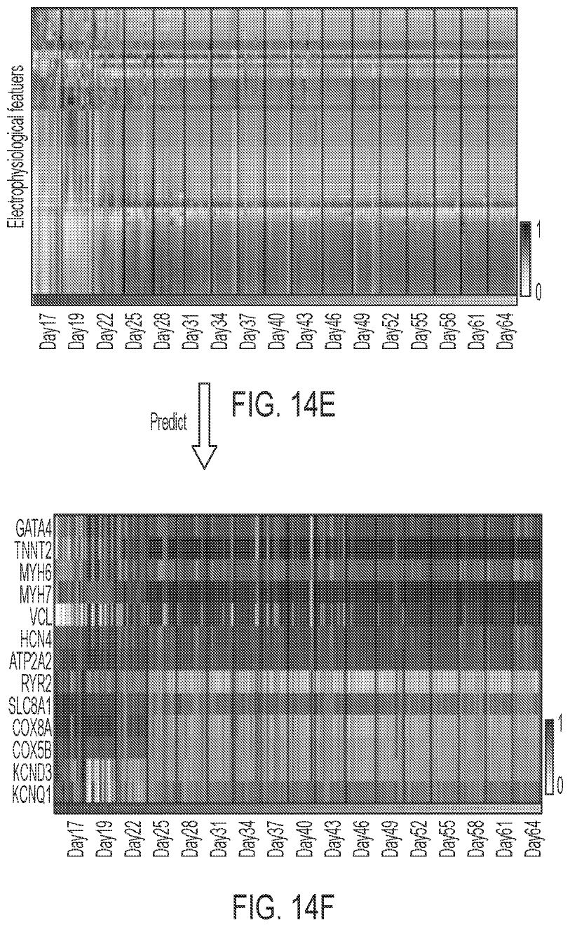

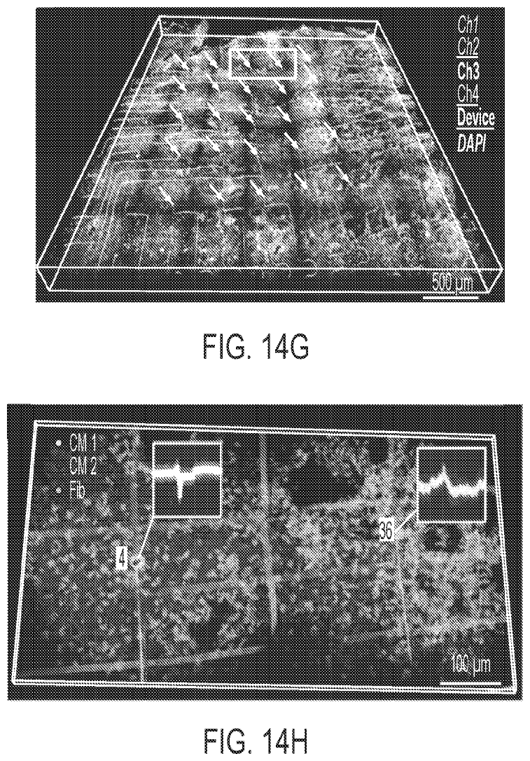

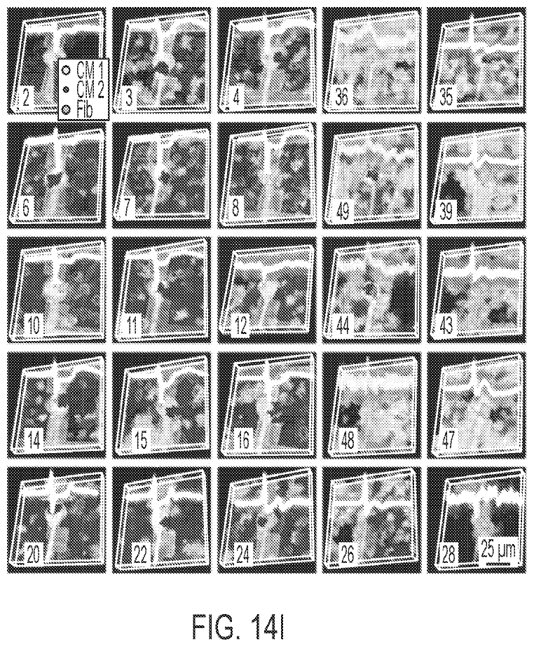

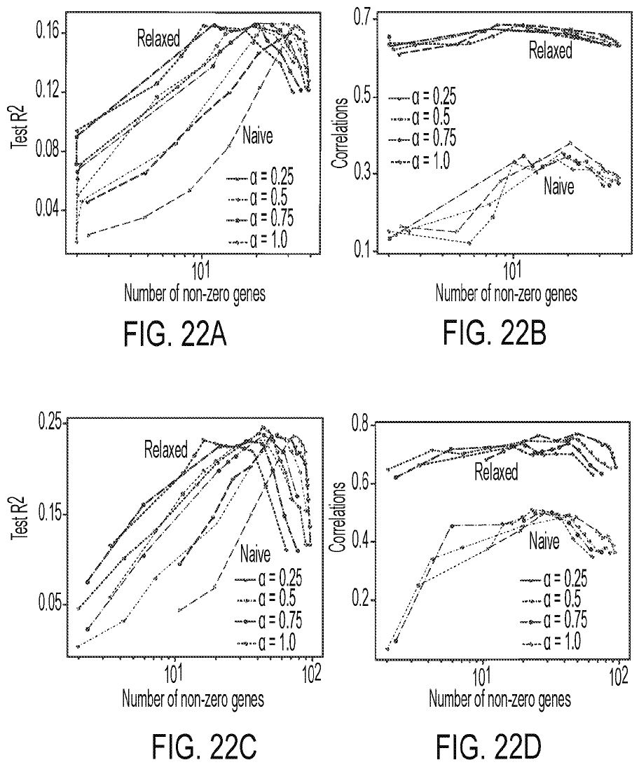

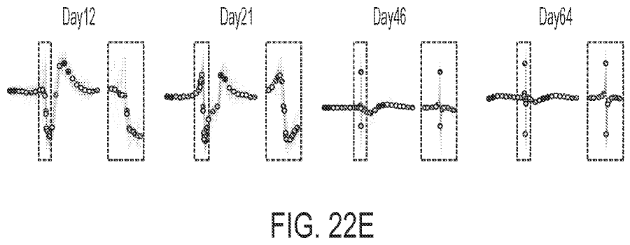

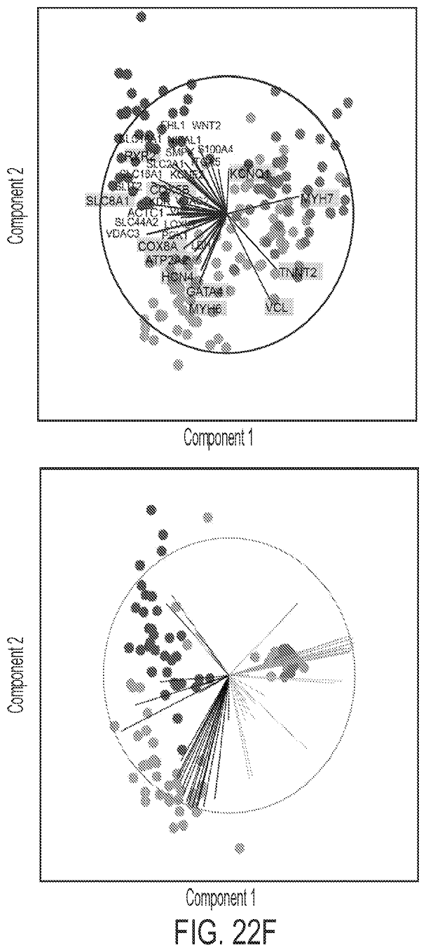

A unique capability of in situ electro-seq is to use continuous single-cell electrical measurement to predict and infer a continuous mapping of gene expression from the same tissue during development and function. To examine to what extent the t-state can be predicted by e-state during cardiac maturation, sparse reduced-rank regression (RRR)⁴² was applied to visualize the gene-to-electrophysiology relationship. 62 electrophysiological features from each cell were used (FIG. 14A and FIG. 22E). Cross-validation was performed by tuning the regularization strength (FIGS. 22A-22D). The selected model chose 32 genes with a 5-dimensional latent space and achieved a cross-validated R² of 0.2 for CM-correlated genes. Then, to visualize the structure of the latent space, gene expression and electrophysiological features were projected onto the latent dimensions (FIG. 22F). The cross-validated correlation between the first two pairs of projections was 0.72 and 0.49respectively. These first two components separate CM groups by their days of differentiation. These different groups show distinct correlated genes with electrophysiology features. The model detected cardiac structural related genes such as myosin heavy chain (MYH6, MYH7), troponin complex (TNNT2), and Z-disc (VCL, VIM) as well as calcium signaling gene (RYR2) and mitochondrial genes (COX5B, COX8A) (FIG. 14C and FIG. 22F). In addition, an RRR model restricted to using only ion channel genes also achieved R²=0.15, and correlations of 0.68 and 0.37 in the first two pairs of projection (FIG. 14B). Genes detected by the ion-channel-only model are endoplasmic reticulum calcium transporting gene (ATP2A2), sodium-calcium exchanger (SLC8A1), potassium channel (KCNK6, KCNQ1, KCND3), Cyclic nucleotide-gated ion channel (HCN4) (FIGS. 14B-14C), whose molecular functions match with the previous knowledge of action potential waveform⁴³⁻⁴⁴.

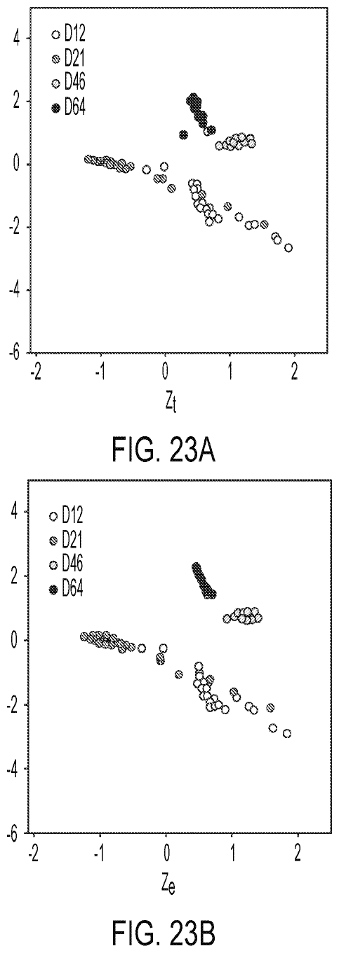

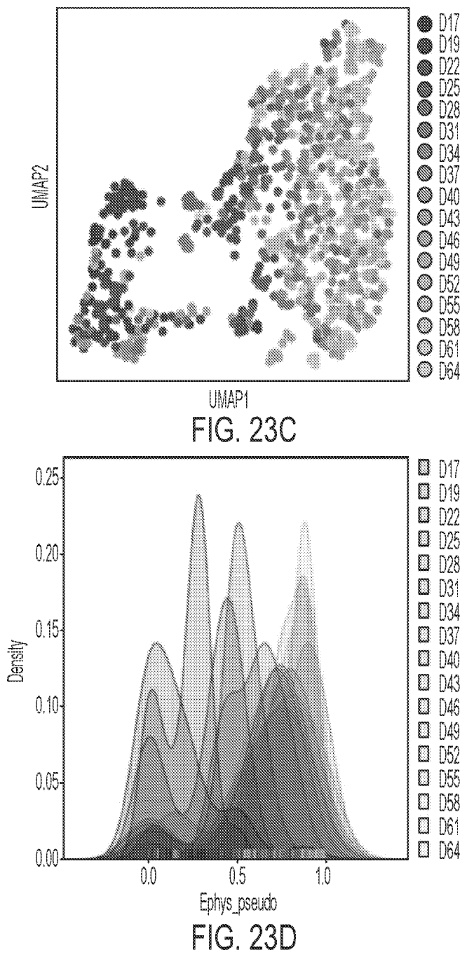

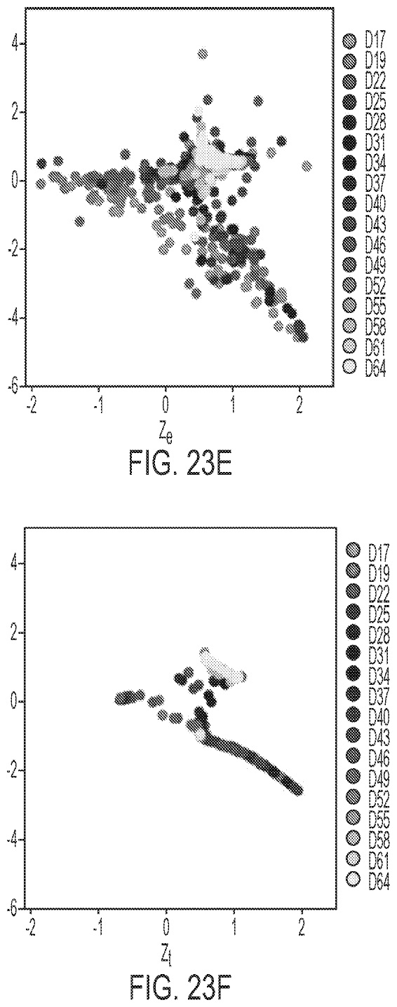

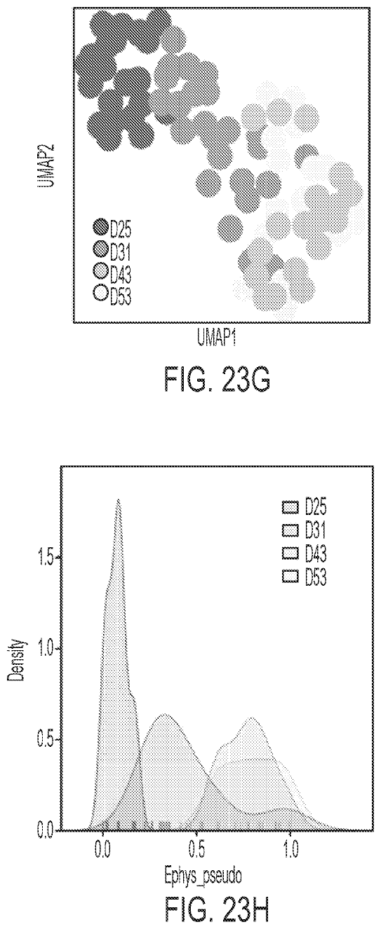

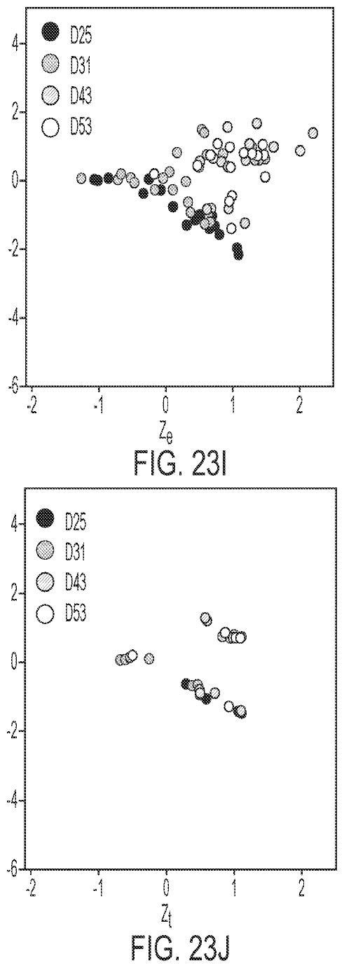

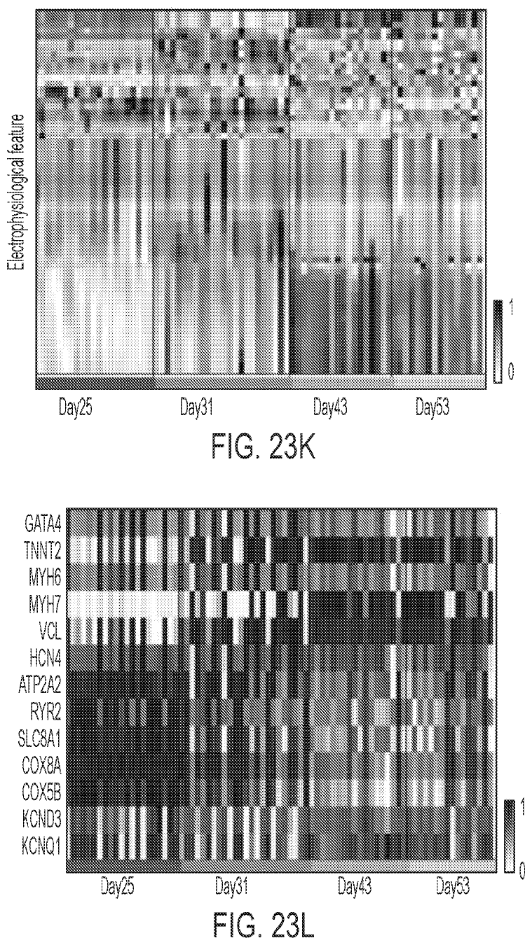

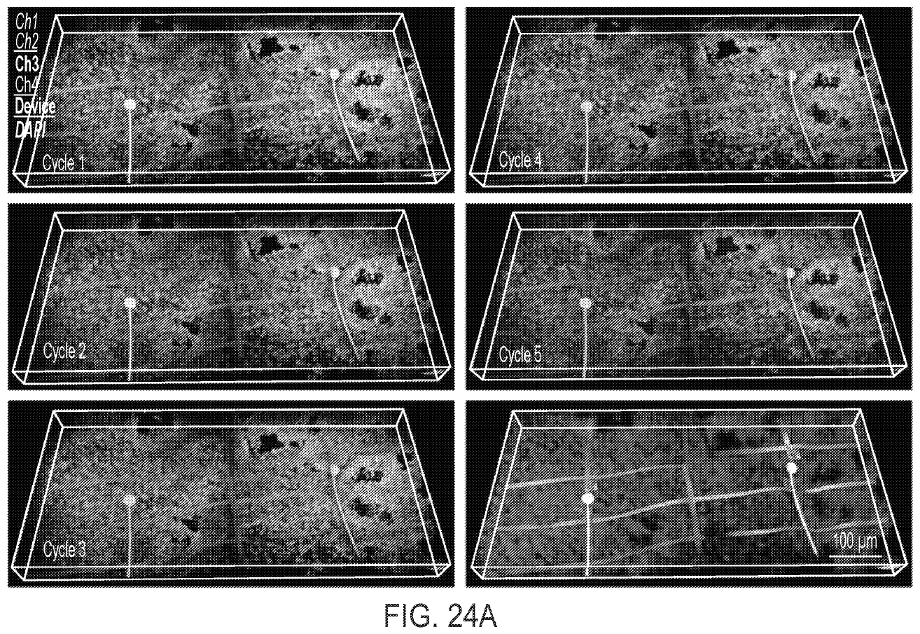

To enable electrophysiology-to-gene prediction, a coupled autoencoder⁴⁵⁻⁴⁶ was used to learn coordinated representations of integrative electrophysiology and gene expression data from Day 12, Day 21, Day 46, and Day 64 of differentiation generated by in situ electro-seq (FIG. 14D). Aligned 2D representations Zt and Ze for the high-dimensional gene expression and electrophysiology data Xt and Xe encoded from the encoder ε (FIGS. 23A-23B) showed well separated and aligned gene expression and electrophysiology distributions. This result suggests a common latent representation exists across gene expression and electrophysiological data so that in situ electro-seq-generated electrophysiology data can be used to predict gene expression. Then, the coupled autoencoder was applied to the electrical measurement recorded every three days of a hiPSC-CM patch over the time course of maturation from Day 17 to Day 64 of differentiation (FIG. 14E and FIGS. 23C-23D). The predicted single-cell gene expression profile shows the evolution of electrophysiology-related genes identified by RRR models (FIG. 14F, FIGS. 23E-23F). The coupled autoencoder was further applied to the electrical measurement of a millimeter-scale hiPSC-CM organoid with mesh electronics fully embedded across the 3D volume (FIGS. 23G-23J). The predicted single-cell gene expression profile shows the evolution of electrophysiology-related genes with much higher variation at the single-cell level (FIGS. 23K-23L), suggesting the intrinsic heterogeneity in hiPSC-CM maturation in 3D organoids⁴⁴.

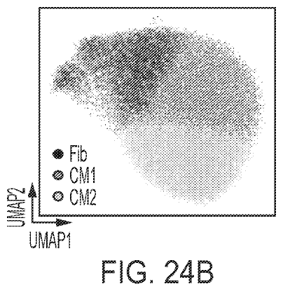

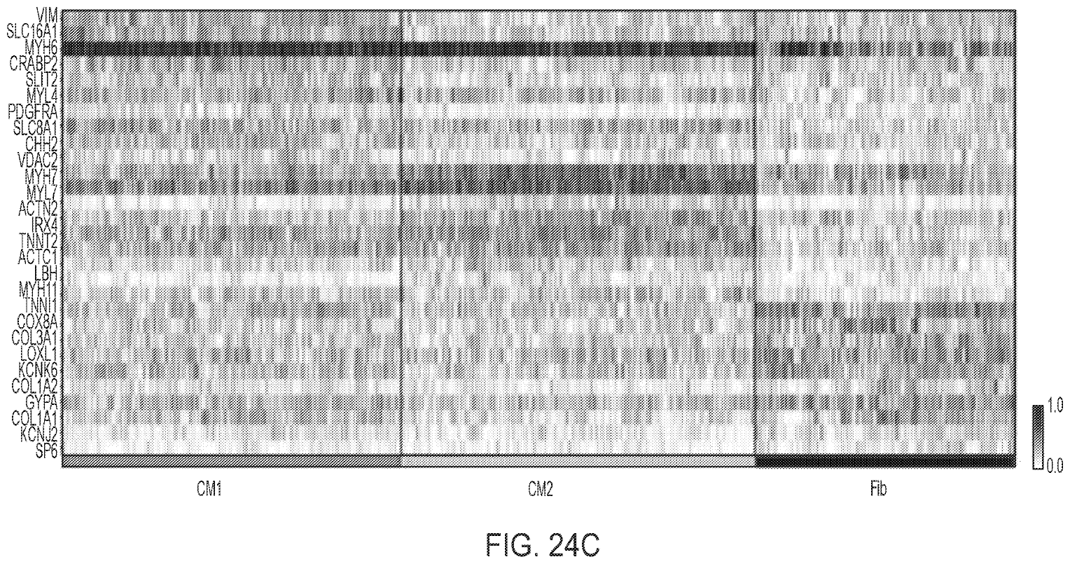

In situ electro-seq was applied to the heterogeneous cardiac patch formed by hiPSC-CMs at different maturation stages (FIG. 14G and FIGS. 24A-24C). The results showed accurate mapping of gene expression and electrophysiology heterogeneity at single-cell resolution from the sample (FIGS. 14H-14I), suggesting its applicability to tissues with spatially heterogeneous cell types and states.

Overall, this example demonstrates that in situ electro-seq is capable of integrating electrophysiology and gene expression at the single-cell level, providing (i) multimodal joint cell clustering in spatially heterogenous tissue at different stages of hiPSC-CM maturation, which is challenging to directly trace by previous approaches; (ii) cross-modal correlations and predictions that use continuous electrical measurement to predict and infer single-cell gene expression evolution from tissues; and (iii) identification of gene programs directly relevant to electrophysiology maturation. In situ electro-seq may be applied to in vivo tissues from animals to map single-cell gene expression and functions from cardiac and neural systems as well as their pathophysiological states in which tissue-wide electrophysiological dysfunctions are related to cell-level gene expression variations, such as neuropsychiatric diseases⁴⁷⁻⁴⁸ (e.g., autism spectrum disorder, bipolar disorders, etc.) and cardiac arrhythmia⁴⁹ (e.g., atrial fibrillation, ventricular tachycardia, etc.).

### Methods

### 1. Stretchable Mesh Nanoelectronics

Fabrication of stretchable mesh electrode array. Fabrication of the ultra-flexible, stretchable mesh nanoelectronics was based on methods described previously^{3, 29-30}. Key steps are described as follows: 4-inch glass wafers (Soda lime glass) were used as a transparent and insulating substrate for fabrication and cell culture. The glass wafers were cleaned by piranha solution (3:1 mixture of sulfuric acid and 30% hydrogen peroxide), followed by rinsing with deionized (DI) water and drying with N₂. Hexamethyldisilazane (HMDS, MicroChem) was spin-coated at 4000 rpm to increase adhesion of photoresists with the substrate. LOR 3A (300 nm, MicroChem)/S1805 (500 nm, MicroChem) were spin-coated at 4000 rpm/4000 rpm, followed by baking at 180° C. for 5 mins and at 115° C. for 1 min, respectively. Ni sacrificial layer was exposed by using a Karl Suss MA6 mask aligner with 365 nm ultraviolet (UV) light at 40 mJ/cm² and developed by CD-26 developer (MICROPOSIT) for 70 s. O₂ plasma (Anatech Barrel Plasma System) was used for the removal of photoresist residues at 50 W for 30 s. Sharon Thermal Evaporator was used for the deposition of 100-nm-thick Ni followed by a standard lift-off procedure in remover PG (MicroChem) for 2 hours. After patterning the Ni layer, SU-8 precursor (SU-8 2000.5, MicroChem) was spin-coated at 4000 rpm, pre-baked at 65° C./95° C. for 2 mins each, exposed to 365 nm UV at 200 mJ/cm², post-baked at 65° C./95° C. for 2 mins each, developed using SU-8 developer (MicroChem) for 60 s, rinsed by isopropyl alcohol (IPA) for 30 s, dried by N₂ gun, and hard-baked at 180° C. for 40 mins to define mesh-like SU-8 400-nm-thick patterns as the bottom encapsulation layer. After patterning the SU-8 bottom layer, HMDS/LOR3A/S1805 photoresist layers were spincoated as described above, followed by depositing 5/40/5-nm-thick chromium/gold/chromium (Cr/Au/Cr) by the electron-beam evaporator (Denton), and the standard lift-off procedure in the remover PG (MicroChem) overnight to define the Cr/Au interconnects. Then, the same photolithography process was used to define 5/50-nm-thick chromium/platinum (Cr/Pt) as electrodes. After patterning electrodes, the top SU-8 encapsulating layer was patterned using the same method described for patterning the bottom SU-8 layer. Finally, fluorescent E-barcodes were defined by patterning the SU-8 structure doped by adding 0.004 wt % of Rhodamine 6G powder (Sigma-Aldrich) into SU-8 precursor.

Connection of stretchable mesh electrode array with flexible cable for electrical recording. Next, the flexible flat cable (FFC, Molex) was soldered onto the input/output pads using a flip-chip bonder (Finetech Fineplacer), followed by glueing a culture chamber onto the substrate wafer to completely enclose the mesh part of the device using a biocompatible adhesive (Kwik-Sil, WPI). Then, Pt black (PtB) was electroplated on the Pt electrode array using a precursor of 0.08 wt % chloroplatinic acid (H₂PtCl₆) solution (Sigma-Aldrich) in H₂O. The precursor was drop-casted onto the device, followed by passage of a 1 mA/cm² DC electric current density for 3 mins using mesh electrodes as anodes and an external Pt wire as the cathode¹⁸. The device was then rinsed with DI water for 30 s and dried by N₂. Finally, the surface of the device was treated with oxygen plasma (Anatech 106 oxygen plasma barrel asher), followed by adding 1 mL of Ni etchant (type TFG, Transene) into the chamber for 2 to 4 hours to completely release the mesh electronics from the glass substrate. The device was then ready for subsequent sterilization steps before cell culture.

Electrochemical measurements. The electrochemical impedance spectra (EIS) of the electrodes were measured based on methods described previously⁴⁹. The three-electrode setup was used to measure the EIS of each electrode. A standard silver/silver chloride (Ag/AgCl) electrode and platinum wire (300 μm in diameter, 1.5 cm in length immersed) were used as reference electrode and counter electrode, respectively. The device was immersed in 1×PBS solution (Thermofisher) during measurement. The SP-150 potentiostat (Bio-logic) along with its commercial software EC-lab was used to perform the measurements. For each measurement, at least three frequency sweeps were measured from 1 MHz down to 1 Hz to obtain statistical results. A sinusoidal voltage of 100 mV peak-to-peak was applied. For each data point, the response to 10 consecutive sinusoids (spaced out by 10% of the period duration) was accumulated and averaged.

### 2. Cell Culture, Tissue Integration, and Electrical Recording

Cell culture and cardiomyocytes (CMs) differentiation. Human induced pluripotent stem cells (hiPSC, hiPSC-IMR90-1) were obtained from WiCell Research Institute (Madison, WI, USA). Authentication and testing for mycoplasma were performed by WiCell Research Institute. IMR90-1 cells were cultured on a Matrigel-coated 6-well plate with Essential 8 medium (Gibco). The medium was changed daily. The cells were passaged every 3-4 days. hiPSC-derived cardiomyocytes were generated according to the methods described previously^{37,51}. The IMR90 cells were cultured on a Matrigel-coated 6-well plate with Essential 8 medium to 70-80% confluency before initiating cardiac differentiation. The first day was defined as day 0. For cardiac differentiation, the cells were maintained in RPMI 1640 medium (Gibco) plus 1% B27-insulin (Gibco). CHIR99021 (12 mM; BioVision) was applied on day 0; IWR1 (5 mM; Cayman) was applied from day 3 to day 4. The cardiac cells were maintained in RPMI 1640 medium plus 1% B27 (Gibco) from day 7 and the medium was changed every other day accordingly.

Integration of mesh electronics with hiPSC-CM patch. First, the released stretchable mesh electronics in the culture chamber was rinsed with DI water, decontaminated by 70% ethanol, and incubated with Poly-D-lysine hydrobromide (0.01% w/v) overnight followed by coating with Matrigel solution (100 μg/mL) for about 1 hour at 37° C. Then, the device was pre-chilled on an ice bag and 70 μL Matrigel solution (10 mg/mL) was added from the edge of the chamber to the cell culture medium, ensuring that the Matrigel covered the entire bottom substrate of the cell culture chamber underneath the stretchable mesh electronics. Next, the device was incubated for at least 30 mins at 37° C. to cure the Matrigel solution into a Matrigel hydrogel layer. Finally, hiPSC-CMs were incubated with 0.05% Trypsin-EDTA solution (Biosciences) for 5 mins and then dissociated into single cells. About 3-4 million cells were suspended in 1 mL RPMI 1640 medium plus 1% B27 and then transferred onto the cured electronics/Matrigel hybrids in the cell culture chamber and maintained at 37° C., 5% CO². 5μM rock inhibitor (Y27632) was added to the medium on the first day to improve cell viability. The CMs formed a continuous cell patch with the stretchable mesh electronics embedded within 24-48 hours. Notably, to include cells with different differentiation stages, ca. 2 million Day 18 of differentiation CMs were seeded from one side of the culture chamber and cultured for 5 days; then, another ca. 2 million Day 7 of differentiation CMs were seeded from the opposite side of the cell culture chamber.

Electrophysiological measurement. The Blackrock CerePlex Direct voltage amplifier along with a 32 channel Blackrock μ digital headstage connected to the device were used to record electrical activity from the cardiac patch. The culture medium was grounded by a Pt electrode. A second Pt electrode was used as a reference electrode. During electrical measurement, samples were placed on a battery powered warming plate that maintained thermostatic 37° C. The measurement setup was placed into a Faraday cage. A sampling rate of 30,000 Hz was used for the electrical recording. The cell electrical activities were recorded every 3 days. MATLAB and Python codes provided by Blackrock were used to load, view, and convert raw data files into an accessible format for data analysis.

### 3. In Situ Sequencing

In situ sequencing experiments were performed based on methods described previously with some modifications. Glass-bottom 12-well plates (Mattek, P12G-1.5-14-F) were first treated with oxygen plasma for 5 mins (Anatech Barrel Plasma System, 100 W, 40% O₂) followed by methacryloxypropyltrimethoxysilane (Bind-Silane) solution (88% ethanol, 10% acetic acid, 1% Bind-Silane, 1% H₂O) treatment for 1 hour. The 12-well plates were then rinsed with ethanol for 3 times and were left to dry at room temperature (R.T.) for 3 hours. The 12-well plates were further treated with 0.1 mg/mL of Poly-D-lysine solution for 1 hour at R.T. and rinsed 3 times with H2O. The plates were air-dried for 1 hour at R.T. Micro cover glasses (12 mm) were pretreated with Gel Slick at R.T. for 10 mins and were then air-dried before using.

The cardiac tissue was fixed with 1 mL 1.6% PFA for 30 mins at R.T. and then washed with PBS 3 times for 10 mins each time. The sample was then transferred from the chamber to the 12-well plates and permeabilized with 1 mL (0.1 M glycine, 0.1 U/μL SUPERase·In, 0.5% Triton-X 100 in PBS) for 30 mins. The sample was washed with 1 mL PBST (0.1% Triton-X 100 in PBS) 3 times for 10 mins each. The sample was then incubated in 1× hybridization buffer (2× SSC, 10% formamide, 1% Triton-X 100, 20 mM RVC, 0.1 mg/mL yeast tRNA and pooled SNAIL probes at 20 nM per oligo) in a 40° C. humidified oven with gentle shaking for 48 hours. The sample was washed with 1 ml PBSTV (1% RVC in PBST) at 37° C. 3 times for 20 mins each and washed with high salt buffer (4× SSC in PBST) for another 20 mins at 37° C., and then washed with PBST three times for 10 mins each at R.T. The sample was then incubated in 1 mL ligation mixture (1:50 T4 DNA ligase, 1:100 BSA, 0.2 U/μL SUPERase-In) at R.T. overnight and then washed with 1 mL PBST three times for 10 mins each. The sample was incubated in 1 mL RCA mixture ((1:50 Phi29 DNA polymerase, 250 μM dNTP, 1:100 BSA, 0.2 U/μL SUPERase-In and 20 μM 5-(3-aminoallyl)-dUTP) at 4° C. for 1 hour before incubating at 30° C. for 6 hours and then washed with 1 mL PBST 3 times for 10 mins each. The sample was incubated with 20 mM acrylic acid NHS ester in PBST for 3 hours at R.T. and washed with PBST 3 times for 10 mins each. The sample was then incubated with monomer buffer (4% acrylamide, 0.2% bis-acrylamide, 2× SSC) overnight at R.T. The buffer was then aspirated and 55 μL of polymerization mixture (0.2% ammonium persulfate, 0.2% tetramethylethylenediamine dissolved in monomer buffer) was added to the sample. The Gel Slick coated coverslip was immediately put on the sample and polymerization was conducted in an N2 container for 90 mins. The sample was then washed with PBST 3 times for 10 mins each.

Five cycles of sequencing experiments were performed to decode gene identity. Within each cycle, the sample was first treated with a stripping buffer (60% formamide, 0.1% Triton-X-100) at R.T. 6 times, 15 mins each followed by PBST wash for 6 times, 10 mins each. Then the sample was incubated with the sequencing mixture (1:25 T4 DNA ligase, 1:100 BSA, 10 μM reading probe and 5 μM fluorescent oligos) at R.T. for 12 hours. Then the sample was washed with the washing and imaging buffer (2XSSC, 10% formamide and 0.1% Triton-X-100) 5 times, 10 mins each. DAPI was dissolved in PBST and used for nuclei staining for 20 mins. Finally, the sample was immersed in the washing and imaging buffer for imaging. Image acquisition was performed with Leica TCS SP8 confocal microscopy with 25× water-immersion objective (NA 0.95), with voxel size of 230 nm×230 nm×570 nm.

### 4. Data Analysis

In situ sequencing analysis. A customized computational pipeline was built with MATLAB (2019b) to decode gene identity and quantify the gene expression level of each cell from the in situ sequencing images. First, sequencing fluorescence images were preprocessed with top-hat filtering by a disk structuring element (radius=3) to remove the background noise. Second, the contrast of the image for each channel from the second to fifth sequencing cycle was adjusted to match the image from the first cycle with the histogram matching function “imhistmatchn”. Third, the composite fluorescence images for the second to fifth cycle were registered with the composite fluorescence image from the first cycle using a phase correlation algorithm followed by local distortion registration with function “imregdemons”. Fourth, the dots of amplicon locations were identified from images in the first cycle by a 3D regional maximum detection algorithm implemented in function “imregionalmax”. Then the dominant color of every identified dot in each cycle was determined by a 3×3×3 voxel volume surrounding its centroid location. The color sequence for each dot was decoded as a gene barcode and compared with the code-book. Fifth, cell segmentation was performed with ClusterMap³⁴ or Stardist³⁶ with custom cell mask dilation method, then RNA reads were assigned to the segmented cells accordingly.

Python package Scanpy v1.6.0⁵² was used for single-cell gene expression analysis. Cells expressing less than 40 gene counts or only expressing three kinds of gene were filtered out. Gene counts of each cell were normalized so that the total count of all genes in each cell equals the median number of total counts across all cells. The normalized count value is then log-transformed with log2(x+1). Combat⁵³ was used to remove the potential batch effect among different imaging positions. Each gene in the cell-by-gene matrix was scaled to unit variance and zero mean followed by dimensionality reduction with principal components analysis (PCA). Based on the explained variance ratio, the top principal-components were used to construct the k nearest neighbor (kNN) graph for Leiden clustering³⁴. Uniform Manifold Approximation and Projection (UMAP)²⁷ was used to visualize the 2D representation of each cell. Monocle 3⁴⁰ is used to compute pseudotime along the cell trajectory.

Electrically recorded cell identification. Electrode position was located using the 3D electrode image collected by reflection-mode imaging and identified by the E-barcode positions. The electrode position in x and y coordinate was determined by the following steps: the electrode image was first projected to the x-y plane by maximum intensity projection (MIP) and transferred to gray-scale (pixel value ranging from 0-255). Then the MIP image was filtered with a global threshold of 50 to remove the non-electrode background. A 201-by-201 pixel size gaussian filter was applied to adaptively filter out the non-circular area, which is the I/O connect of the electrode. After locating the electrode in the x-y plane, the z coordinate of the electrode position was determined by fitting a 2D linear plane surface. The electrode recorded cell was further determined by calculating the area of intersection between each neighborhood CM cell and the electrode. The cell with the largest intersection area was identified as the electrically recorded cell.

Electrophysiology data processing. The procedure of SpikeInterface⁵⁴ was followed to detect the spikes which passed the threshold in one channel. Each spike has a fixed length of 1.6 second with the sampling rate of 10 kHz. After spike detection, the spikes were aligned at the minimum of the corresponding spike dv/dt, and then averaged to get a spike representation of that channel.

For each spike representation, the spike was denoised with wavelet denoising using PyWavelets⁵⁵. Then, the spike features were extracted through two cycles of down sampling operations. The whole 1.6 second length spike was down sampled to 20 points (zoomed out binning) and then down sampled the 0.15 second length spike near the minimum of the differentiated spike to 42 points (zoomed in binning). In total, 62 feature points were generated for each spike representation of each channel.

Weighted Nearest Neighbor (WNN). The weighted nearest neighbor (WNN)³⁹, i.e. FindMultiModalNeighbors function from Seurat v4⁴⁰, was used to integrate the gene expression and electrophysiology data collected by in situ electro-seq. The principle component dimension for gene expression and electrophysiology was set as 7 and 6 (the elbow point in PCA variance), respectively. k=20 was used to find the k nearest neighbor and calculate the modality-specific weights. A WNN graph was then built for downstream analysis such as joint clustering and pseudotime analysis

Gene expression, electrophysiology and WNN joint pseudotime analysis. An R package Monocle3 was used for the pseudotime calculation of gene expression, electrophysiology features, and the WNN joint representation described above. A set of hyperparameters (Euclidean distance ratio=1, geodesic distance ratio=⅔, minimal branch length =5) in function learn-graph was used to first learn a principle graph of maturation. The node at the position of earliest stage was manually chosen as the root of the principle graph to finalize the trajectory. Then the function order_cells was used to calculate the pseudotime.

Sparse reduced-rank regression (RRR) model and bibiplot. For the RRR analysis⁴¹, 62 electrophysiological features were used across all 162 electrically recorded CMs. Both electrophysiological features and gene expression were normalized and z-scored as described above. Ninety-seven CM-correlated genes and ion channel genes were selected for the RRR analysis.

In brief, RRR finds a linear mapping of gene expression levels to a low-dimensional latent representation, from which the electrophysiological features are then predicted with another linear transformation. In FIGS. 14A-14B, a model with rank r=3, ridge penalty (α=0.5) and lasso penalty (λ=1.5) was used to yield a selection of 12 genes. In FIGS. 22E-22F, a model with rank r=5, ridge penalty (α=0.5), and lasso penalty (λ=1.5) was used to yield a selection of 32 genes. In FIGS. 22A-22D, cross-validation was done by using 10 folds, elastic net α-values 0.5, 0.75, and 1.0, and k-values from 0.2 to 6.0.

Coupled autoencoder prediction model. Coupled autoencoder⁴⁴⁻⁴⁵ was used for cross-modal prediction. Specifically, hyperparameter (latent loss weight=1, Adam optimizer with learning rate=0.0001, batch size=10, training epoch=100, epoch step=1000) and latent dimensionality (d=2) were used to capture the variability in the dataset. After training the coupled autoencoder network with the data collected in Day 12, Day 21, Day 46, and Day 64, continuously recorded electrophysiology data z-scored by the method mentioned above was used to infer continuous gene expression profiles for all the electrically recorded cells.

### Example 3

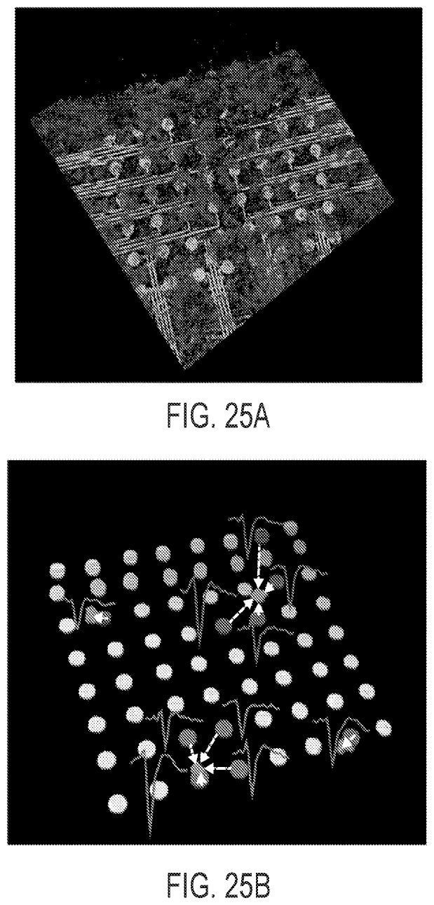

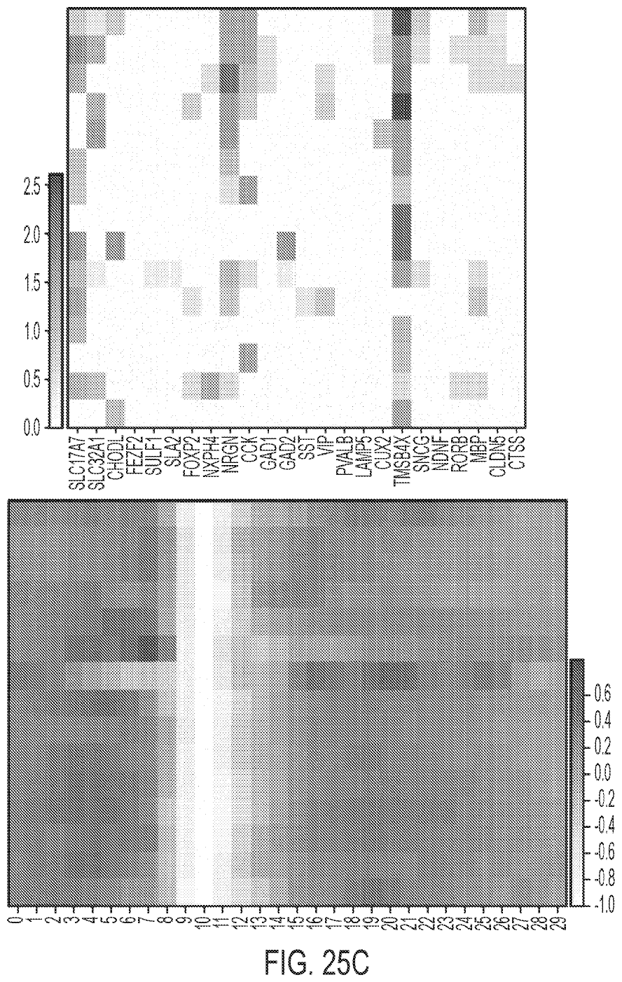

The in situ electro-seq methods described herein were also applied to the neural system. In situ electro-seq was performed to analyze expression of >1000 genes with a full view of the entire neural tissue-electronics hybrid (FIG. 25A). 3D neuron identification was performed by spike detection from a multiple electrode array (FIG. 25B). The identified neurons and corresponding electrodes are shown with recorded single-unit action potential overlapped. Features were extracted from the waveform of averaged spikes, and the corresponding highest differentially expressed genes expressed in the electrically recorded neurons were identified (FIG. 25C).

### REFERENCES

All of the following references are each incorporated herein by reference in their entireties.
1. Lian, X. et al. Robust cardiomyocyte differentiation from human pluripotent stem cells via temporal modulation of canonical Wnt signaling. Proc. Natl. Acad. Sci. USA 109, E1848—E1857 (2012).
2. Lee, J. et al. Activation of PDGF pathway links LMNA mutation to dilated cardiomyopathy. Nature 572, 335-340 (2019).
3. Li, Q. et al. Cyborg organoids: implantation of nanoelectronics via organogenesis for tissue-wide electrophysiology. Nano Lett. 19, 5781-5789 (2019).
4. Ma, Z. et al. Self-organizing human cardiac microchambers mediated by geometric confinement. Nat. Commun. 6, 7413 (2015).
5. Takebe, T. et al. Vascularized and complex organ buds from diverse tissues via mesenchymal cell-driven condensation. Cell Stem Cell 16, 556-565 (2015).
6. Asakura, K. et al. Improvement of acquisition and analysis methods in multi-electrode array experiments with iPS cell-derived cardiomyocytes. J. Pharmacol. Toxicol. Methods 17-26 (2015).
7. Lein, E., Borm, L. E. & Linnarsson, S. The promise of spatial transcriptomics for neuroscience in the era of molecular cell typing. Science 358, 64-69 (2017)
8. Wang, X. et al. Three-dimensional intact-tissue sequencing of single-cell transcriptional states. Science 361, eaat5691 (2018).
9. Becht, E. et al. Dimensionality reduction for visualizing single-cell data using UMAP. Nat. Biotechnol. 37, 38-44 (2018).
10. Friedman, C. E. et al. Single-cell transcriptomic analysis of cardiac differentiation from human PSCs reveals HOPX-dependent cardiomyocyte maturation. Cell Stem Cell 23, 586-598.e8 (2018).
11. Vercauteren, T., Pennec, X., Perchant, A. & Ayache, N. Diffeomorphic demons: efficient non-parametric image registration. NeuroImage 45, S61-S72 (2009).
12. Weigert, M., Schmidt, U., Haase, R., Sugawara, K. & Myers, G. Star-convex polyhedra for 3D object detection and segmentation in microscopy. in 2020 IEEE Winter Conference on Applications of Computer Vision (WACV) (IEEE, 2020).
13. Zhang, J. et al. Functional cardiac fibroblasts derived from human pluripotent stem cells via second heart field progenitors. Nat. Commun. 10, 2238 (2019).
14. Alivisatos, A. P. et al. The brain activity map. Science 339, 1284-1285 (2013).
15. Fuzik, J. et al. Integration of electrophysiological recordings with single-cell RNA-seq data identifies neuronal subtypes. Nat Biotechnol 34, 175-183 (2016).
16. Cadwell, C. R. et al. Electrophysiological, transcriptomic and morphologic profiling of single neurons using Patch-seq. Nat Biotechnol 34, 199-203 (2016).
17. Christoph, J. et al. Electromechanical vortex filaments during cardiac fibrillation. Nature 555, 667-672 (2018).
18. Weber, M. et al. Cell-accurate optical mapping across the entire developing heart. Elife 6, e28307 (2017).
19. Huang, J., Sheng, J. & Wang, D. Manifold learning analysis suggests novel strategies for aligning single-cell multi-modalities and revealing functional genomics for neuronal electrophysiology. bioRxiv, 2020.2012.2003.410555 (2021).
20. Jun, J. J. et al. Fully integrated silicon probes for high-density recording of neural activity. Nature 551, 232-236 (2017).
21. Abbott, J. et al. A nanoelectrode array for obtaining intracellular recordings from thousands of connected neurons. Nat Biomed Eng 4, 232-241 (2020).
22. Viventi, J. et al. Flexible, foldable, actively multiplexed, high-density electrode array for mapping brain activity in vivo. Nat Neurosci 14, 1599-1605 (2011).
23. Tang, F. et al. mRNA-Seq whole-transcriptome analysis of a single cell. Nat Methods 6, 377-382 (2009).
24. Stuart, T. & Satija, R. Integrative single-cell analysis. Nat Rev Genet 20, 257-272 (2019). Regev, A. et al. The Human Cell Atlas. Elife 6 (2017).
26. Xu, S. et al. Behavioral state coding by molecularly defined paraventricular hypothalamic cell type ensembles. Science 370 (2020).
27. Kodandaramaiah, S. B., Franzesi, G. T., Chow, B. Y., Boyden, E. S. & Forest, C. R. Automated whole-cell patch-clamp electrophysiology of neurons in vivo. Nat Methods 9, 585-587 (2012).
28. Liu, J. et al. Multifunctional three-dimensional macroporous nanoelectronic networks for smart materials. Proc Natl Acad Sci U S A 110, 6694-6699 (2013).
29. Liu, J. et al. Syringe-injectable electronics. Nat Nanotechnol 10, 629-636 (2015).
30. Li, Q. et al. Cyborg organoids: implantation of nanoelectronics via organogenesis for tissue-wide electrophysiology. Nano Lett 19, 5781-5789 (2019).
31. Tian, B. et al. Macroporous nanowire nanoelectronic scaffolds for synthetic tissues. Nat Mater 11, 986-994 (2012).
32. Feiner, R. et al. Engineered hybrid cardiac patches with multifunctional electronics for online monitoring and regulation of tissue function. Nature Materials 15, 679-685 (2016).
33. Cui, Y. et al. Single-cell transcriptome analysis maps the developmental track of the human heart. Cell Rep 26, 1934-1950 e1935 (2019).
34. He, Y. et al. ClusterMap: multi-scale clustering analysis of spatial gene expression. BioRxiv 2021, doi:10.1101/2021.02.18.431337 (2021).
35. Traag, V. A., Waltman, L. & van Eck, N. J. From Louvain to Leiden: guaranteeing well-connected communities. Sci Rep 9, 5233 (2019).
36. Weigert, M., Schmidt, U., Haase, R., Sugawara, K. & Myers, G. in 2020 IEEE Winter Conference on Applications of Computer Vision (WACV) 3655-3662 (2020).
37. Lian, X. et al. Robust cardiomyocyte differentiation from human pluripotent stem cells via temporal modulation of canonical Wnt signaling. Proc Natl Acad Sci U S A 109, E1848-1857 (2012).
38. Churko, J. M. et al. Defining human cardiac transcription factor hierarchies using integrated single-cell heterogeneity analysis. Nature Communications 9 (2018).
39. Scala, F. et al. Phenotypic variation of transcriptomic cell types in mouse motor cortex. Nature (2020).
Hao, Y. et al. Integrated analysis of multimodal single-cell data. BioRxiv, doi:10.1101/2020.10.12.335331 (2020).
41. Cao, J. et al. The single-cell transcriptional landscape of mammalian organogenesis. Nature 566, 496-502 (2019).
42. Kobak, D. et al. Sparse reduced-rank regression for exploratory visualization of multimodal data sets. BioRxiv 2020, 10.1101/302208 (2020).
43. Garg, P. et al. Human induced pluripotent stem cell-derived cardiomyocytes as models for cardiac channelopathies. Circulation Research 123, 224-243 (2018).
44. Karbassi, E. et al. Cardiomyocyte maturation: advances in knowledge and implications for regenerative medicine. Nature Reviews Cardiology 17, 341-359 (2020).
45. Wang, W., Arora, R., Livescu, K. & Bilmes, J. in Proceedings of the 32nd International Conference on Machine Learning Vol. 37 (eds Bach Francis & Blei David) 1083-1092 (PMLR, Proceedings of Machine Learning Research, 2015).
46. Gala, R. et al. Consistent cross-modal identification of cortical neurons with coupled autoencoders. Nature Computational Science 1, 120-127 (2021).
47. Quadrato, G., Brown, J. & Arlotta, P. The promises and challenges of human brain organoids as models of neuropsychiatric disease. Nat Med 22, 1220-1228 (2016).
48. Sanders, S. J. et al. A framework for the investigation of rare genetic disorders in neuropsychiatry. Nat Med 25, 1477-1487 (2019).
49. Liu, J. et al. Intrinsically stretchable electrode array enabled in vivo electrophysiological mapping of atrial fibrillation at cellular resolution. Proc Natl Acad Sci U S A 117, 14769-14778 (2020).
50. Feltham, A. & Spiro, M. Platinized platinum electrodes. Chem. Rev. 71, 177-193 (1971).
51. Lian, X. et al. Directed cardiomyocyte differentiation from human pluripotent stem cells by modulating Wnt/f3-catenin signaling under fully defined conditions. Nat. Protoc. 8, 162-175 (2013).
52. Wolf, F. A., Angerer, P. & Theis, F. J. SCANPY: large-scale single-cell gene expression data analysis. Genome Biol. 19, 1-5 (2018).
53. Johnson, W. E., Li, C. & Rabinovic, A. Adjusting batch effects in microarray expression data using empirical Bayes methods. Biostatistics 8, 118-127 (2007).
54. Buccino, Alessio P., et al. SpikeInterface, a unified framework for spike sorting. Elife 9 (2020): e61834.
55. Lee, Gregory, et al. PyWavelets: A Python package for wavelet analysis. Journal of Open Source Software 4.36 (2019): 1237.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub—range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following embodiments.

What is claimed is:

1. A method for correlating a continuous physiological process and a biomolecular process in cells in a tissue, the method comprising steps of:
(a) embedding one or more nanoelectronic devices in the tissue to form a nanoelectronics-tissue hybrid, wherein each nanoelectronic device comprises at least one sensor with a unique electronic barcode;
(b) performing a continuous physiological measurement on the cells;
(c) fixing the nanoelectronics-tissue hybrid;
(d) performing in situ analysis of the biomolecular process on the cells;
(e) performing mapping of the biomolecular process on the cells;
(f) identifying the position of the electronic barcode within the nanoelectronics-tissue hybrid; and
(g) performing cell segmentation to correlate the mapping of the in situ analysis of the biomolecular process with the continuous physiological measurement.

2. The method of claim 1, wherein the continuous physiological process comprises electrophysiological activity.

3. The method of claim 1 or claim 2, wherein the step of performing a continuous physiological measurement comprises performing continuous electrophysiological recording.

4. The method of any one of claims 1-3, wherein the biomolecular process comprises gene expression.

5. The method of any one of claims 1-4, wherein the step of performing in situ analysis of the biomolecular process comprises performing in situ single-cell transcriptome sequencing.

6. The method of any one of claims 1-5, wherein the step of performing mapping of the biomolecular process comprises performing transcriptomic mapping.

7. A method for correlating electrophysiological activity and gene expression in cells in a tissue, the method comprising steps of:
(a) embedding one or more nanoelectronic devices in the tissue to form a nanoelectronics-tissue hybrid, wherein each nanoelectronic device comprises at least one sensor with a unique electronic barcode;
(b) performing continuous electrophysiological recording on the cells;
(c) fixing the nanoelectronics-tissue hybrid;
(d) performing in situ single-cell transcriptome sequencing on the cells;
(e) performing transcriptomic mapping on the cells;
(f) identifying the position of the electronic barcode within the nanoelectronics-tissue hybrid; and
(g) performing cell segmentation to correlate the single-cell transcriptome sequencing data with the electrophysiological recording data.

8. The method of any one of claims 1-7, wherein the electronic barcode is a fluorescence electronic barcode.

9. The method of any one of claims 1-8, wherein the nanoelectronic devices embedded in the tissue comprise over 1000, over 10,000, over 100,000, or over 1,000,000 sensors.

10. The method of any one of claims 1-9, wherein the cells in the tissue comprise over 1,000,000 cells.

11. The method of any one of claims 1-10, wherein the cells in the tissue comprise over 1,000,000,000 cells.

12. The method of any one of claims 1-11, wherein the nanoelectronic devices are tissue-like.

13. The method of any one of claims 1-12, wherein the nanoelectronic devices comprise a polymeric network.

14. The method of any one of claims 1-13, wherein the nanoelectronic devices comprise stretchable mesh.

15. The method of claim 14, wherein the stretchable mesh comprises an overall filling ratio of less than 100%, less than 50%, less than 20%, less than 15%, less than 10%, less than 5%, or less than 1%.

16. The method of claim 14, wherein the stretchable mesh comprises an overall filling ratio of less than 11%.

17. The method of any one of claims 1-16, wherein the nanoelectronic devices are embedded in a serpentine layout, a hexagonal layout, a triangular layout, or a straight layout.

18. The method of any one of claims 1-17, wherein the nanoelectronic devices comprise a mass of less than 50 μg, less than 40 μg, less than 30 μg, less than 20 μg, or less than less than 10 μg.

19. The method of any one of claims 1-18, wherein the nanoelectronic devices comprise a mass of less than 15 μg.

20. The method of any one of claims 1-19, wherein the nanoelectronic devices comprise a top encapsulation layer.

21. The method of claim 20, wherein the top encapsulation layer is an SU-8 encapsulation layer.

22. The method of any one of claims 1-21, wherein the nanoelectronic devices comprise an electrode layer.

23. The method of claim 22, wherein the electrode layer is a platinum electrode layer.

24. The method of claim 22 or 23, wherein the electrode layer comprises a coating.

25. The method of claim 24, wherein the coating is a poly(3,4-ethylenedioxythiophene) coating, a polyaniline coating, or a polypyrrole coating.

26. The method of any one of claims 1-25, wherein the nanoelectronic devices comprise a gold interconnecting layer.

27. The method of any one of claims 1-26, wherein the nanoelectronic devices comprise a bottom encapsulation layer.

28. The method of claim 27, wherein the bottom encapsulation layer is an SU-8 encapsulation layer.

29. The method of any one of claims 1-28, wherein the nanoelectronic devices comprise input and output lines.

30. The method of any one of claims 1-29, wherein the nanoelectronic devices comprise an electrical device, an optical device, a mechanical sensor, a stimulator, or an actuator.

31. The method of any one of claims 1-30, wherein the step of embedding the nanoelectronic devices comprises transferring the nanoelectronic devices onto a two-dimensional sheet of cells and allowing the cells to aggregate, associate, proliferate, and migrate.

32. The method of claim 31, wherein allowing the cells to aggregate, associate, proliferate, and migrate compresses the nanoelectronic devices and embeds them within the cells in the tissue.

33. The method of any one of claims 7-26, wherein the step of performing in situ single cell transcriptome sequencing comprises constructing cDNA amplicons in situ by probe hybridization, enzymatic amplification of the cDNA amplicons, and immobilization of the amplified cDNA in a hydrogel network.

34. The method of claim 33, wherein the cDNA amplicons comprise a gene-specific identifier sequence.

35. The method of claim 34, wherein the gene-specific identifier sequence is read out through fluorescent imaging.

36. The method of any one of claims 7-35, wherein the step of performing in situ single cell transcriptome sequencing comprises performing single cell RNA sequencing.

37. The method of any one of claims 7-36, wherein the step of performing in situ single cell transcriptome sequencing comprises performing spatially-resolved transcript amplicon readout mapping (STARmap).

38. The method of any one of claims 7-37, wherein the step of transcriptomic mapping comprises mapping over 1000 genes simultaneously.

39. The method of any one of claims 7-38, wherein the step of identifying the position of the fluorescence electronic barcode comprises performing confocal microscopy.

40. The method of any one of claims 1-39, wherein the cells are living.

41. The method of any one of claims 1-40, wherein the cells are in vivo.

42. The method of any one of claims 1-41, wherein the tissue is living.

43. The method of any one of claims 1-42, wherein the tissue is in vivo.

44. The method of any one of claims 1-43, wherein the tissue is three-dimensional.

45. The method of any one of claims 1-44, wherein the tissue is a tissue with electrical activity.

46. The method of any one of claims 1-45, wherein the tissue is brain tissue.

47. The method of any one of claims 1-45, wherein the tissue is heart tissue.

48. The method of any one of claims 1-45, wherein the tissue is pancreas tissue.

49. The method of any one of claims 1-45, wherein the tissue is nervous system tissue.

50. The method of any one of claims 1-45, wherein the tissue is muscle tissue.

51. The method of any one of claims 1-45, wherein the tissue is gastrointestinal tract tissue.

52. The method of any one of claims 1-45, wherein the tissue is developing tissue.

53. The method of any one of claims 1-45, wherein the tissue is diseased tissue.

54. The method of any one of claims 1-53, wherein the tissue is an organoid.

55. The method of any one of claims 1-54, wherein the tissue is derived from human induced pluripotent stem cells.

56. The method of any one of claims 1-55, wherein the cells are stem cells.

57. The method of any one of claims 1-56, wherein the cells are progenitor cells.

58. A system comprising one or more nanoelectronic devices within cells in a tissue, wherein each nanoelectronic device comprises at least one sensor with a unique electronic barcode.

59. A system for correlating a continuous physiological process and a biomolecular process in cells in a tissue, wherein the system is prepared by embedding one or more nanoelectronic devices in the tissue to form a nanoelectronics-tissue hybrid, wherein each nanoelectronic device comprises at least one sensor with a unique electronic barcode.

60. A system for correlating electrophysiological activity and gene expression in cells in a tissue, wherein the system is prepared by embedding one or more nanoelectronic devices in the tissue to form a nanoelectronics-tissue hybrid, wherein each nanoelectronic device comprises at least one sensor with a unique electronic barcode.

61. The system of any one of claims 58-60, wherein the electronic barcode is a fluorescence electronic barcode.

62. The system of any one of claims 58-61, wherein over 1000, over 10,000, over 100,000, or over 1,000,000 nanoelectronic devices are embedded within the tissue.

63. The system of any one of claims 58-62, wherein the cells in the tissue comprise over 1,000,000 cells.

64. The system of any one of claims 58-63, wherein the cells in the tissue comprise over 1,000,000,000 cells.

65. The system of any one of claims 58-64, wherein the nanoelectronic devices are tissue-like.

66. The method of any one of claims 58-65, wherein the nanoelectronic devices comprise a polymeric network.

67. The system of any one of claims 58-66, wherein the nanoelectronic devices comprise stretchable mesh.

68. The system of claim 67, wherein the stretchable mesh comprises an overall filling ratio of less than 100%, less than 50%, less than 20%, less than 15%, less than 10%, less than 5%, or less than 1%.

69. The system of claim 67, wherein the stretchable mesh comprises an overall filling ratio of less than 11%.

70. The system of any one of claims 58-69, wherein the nanoelectronic devices are embedded in a serpentine layout, a hexagonal layout, a triangular layout, or a straight layout.

71. The system of any one of claims 58-70, wherein the nanoelectronic devices comprise a mass of less than 50 μg, less than 40 μg, less than 30 μg, less than 20 μg, or less than less than 10 μg.

72. The system of any one of claims 58-70, wherein the nanoelectronic devices comprise a mass of less than 15 μg.

73. The system of any one of claims 58-72, wherein the nanoelectronic devices comprise a top encapsulation layer.

74. The system of claim 73, wherein the top encapsulation layer is an SU-8 encapsulation layer.

75. The system of any one of claims 58-74, wherein the nanoelectronic devices comprise an electrode layer.

76. The system of claim 75, wherein the electrode layer is a platinum electrode layer.

77. The system of claim 75 or 76, wherein the electrode layer comprises a coating.

78. The system of claim 77, wherein the coating is a poly(3,4-ethylenedioxythiophene) coating, a polyanaline coating, or a polypyrrole coating.

79. The system of any one of claims 58-78, wherein the nanoelectronic devices comprise a gold interconnecting layer.

80. The system of any one of claims 58-79, wherein the nanoelectronic devices comprise a bottom encapsulation layer.

81. The system of claim 80, wherein the bottom encapsulation layer is an SU-8 encapsulation layer.

82. The system of any one of claims 58-81, wherein the nanoelectronic devices comprise input and output lines.

83. The system of any one of claims 58-82, wherein the nanoelectronic devices comprise an electrical device, an optical device, a mechanical sensor, a stimulator, or an actuator.

84. The system of any one of claims 58-83, wherein the nanoelectronic devices are embedded within the cells in the tissue by transferring the nanoelectronic devices onto a two-dimensional sheet of cells and allowing the cells to aggregate, associate, proliferate, and migrate.

85. The system of claim 84, wherein the allowing the cells to aggregate, associate, proliferate, and migrate compresses the nanoelectronic devices and embeds them within the cells.

86. The system of any one of claims 58-85, wherein the cells are living.

87. The system of any one of claims 58-86, wherein the cells are in vivo.

88. The system of any one of claims 58-87, wherein the tissue is living.

89. The system of any one of claims 58-88, wherein the tissue is in vivo.

90. The system of any one of claims 58-89, wherein the tissue is three-dimensional.

91. The system of any one of claims 58-90, wherein the tissue is a tissue with electrical activity.

92. The system of any one of claims 58-91, wherein the tissue is brain tissue.

93. The system of any one of claims 58-91, wherein the tissue is heart tissue.

94. The system of any one of claims 58-91, wherein the tissue is pancreas tissue.

65. The system of any one of claims 58-91, wherein the tissue is nervous system tissue.

96. The system of any one of claims 58-91, wherein the tissue is muscle tissue.

97. The system of any one of claims 58-91, wherein the tissue is gastrointestinal tract tissue.

98. The system of any one of claims 58-91, wherein the tissue is developing tissue.

99. The system of any one of claims 58-91, wherein the tissue is diseased tissue.

100. The system of any one of claims 58-99, wherein the tissue is an organoid.

101. The system of any one of claims 58-100, wherein the tissue is derived from human induced pluripotent stem cells.

102. The system of any one of claims 58-101, wherein the cells are stem cells.

103. The system of any one of claims 58-102, wherein the cells are progenitor cells.

104. A method of preparing a tissue for continuous electrophysiological recording, the method comprising embedding one or more nanoelectronic devices in cells in the tissue to form a nanoelectronics-tissue hybrid, wherein each nanoelectronic device comprises at least one sensor with a unique electronic barcode.

105. The method of claim 104, wherein the electronic barcode is a fluorescence electronic barcode.

106. The method of claim 104 or claim 105, wherein over 1000, over 10,000, over 100,000, or over 1,000,000 nanoelectronic devices are embedded within the tissue.

107. The method of any one of claims 104-106, wherein the cells in the tissue comprise over 1,000,000 cells.

108. The method of any one of claims 104-107, wherein the cells in the tissue comprise over 1,000,000,000 cells.

109. The method of any one of claims 104-108, wherein the nanoelectronic devices are tissue-like.

110. The method of any one of claims 104-109, wherein the nanoelectronic devices comprise a polymeric network.

111. The method of any one of claims 104-110, wherein the nanoelectronic devices comprise stretchable mesh.

112. The method of claim 104-111, wherein the stretchable mesh comprises an overall filling ratio of less than 100%, less than 50%, less than 20%, less than 15%, less than 10%, less than 5%, or less than 1%.

113. The method of claim 104-112, wherein the stretchable mesh comprises an overall filling ratio of less than 11%.

114. The method of any one of claims 104-113, wherein the nanoelectronic devices are embedded in a serpentine layout, a hexagonal layout, a triangular layout, or a straight layout.

115. The method of any one of claims 104-114, wherein the nanoelectronic devices comprise a mass of less than 50 μg, less than 40 μg, less than 30 μg, less than 20 μg, or less than less than 10 μg.

116. The method of any one of claims 104-114, wherein the nanoelectronic devices comprise a mass of less than 15 μg.

117. The method of any one of claims 104-114, wherein the nanoelectronic devices comprise a top encapsulation layer.

118. The method of claim 117, wherein the top encapsulation layer is an SU-8 encapsulation layer.

119. The method of any one of claims 104-118, wherein the nanoelectronic devices comprise an electrode layer.

120. The method of claim 119, wherein the electrode layer is a platinum electrode layer.

121. The method of claim 119 or 120, wherein the electrode layer comprises a coating.

122. The method of claim 121, wherein the coating is a poly(3,4-ethylenedioxythiophene) coating, a polyanaline coating, or a polypyrrole coating.

123. The method of any one of claims 104-122, wherein the nanoelectronic devices comprise a gold interconnecting layer.

124. The method of any one of claims 104-123, wherein the nanoelectronic devices comprise a bottom encapsulation layer.

125. The method of claim 124, wherein the bottom encapsulation layer is an SU-8 encapsulation layer.

126. The method of any one of claims 104-125, wherein the nanoelectronic devices comprise input and output lines.

127. The method of any one of claims 104-126, wherein the nanoelectronic devices comprise an electrical device, an optical device, a mechanical sensor, a stimulator, or an actuator.

128. The method of any one of claims 104-127, wherein the step of embedding the nanoelectronic devices comprises transferring the nanoelectronic devices onto a two-dimensional sheet of cells and allowing the cells to aggregate, associate, proliferate, and migrate.

129. The method of claim 128, wherein allowing the cells to aggregate, associate, proliferate, and migrate compresses the nanoelectronic devices and embeds them within the cells.

130. The method of any one of claims 104-129, wherein the cells are living.

131. The method of any one of claims 104-130, wherein the cells are in vivo.

132. The method of any one of claims 104-131, wherein the tissue is living.

133. The method of any one of claims 104-132, wherein the tissue is in vivo.

134. The method of any one of claims 104-133, wherein the tissue is three-dimensional.

135. The method of any one of claims 104-134, wherein the tissue is a tissue with electrical activity.

136. The method of any one of claims 104-135, wherein the tissue is brain tissue.

137. The method of any one of claims 104-135, wherein the tissue is heart tissue.

138. The method of any one of claims 104-135, wherein the tissue is pancreatic tissue.

139. The method of any one of claims 104-135, wherein the tissue is nervous method tissue.

140. The method of any one of claims 104-135, wherein the tissue is muscle tissue.

141. The method of any one of claims 104-135, wherein the tissue is gastrointestinal tract tissue.

142. The method of any one of claims 104-135, wherein the tissue is developing tissue.

143. The method of any one of claims 104-135, wherein the tissue is a diseased tissue.

144. The method of any one of claims 104-143, wherein the tissue is an organoid.

145. The method of any one of claims 104-144, wherein the tissue is derived from human induced pluripotent stem cells.

146. The method of any one of claims 104-145, wherein the cells are stem cells.

147. The method of any one of claims 104-146, wherein the cells are progenitor cells.

148. A kit for correlating electrophysiological activity and gene expression in cells in a tissue comprising nanoelectronic devices within cells in a tissue, wherein each nanoelectronic device comprises at least one sensor with a unique electronic barcode.

149. The kit of claim 148, wherein the electronic barcode is a fluorescence electronic barcode.

150. A kit comprising the system of any one of claims 58-103.

151. A method for discovering a target for treating a disease, the method comprising correlating electrophysiological activity and gene expression in cells in a tissue by the steps of:
(a) embedding one or more nanoelectronic devices in a first tissue to form a nanoelectronics-tissue hybrid, wherein each nanoelectronic device comprises at least one sensor with a unique electronic barcode;
(b) performing continuous electrophysiological recording on the cells;
(c) fixing the nanoelectronics-tissue hybrid;
(d) performing in situ single-cell transcriptome sequencing on the cells;
(e) performing transcriptomic mapping on the cells;
(f) identifying the position of the electronic barcode within the nanoelectronics-tissue hybrid;
(g) performing cell segmentation to correlate the single-cell transcriptome sequencing data with the electrophysiological recording data; and
(h) repeating steps (a)-(g) on a second tissue, wherein the second tissue is engineered as a disease model, and comparing the single-cell transcriptome data and electrophysiological recording data from the first tissue and the second tissue.

152. A method of screening for a drug to treat a disease, the method comprising correlating electrophysiological activity and gene expression in cells in a tissue, wherein the tissue is engineered as a disease model, by the steps of:
(a) embedding one or more nanoelectronic devices in the tissue to form a nanoelectronics-tissue hybrid, wherein each nanoelectronic device comprises at least one sensor with a unique electronic barcode;
(b) performing continuous electrophysiological recording on the cells;
(c) fixing the nanoelectronics-tissue hybrid;
(d) performing in situ single-cell transcriptome sequencing on the cells;
(e) performing transcriptomic mapping on the cells;
(f) identifying the position of the electronic barcode within the nanoelectronics-tissue hybrid;
(g) performing cell segmentation to correlate the single-cell transcriptome sequencing data with the electrophysiological recording data; and
(h) repeating steps (a)-(g) in the presence of a drug and comparing the single-cell transcriptome sequencing data and the electrophysiological recording data with the data obtained in the absence of the drug.

153. The method of any one of claim 7, 151, or 152, wherein the step of continuous electrophysiological recording is performed for more than 1 day.

154. The method of any one of claim 7, 151, or 152, wherein the step of continuous electrophysiological recording is performed for more than 1 month.

155. The method of any one of claim 7, 151, or 152, wherein the step of continuous electrophysiological recording is performed for more than 1 year.

156. A nanoelectronic device comprising one or more sensors, wherein the one or more sensors each comprise a unique electronic barcode.

157. The nanoelectronic device of claim 156, wherein the electronic barcode is a fluorescence electronic barcode.

158. The nanoelectronic device of claim 156 or 157, wherein the electronic barcode comprises a unique binary code.
